(19) United States
(12) Patent Application Publication
(10) Pub. No.: US 2026/0234671 A1
(43) Pub. Date: Aug. 13, 2026

(54) Genomic Safe Harbors for Genetic Therapies in Human Stem Cells and Engineered Nanoparticles to Provide Targeted Genetic Therapies

(71) Applicant: Fred Hutchinson Cancer Center, Seattle, WA (US)

(72) Inventors: Jennifer E. Adair, Seattle, WA (US); Reza Shahbazi, Seattle, WA (US)

(21) Appl. No.: 19/358,321

(22) Filed: Oct. 14, 2025

Related U.S. Application Data
(63) Continuation of application No. 18/063,511, filed on Dec. 8, 2022, now Pat. No. 12,442,021.
Application No. 18/063,511 is a continuation of application No. 16/619,211, filed on Jun. 5, 2018, now Pat. No. 11,603,544.
(60) Provisional application No. 62/515,474, filed on Jun. 5, 2017.
(60) Provisional application No. 62/564,129, filed on Sep. 27, 2017.
(60) Provisional application No. 62/664,045, filed on Apr. 27, 2018.

(51) Int. Cl. C12N 15/90; A61K 40/10; A61K 40/40; A61K 47/69; C12N 5/078; C12N 9/22; C12N 15/113

(52) U.S. Cl. CPC C12N 15/907; A61K 40/10; A61K 40/40; A61K 47/6929; C12N 5/0634; C12N 9/22; C12N 15/113; A61K 2239/31; A61K 2239/38; C12N 2510/00

(57) ABSTRACT

Genomic safe harbors (GSH) for genetic therapies in human stem cells and engineered nanoparticles to provide targeted genetic therapies are described. The GSH and/or associated nanoparticles can be used to safely and efficiently treat a variety of genetic, infectious, and malignant diseases.

### CROSS-REFERENCE TO RELATED APPLICATIONS

This application a Continuation of U.S. patent application Ser. No. 18/063,511, filed Dec. 8, 2022, which is a Continuation of U.S. application Ser. No. 16/619,211 filed on Dec. 4, 2019, now U.S. Pat. No. 11,603,544, which is a U.S. National Phase Application based on International Patent Application No. PCT/US2018/036154 filed on Jun. 5, 2018, which claims priority to U.S. Provisional Patent Application No. 62/515,474 filed on Jun. 5, 2017, U.S. Provisional Patent Application No. 62/564,129 filed on Sep. 27, 2017, and U.S. Provisional Patent Application No. 62/664,045 filed on Apr. 27, 2018, each of which is incorporated by reference in its entirety as if fully set forth herein.

### REFERENCE TO SEQUENCE LISTING

This application contains a Sequence Listing that has been submitted electronically as an XML file named “0791-0462003_SL_ST26.XML.” The XML file, created on Jan. 29, 2026 and is 310,801 bytes in size. The material in the XML file is hereby incorporated by reference in its entirety.

### FIELD OF THE DISCLOSURE

The current disclosure provides genomic safe harbors (GSH) for genetic therapies in human stem cells and engineered nanoparticles to provide targeted genetic therapies. The disclosed GSH and/or associated nanoparticles can be used to safely and efficiently treat a variety of genetic, infectious, and malignant diseases.

### BACKGROUND OF THE DISCLOSURE

Patient-specific gene therapy has great potential to treat genetic, infectious, and malignant diseases. For example, retrovirus-mediated gene addition into hematopoietic stem cells (HSC) and hematopoietic stem cells and progenitor cells (HSPC) has demonstrated curative outcomes for several genetic diseases over the last 10 years including inherited immunodeficiencies (e.g., X-linked and adenosine deaminase deficient severe combined immunodeficiency (SCID)), hemoglobinopathies, Wiskott-Aldrich syndrome and metachromatic leukodystrophy. Additionally, this treatment approach has also improved outcomes for poor prognosis diagnoses such as glioblastoma. The use of gene-corrected autologous, or “self”, HSPC eliminates the risk of graft-host immune responses, negating the need for immunosuppressive drugs. However, effective implementation of HSPC gene therapy faces several major challenges. The current state-of-the-art includes the removal of cells from the patient via bone marrow aspirate or mobilized peripheral blood, sorting this bulk population for autologous HSPC by immunoselection of cells expressing the surface marker CD34, then culturing these cells in the presence of cytokines and the specified therapeutic retrovirus vector before harvesting. Re-administering cells to the patient may require cytoreductive conditioning to permit engraftment of the gene corrected cells. Currently, only centers with Good Manufacturing Practices (GMP) compliant facilities and the infrastructure to support them are capable of administering gene modified cell products. While a simplified manufacturing platform to automate this process in a small, mobile footprint has been developed, severely limited quantities of available therapeutic vectors have continued to create a significant bottleneck to widespread use of the technology.

In addition to the challenge of manufacturing sufficient therapeutic vector quantities, there remains a known risk of genotoxicity and other limitations associated with the use of viral vectors for gene transfer. For example, risks of genotoxicity are evidenced by the development of malignancy due to insertional mutagenesis in patients treated with HSPC gene therapy. This adverse side effect stems from the semi-random nature of retroviral-mediated transgene delivery into the host cell genome. Dysregulation of nearby genes by the inserted transgene sequence has been the molecular basis for clonal expansion and malignant transformation observed in some gene therapy patients, but reciprocal interactions between the inserted transgene and the surrounding genomic context can also cause transgene attenuation or silencing, diminishing therapeutic effects. Other limitations associated with the use of particular viral vectors include induction of immune responses, a decreased efficacy over time in dividing cells (e.g., adeno-associated vectors), an inability to adequately target selected cell types in vivo (e.g., retroviral vectors), and, as indicated, an inability to control insertion site and number of insertions (e.g., lentiviral vectors).

The last 5 years have seen an explosion in gene editing as a safer alternative to retrovirus-mediated gene transfer, made possible by the development of engineered guide RNA and nucleases which target specific DNA sequences and predictably generate DNA double strand breaks (DSB) at the targeted sequence. To date, these programmable complexes have been most effective at providing promising therapies when removal or silencing of a problematic gene (i.e., generating a loss-of-function mutation) is needed. This is because DSBs are most commonly repaired by error-prone non-homologous end joining (NHEJ) which results in oligonucleotide insertions and deletions (indels) at the DSB site.

For gene addition or correction of a specific mutation, less common homology-directed repair (HDR) of the DSB is required. In this situation, a more complex payload including the engineered guide RNA and nuclease as well as a homology-directed repair template must be co-delivered. Proof-of-concept for this approach has been demonstrated in HSPC but also required either tandem electroporation of some gene editing components followed by transduction with non-integrating viral vectors, particularly recombinant adeno-associated viral (rAAV) vectors to deliver DNA templates, or simultaneous electroporation of defined concentrations of engineered nuclease components with chemically modified, single-stranded oligonucleotide template at specified cell concentrations. Moreover, each engineered guide RNA, nuclease and homology-directed repair template had to be uniquely engineered for each specified genetic target, requiring separate evaluation of delivery, activity and specificity in cell lines and HSPC.

Thus, while there have been many exciting breakthroughs in the ability to perform genetic therapies at specific sites within the genome, the continued lack of a safe and potent delivery vehicle has hindered the clinical translation of gene editing systems, in particular, with HSPCs.

The concept of a genomic safe harbor (GSH) for genetic modification was first introduced in 2011 by Papapetrou and colleagues (Nature Biotechnology. 2011; 29(1):73-8). The major criteria proposed to define a GSH site are (1) the ability to accommodate new genetic material with, (2) predictable function, and (3) without potentially harmful alterations in host cell genomic activity. The benefit of identifying such a locus would greatly simplify development efforts for targeted gene addition approaches. Several loci have been evaluated in the human genome, but to date no bona fide validated GSH sites that meet the above criteria have been identified. Papapetrou et al., Mol. Ther. 2016; 24(4): 678-84.

### SUMMARY OF THE DISCLOSURE

The current disclosure provides significant advances in the ability to perform genetic therapies for a variety of genetic, infectious, and malignant diseases by providing the identification of genomic safe harbors (GSH) within human hematopoietic stem cells (HSC) and hematopoietic stem cells and progenitor cells (HSPC). In particular embodiments, the GSH additionally qualify as the more rigorously defined universal HSC safe harbor loci, as described in additional detail herein.

The current disclosure also provides nanoparticles specifically engineered to deliver all components required for genetic editing, for example, at the GSH sites. The nanoparticles can be used for therapies where a loss-of-function mutation is needed, but importantly, can also provide all components needed for gene addition or correction of a specific mutation. The described approaches are safe (i.e., no off-target toxicity), reliable (targeted GSH cell chromatin is accessible and amenable to therapeutic cassette addition), scalable, easy to manufacture, synthetic, plug-and-play (i.e., the same basic platform can be used to deliver different therapeutic nucleic acids), and compatible with easy in vivo administration (through, for example, a syringe).

Particular embodiments include a nanoparticle with components to provide a targeted loss-of-function mutation. These embodiments include a targeting element (e.g., guide RNA) and a cutting element (e.g. a nuclease) associated with the surface of the nanoparticle. In particular embodiments, the targeting element is conjugated to the surface of the nanoparticle through a thiol linker. In particular embodiments, the targeting element and/or the cutting element are conjugated to the surface of the nanoparticle through a thiol linker. In particular embodiments, the targeting element is conjugated to the surface of the nanoparticle through a thiol linker and the cutting element is linked to the targeting element to form a ribonucleoprotein (RNP) complex. The targeting element targets the cutting element to a specific site for cutting and NHEJ repair.

Particular embodiments include a nanoparticle with components to provide a targeted gain-of-function mutation (e.g., gene addition or correction). In particular embodiments, these embodiments include a metal nanoparticle (e.g., a gold nanoparticle) associated with a targeting element, a cutting element, a homology-directed repair template, and a therapeutic DNA sequence. The targeting element targets the cutting element to a specific site for cutting, the homology-directed repair template provides for HDR repair, wherein following HDR repair the therapeutic DNA sequence has been inserted within the target site. Together, homology-directed repair templates and therapeutic DNA sequences can be referred to herein as donor templates. In particular embodiments, the targeting element is conjugated to the surface of the nanoparticle through a thiol linker. In particular embodiments, the targeting element and/or the cutting element are conjugated to the surface of the nanoparticle through a thiol linker. In particular embodiments, the targeting element is conjugated to the surface of the nanoparticle through a thiol linker and the cutting element is linked to the targeting element to form a ribonucleoprotein (RNP) complex. In these embodiments, the RNP complex is closer to the surface of the nanoparticle than donor template material. This configuration is beneficial when, for example, the targeting element and/or the cutting element are of bacterial origin. This is because many individuals who may receive nanoparticles described herein may have pre-existing immunity against bacterially-derived components such as bacterially-derived gene-editing components. Including bacterially-derived gene-editing components on an inner layer of the fully formulated nanoparticle allows non-bacterially-derived components (e.g., donor templates) to shield bacterially-derived components (e.g. targeting elements and/or cutting elements) from the patient's immune system. This protects the bacterially-derived components from attack and also avoids or reduces unwanted inflammatory responses against the nanoparticles following administration. In addition, this may allow for repeated administration of the nanoparticles in vivo without inactivation by the host immune response.

Particular embodiments utilize CRISPR gene editing. In particular embodiments, CRISPR gene editing can occur with CRISPR guide RNA (crRNA) and/or a CRISPR nuclease (e.g., Cpf1 (also referred to as Cas12a) or Cas9).

Particular embodiments adopt features that increase the efficiency and/or accuracy of HDR. For example, Cpf1 has a short single crRNA and cuts target DNA in staggered form with 5′ 2-4 nucleotide (nt) overhangs called sticky ends. Sticky ends are favorable for HDR, Kim et al. (2016) Nat Biotechnol. 34(8): 863-8. Moreover, donor templates should be released from the nanoparticles before the genome cut by the RNP occurs to promote HDR. Accordingly, in particular embodiments disclosed herein donor templates are found farther from the surface of the nanoparticle than targeting elements and cutting elements. The current disclosure also unexpectedly found that delivery of gene-editing components on a gold nanoparticle increases the efficiency and/or accuracy of HDR. Accordingly, particular embodiments deliver gene-editing components utilizing gold nanoparticles.

In particular embodiments, targeting molecules can be used to target the nanoparticle to a specific cell so that activity of the gene editing system can be spatially or temporally controlled. For example, the activity and destination of the gene editing system may be controlled by a targeting molecule that selectively delivers the nanoparticle to targeted cells. In particular embodiments, the targeting molecule can include an antibody binding domain that binds CD34. In particular embodiments, pairs of targeting molecule can be used, for example, an antibody binding domain that binds CD34 and an antibody binding domain that binds CD90.

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings.

FIG. 1. Schematic showing the design and sequence of clustered regularly interspaced short palindromic repeat (CRISPR) RNA (crRNA) (SEQ ID NO: 244) and homology templates (HT) (SEQ ID NOs: 247, 248) for a genomic safe harbor (GSH) location (SEQ ID NOs: 242, 243, 245, 246) to allow targeted genome editing within human hematopoietic stem cells (HSC) and hematopoietic stem cells and progenitor cells (HSPC).

FIG. 2. A trace of a Sanger sequencing result showing a mutation in one target GSH site in K562 cells (SEQ ID NOs: 249 and 250).

FIG. 3. Schematic showing the design and sequence of crRNA (SEQ ID NO: 253) and homology-directed repair template (also referred to herein as a homology template (HT or HDT)) (SEQ ID NOs: 256, 257) for a second GSH location (SEQ ID NOs: 251, 252, 254, 255) within human HSC and HSPC.

FIG. 4. Two GSH sites (SEQ ID NOs: 258, 259) within the human CCR5 gene within chromosome 3p21 along with crRNA (SEQ ID NO: 260) and HT (SEQ ID NOs: 261, 262) for a non-target and target strand. The purpose of choosing CCR5 was to validate the nanoformulation. A well-known naturally-occurring mutation in the CCR5 gene renders a person's blood cells resistant to HIV infection. For purposes of the present disclosure, this site provided a means to compare Cas9 and Cpf1 nucleases directly because there is a DNA sequence near the site of this naturally-occurring mutation that contains an identical cut site for both Cpf1 and Cas9. The PAM mutation in the homology template for the target strand is italicized, bolded, and underlined.

FIG. 5. Schematic of early production scheme for gold nanoparticles (AuNPs), a scalable, synthetic delivery scaffold with established in vivo compatibility.

FIG. 6. AuNP size determines destination tissue/elimination pathway in humans.

FIGS. 7A-7D. Schematics depicting an exemplary AuNP associated with all gene editing components required for gene addition and/or correction. (FIG. 7A) Depiction of an exemplary AuNP configured with all components for gene addition within a GSH. Depicted components include crRNA, a Cpf1 nuclease, and ssDNA to provide a therapeutic nucleic acid sequence (e.g. a gene or corrected portion thereof). The embodiment depicted in FIG. 7A additionally includes a targeting molecule (e.g., an antibody binding domain or an aptamer). (FIG. 7B) Schematic representation of a synthesis process for creating and loading AuNP with exemplary gene editing components. (FIG. 7C) Schematic of a formulated “layered” AuNP which can be used to deliver large oligonucleotides, such as donor templates including homology-directed repair templates, therapeutic DNA sequences, and other potential elements. Donor templates are located farther from the AuNP surface than the depicted ribonucleoprotein complex (RNP) (FIG. 7D) Successful generation of 50 nm AuNP carrying all required gene editing components for insertion of a therapeutic DNA sequence. Transmission electron microscope images of AuNPs and AuNP/CRISPR nanoformulations. The depicted embodiment includes an AuNP associated with crRNA, Cpf1, ssDNA, thiol linkers, polyethylene glycol (PEG) spacers, and PEI2K. More particularly, thiol linkers are attached directly to the surface of the AuNP and crRNA. Use of such a linker allows conjugation of more crRNA to the surface of the AuNP, in part because the spaces reduces repulsion between the negative charge of RNA-based molecules and the negative charge of the AuNP surface. Cpf1 is linked to the crRNA to form an RNP. To improve the efficiency of HDR, ssDNA should be released from the AuNP before the genome cut occurs. Accordingly, ssDNA is found farther from the surface of the AuNP than the RNP.

FIG. 8. Fully-loaded AuNPs are monodisperse and display good zeta potential.

FIGS. 9A-9D. Graphs and digital images showing the characteristic properties of synthesized AuNPs and optimal loading concentrations. (FIG. 9A) Localized surface plasmon resonance (LSPR) peaks of synthesized AuNPs. (FIG. 9B) LSPR peaks of the AuNP and AuNP/CRISPR nanoformulations. (FIG. 9C) Gel electrophoresis showing optimal AuNP/ssDNA w/w loading ratio. (FIG. 9D) Loading concentration of AuNP/CRISPR nanoformulations.

FIGS. 10A-10C. Optimal loading concentrations. (FIG. 10A) AuNP/crRNA 50 nm (Ratio 6); AuNP/crRNA 15 nm (Ratio 1); and AuNP/crRNA/Cpf1/PEI/DNA 15 nm (Ratio 0.5); (FIG. 10B) Optimal AuNP 50 nm/DNA w/w ratio; (FIG. 10C) Smaller AuNPs triple the available surface area with the same starting reagent amounts. By decreasing the size, surface area and conjugation ratio of the NPs increase.

FIGS. 11A, 11B. (FIG. 11A) HSPC take up fully-loaded AuNPs in vitro. Time lapse imaging on spinning disc confocal microscope. Human mobilized CD34+ cells→Culture O/N→Add AuNPs (4 hours)→Analyze. (FIG. 11B) Confocal microscope imaging showing the uptake of CRISPR editing components into CD34+ cells.

FIGS. 12A-12D. Graphs showing the gene cutting efficiency in K562 cells and CD34+ cells. (FIGS. 12A, 12B) Tracking Indels by Decomposition (TIDE) assay results showing percent cutting efficiency in K562 cells and CD34+ cells. (FIG. 12C) Percent viability after delivery with AuNPs and electroporation method. (FIG. 12D) Administration dose of CRISPR components.

FIG. 13. Up to 10% gene editing and HDR was observed in vitro in primary CD34+ cells obtained from a G-CSF mobilized healthy adult donor. CD34+ cells were thawed using a rapid-thaw method and cultured overnight in Iscove's Modified Dulbecco's Medium (IMDM) containing 10% FBS and 1% Pen/Strep. The following morning, AuNPs were seeded and assembled as follows: seed; add crRNA with a PEG spacer to prevent electrostatic repulsions; add Cpf1 protein and allow RNPs to form; coat with 2K branched PEI and ssODN. In this example, there were no chemical modifications of crRNA other than terminal thiol additions to promote covalent bonding with the AuNP surface for attachment. SsODN was used as the homology-directed repair template (HDT), here a 8 bp insert using a NotI site flanked by 40 nt of homology (symmetric) to CCR5 target locus. Formulated AuNPs were added to cells and incubated for 48 hours with gentle plate mixing. After 48 hours, cells were harvested, washed, and gDNA was isolated for PCR amplification and analysis.

FIG. 14. TIDE assay results showing indels after editing with AuNP/CRISPR nanoformulations in CD34+ cells.

FIGS. 15A-15D. (FIG. 15A) 10 μg/mL AuNP is an optimal concentration for HDR in CD34+ cells where maximum gene editing is achieved without loss in viability. The same protocol was used as described in relation to FIG. 13, except that CD34+ cells were initially obtained from a different human donor. (FIGS. 15B, 15C) Cell viability analysis by Live-Dead assay; (FIG. 15D) representative cell viability results in bar graph format.

FIG. 16. The goal of the study leading to the data presented in FIG. 16 was to compare Cpf1 and Cas9 delivered by AuNP or electroporation, respectively. The same procedures were used as described in relation to FIG. 13, except that CD34+ cells were initially obtained from a different human donor. All formulation conditions were the same such that the only variables are Cpf1 vs. Cas9; AuNP vs. Electroporation; homology-directed repair template (HDT; ssODN) present vs. absent. Electroporation conditions: 1 mm electroporation cuvets; 125 mV; 5 ms. Editing analysis by TIDE. A CCR5 non-target sequence of CCR5 locus is depicted (SEQ ID NO: 263).

FIGS. 17A-17C. Serum-free, HSPC-supportive media conditions improve (17A) editing, (17B) HDR, and (17C) cell viability.

FIGS. 18A, 18B. (FIG. 18A) There is no significant impact on HSPC fitness in vitro by any nuclease or delivery method as determined by a first colony-forming assay in methylcellulose (H4434). CD34+ cells were seeded at 200 cells per 35 mm plate with a total incubation time of 14 days. (FIG. 18B) Improved HSPC secondary plating fitness in vitro with AuNP/Cpf1. Colony plates from (FIG. 18A) were harvested and 10% of the resulting cell suspension was re-plated into a second colony-forming assay in Methylcellulose (H4434). This is a simple test to determine whether long-term colony-initiating cells were impacted by the experimental conditions. The total incubation time was 14 days. The only significant difference observed was in the number of colonies for the AuNP+Cpf1/crRNA treated cells, which displayed more long-term colonies-forming cells. No significant differences were observed in the type of colonies formed in each experimental group.

FIG. 19. AuNP-treated CD34+ cells engraft in vivo. Experimental notes and methodology. The same procedures were used as described in relation to FIG. 13, except that CD34+ cells were initially obtained from a different human donor. After 48 hours, cells were harvested, washed, and injected into sublethally irradiated adult (8-12 week) NSG mice. Cell reserves were used to assess plate colony assays and to isolate gDNA for PCR amplification and analysis.

FIGS. 20A-20C. In vitro analysis of cells transplanted into NSG mice. (FIG. 20A) 10% HDR was observed by TIDE without significant indels at the target locus in human CD34+ cells at the time of transplant. (FIG. 20B) Both T7EI and NotI restriction digest were only observed in cells that received fully-loaded AuNP nanoformulations. (FIG. 20C) Interestingly, increased colony-forming capacity for this donor was noted only when cells were treated with AuNPs. No significant differences were observed in the types of colonies formed across each condition.

FIGS. 21A-21J. In vivo analysis of cells transplanted into NSG mice. AuNP-treated hCD34+ cells engraft better than mock-treated cells. (FIGS. 21A, 21B) Mice transplanted with AuNP-treated cells displayed higher levels of human CD45+ cell engraftment than mice transplanted with mock cells. (FIGS. 21C, 21D) No significant differences in the frequency of human CD20+ cells were observed across groups. (FIGS. 21E, 21F) No significant differences in the frequency of human CD14+ cells were observed across groups. (FIGS. 21G, 21H) Significant differences in the frequency of human CD3+ cells were observed at 14 weeks and in the mice receiving fully-loaded AuNPs who displayed the lowest overall human engraftment. This result may be an artifact of low engraftment levels.

FIG. 22. Early post-transplant analysis suggests gene edited cell engraftment. Peripheral blood was collected for gDNA analysis at 6 weeks after transplant (arrow in FIG. 211). Across all mice treated with fully-loaded AuNPs, 7/10 displayed detectable editing ranging from 0.5-6% by TIDE. In one mouse (5% total editing), 1.7% HDR was observed by TIDE analysis.

FIG. 23. Sequences supporting the disclosure.

### DETAILED DESCRIPTION

Patient-specific gene therapy has great potential to treat genetic, infectious, and malignant diseases. For example, retrovirus-mediated gene addition into hematopoietic stem cells (HSC) and hematopoietic stem cells and progenitor cells (HSPC) has demonstrated curative outcomes for several genetic diseases over the last 10 years including inherited immunodeficiencies (e.g., X-linked and adenosine deaminase deficient severe combined immunodeficiency (SCID)), hemoglobinopathies, Wiskott-Aldrich syndrome and metachromatic leukodystrophy. Additionally, this treatment approach has also improved outcomes for poor prognosis diagnoses such as glioblastoma. The use of gene-corrected autologous, or “self”, HSPC eliminates the risk of graft-host immune responses, negating the need for immunosuppressive drugs. However, effective implementation of HSPC gene therapy faces several major challenges. The current state-of-the-art includes the removal of cells from the patient via bone marrow aspirate or mobilized peripheral blood, sorting this bulk population for autologous HSPC by immunoselection of cells expressing the surface marker CD34, then culturing these cells in the presence of cytokines and the specified therapeutic retrovirus vector before harvesting. Re-administering cells to the patient may require cytoreductive conditioning to permit engraftment of the gene corrected cells. Currently, only centers with Good Manufacturing Practices (GMP) compliant facilities and the infrastructure to support them are capable of administering gene modified cell products. While a simplified manufacturing platform to automate this process in a small, mobile footprint has been developed, severely limited quantities of available therapeutic vectors have continued to create a significant bottleneck to widespread use of the technology.

In addition to the challenge of manufacturing sufficient therapeutic vector quantities, there remains a known risk of genotoxicity associated with the use of retroviral vectors for gene transfer evidenced by the development of malignancy due to insertional mutagenesis in patients treated with HSPC gene therapy. This adverse side effect stems from the semi-random nature of retroviral-mediated transgene delivery into the host cell genome. Dysregulation of nearby genes by the inserted transgene sequence has been the molecular basis for clonal expansion and malignant transformation observed in some gene therapy patients, but reciprocal interactions between the inserted transgene and the surrounding genomic context can also cause transgene attenuation or silencing, diminishing therapeutic effects.

The last 5 years have seen an explosion in gene editing as a safer alternative to retrovirus-mediated gene transfer, made possible by the development of engineered guide RNA associated with nucleases which target specific DNA sequences and predictably generate DNA double strand breaks (DSB) at the targeted sequence. To date, these programmable complexes have been most effective at providing promising therapies when removal or silencing of a problematic gene (i.e., generating a loss-of-function mutation) is needed. This is because DSBs are most commonly repaired by error-prone non-homologous end joining (NHEJ) which results in oligonucleotide insertions and deletions (indels) at the DSB site.

For gene addition or correction of a specific mutation, less common homology-directed repair (HDR) of the DSB is required. In this situation, a more complex payload including the engineered guide RNA and nuclease, and a homology-directed repair template with homology to the target DSB locus must be co-delivered. Proof-of-concept for this approach has been demonstrated in HSPC but also required either tandem electroporation of genome editing components followed by transduction with non-integrating viral vectors, particularly recombinant adeno-associated viral (rAAV) vectors to deliver DNA templates, or simultaneous electroporation of defined concentrations of engineered nuclease components with chemically modified, single-stranded oligonucleotide template at specified cell concentrations. Moreover, each guide RNA, nuclease and homology-directed repair template had to be uniquely engineered for each specified genetic target, requiring separate evaluation of delivery, activity and specificity in cell lines and HSPC.

Thus, while there have been many exciting breakthroughs in the ability to perform genetic therapies at specific sites within the genome, the continued lack of a safe and potent delivery vehicle has hindered the clinical translation of gene editing systems, in particular, with HSPCs.

The concept of a genomic safe harbor (GSH) for genetic modification was first introduced in 2011 by Papapetrou and colleagues (Nature Biotechnology. 2011; 29(1):73-8). The major criteria proposed to define a GSH site are (1) the ability to accommodate new genetic material with, (2) predictable function, and (3) without potentially harmful alterations in host cell genomic activity. The benefit of identifying such a locus would greatly simplify development efforts for targeted gene addition approaches. Several loci have been evaluated in the human genome, but to date no bona fide validated GSH sites that meet the above criteria have been identified. Papapetrou et al., Mol. Ther. 2016; 24(4): 678-84.

The current disclosure provides significant advances in the ability to perform genetic therapies for a variety of genetic, infectious, and malignant diseases by providing the identification of genomic safe harbors (GSH) within human hematopoietic stem cells (HSC) and hematopoietic stem cells and progenitor cells (HSPC). Some of the identified GSH additionally qualify as the more rigorously defined universal HSC safe harbor loci, as described in additional detail herein.

The current disclosure also provides nanoparticles specifically engineered to deliver all components required for genetic editing, for example, at the GSH sites. The nanoparticles can be used for therapies where a loss-of-function mutation is needed, but importantly, can also provide all components needed for gene addition or correction of a specific mutation. The described approaches are safe (i.e., no off-target toxicity), reliable (targeted GSH cell chromatin is accessible and amenable to therapeutic additions), scalable, easy to manufacture, synthetic, plug-and-play (i.e., the same basic platform can be used to deliver different therapeutic nucleic acids), and compatible with easy in vivo administration (through, for example, a syringe).

Particular embodiments include a nanoparticle with components to provide a targeted loss-of-function mutation. These embodiments include a targeting element (e.g., guide RNA) and a cutting element (e.g. a nuclease) associated with the surface of the nanoparticle. In particular embodiments, the targeting element is conjugated to the surface of the nanoparticle through a thiol linker. In particular embodiments, the targeting element and/or the cutting element are conjugated to the surface of the nanoparticle through a thiol linker. In particular embodiments, the targeting element is conjugated to the surface of the nanoparticle through a thiol linker and the cutting element is linked to the targeting element to form a ribonucleoprotein (RNP) complex. The targeting element targets the cutting element to a specific site for cutting and NHEJ repair.

Particular embodiments include a nanoparticle with components to provide a targeted gain-of-function mutation (e.g., gene addition or correction). These embodiments include a targeting element, a cutting element, a homology-directed repair template, and a therapeutic DNA sequence associated with the surface of the nanoparticle. The targeting element targets the cutting element to a specific site for cutting, the homology-directed repair template provides for HDR repair, wherein following HDR repair the therapeutic DNA sequence has been inserted within the target site. Together, homology-directed repair templates and therapeutic DNA sequences can be referred to herein as donor templates. In particular embodiments, the targeting element is conjugated to the surface of the nanoparticle through a thiol linker. In particular embodiments, the targeting element and/or the cutting element are conjugated to the surface of the nanoparticle through a thiol linker. In particular embodiments, the targeting element is conjugated to the surface of the nanoparticle through a thiol linker and the cutting element is linked to the targeting element to form a ribonucleoprotein (RNP) complex. In these embodiments, the RNP complex is closer to the surface of the nanoparticle than donor template material. This configuration is beneficial when, for example, the targeting element and/or the cutting element are of bacterial origin. This is because many individuals who may receive nanoparticles described herein may have pre-existing immunity against bacterially-derived components, such as bacterially-derived gene-editing components. Including bacterially-derived gene-editing components on an inner layer of the fully formulated nanoparticle allows non-bacterially-derived components (e.g., donor templates) to shield bacterially-derived components (e.g. targeting elements and/or cutting elements) from the patient's immune system. This protects the bacterially-derived components from attack and also avoids or reduces unwanted inflammatory responses against the nanoparticles following administration. In addition, this may allow for repeated administration of the nanoparticles in vivo without inactivation by the host immune response.

Particular embodiments utilize CRISPR gene editing. In particular embodiments, CRISPR gene editing can occur with CRISPR guide RNA (crRNA) and/or a CRISPR nuclease (e.g., Cpf1 or Cas9).

Particular embodiments adopt features that increase the efficiency and/or accuracy of HDR. For example, Cpf1 has a short single crRNA and cuts target DNA in staggered form with 5′ 2-4 nucleotide (nt) overhangs called sticky ends. Sticky ends are favorable for HDR, Kim et al. (2016) Nat Biotechnol. 34(8): 863-8. Moreover, donor templates should be released from the nanoparticles before the genome cut by the RNP occurs to promote HDR. Accordingly, in particular embodiments disclosed herein donor templates are found farther from the surface of the nanoparticle than targeting elements and cutting elements. The current disclosure also unexpectedly found that delivery of gene-editing components on a gold nanoparticle increases the efficiency and/or accuracy of HDR. Accordingly, particular embodiments deliver gene-editing components utilizing gold nanoparticles.

In particular embodiments, targeting molecules can be used to target the nanoparticle to a specific cell so that activity of the gene editing system can be spatially or temporally controlled. For example, the activity and destination of the gene editing system may be controlled by a targeting molecule that binds a cell surface marker, such as CD34 or CD90.

In embodiments utilizing gene-editing components of bacterial origin, the current disclosure also takes into account that many individuals who may receive nanoparticles described herein may have pre-existing immunity against such components. To address this potential pre-existing immunity, gene-editing components of bacterial origin may be directly conjugated to the surface of nanoparticles followed by addition of donor templates. In this configuration, donor templates can shield the gene-editing components from immune attack and avoid or reduce unwanted inflammatory responses against the nanoparticles following administration.

The following aspects of the disclosure are now described with additional detail and options to support the teachings of the disclosure as follows: (I) Genomic Safe Harbors (GSH) and Universal HSC Safe Harbor Loci in Human HSC and HSPC; (II) Gene Editing Systems and Components to Target and Modify GSH Sites; (Ill) Nanoparticles; (IV) Conjugation of Active Components to Nanoparticles; (V) Gene Editing Efficiency; (VI) Nanoparticle Compositions and Cell Formulations; (VII) Exemplary Methods of Use; and (VIII) Reference Levels Derived from Control Populations; (IX) Kits; and (X) Exemplary Embodiments.

(I) Genomic Safe Harbors (GSH) and Universal HSC Safe Harbor Loci in Human HSC and HSPC. As indicated, one drawback with existing gene therapies is that the insertion site of retroviral vectors cannot be adequately controlled. Gene editing systems allow control over the target sites of genetic therapies, however, before the current disclosure, no bona fide validated GSH sites had been identified in the human genome (Papapetrou et al., Mol. Ther. 2016; 24(4): 678-84), as the concept had been proposed by Papapetrou and colleagues in Nature Biotechnology. 2011; 29(1):73-8) (i.e., (1) the ability to accommodate new genetic material with, (2) predictable function, and (3) without potentially harmful alterations in host cell genomic activity).

One of the challenges of the incorporation of genetic material in cells is determining where within the chromosomes the genetic material can be safely incorporated. The present disclosure solves this problem by providing chromatin-accessible regions in the CD34⁺ cell and CD34 subtype (CD45RA⁻ and CD90⁺) in human and non-human primate cells (see, e.g., WO 2017/218948 and Radtke et al., Sci. Transl. Med. 2017; 9 (414) which have high editing efficiency and low probability of disrupting cellular potential. In particular embodiments, the sites qualify as universal HSC safe harbor loci. In particular embodiments, to meet the criteria of a universal HSC safe harbor loci, chromatin sites must be >150 kb away from a known oncogene, >30 kb away from a known transcription start site; and have no overlap with coding mRNA. In particular embodiments, to meet the criteria of a universal HSC safe harbor loci, chromatin sites must be >200 kb away from a known oncogene, >40 kb away from a known transcription start site; and have no overlap with coding mRNA. In particular embodiments, to meet the criteria of a universal HSC safe harbor loci, chromatin sites must be >300 kb away from a known oncogene, >50 kb away from a known transcription start site; and have no overlap with coding mRNA. In particular embodiments, a universal HSC safe harbor loci meets the preceding criteria (>150 kb, >200 kb or >300 kb away from a known transcription start site; and have no overlap with coding mRNA >40 kb, or >50 kb away from a known transcription start site with no overlap with coding mRNA) and additionally is 100% homologous between the non-human primate and the human genome to permit rapid clinical translation of these gene edited populations. In particular embodiments, a universal HSC safe harbor loci meets the preceding criteria and demonstrates a 1:1 ratio of forward:reverse orientations of LV integration further demonstrating the loci does not impact surrounding genetic material.

The process to identify GSH within the human genome began by evaluating the biological outcome of long-term engraftment of lentivirus (LV) gene modified, autologous CD34⁺ cells in the pigtailed macaque (M. nemestrina), an established non-human primate model used for HSC and HSPC gene therapy preclinical evaluations. A high-throughput analysis of sites of LV integration was used to identify candidate GSH loci. LVs can transduce non-dividing cells, and integrate preferentially into active transcription units in the host cell genome. The locus of integration is determined at the time of gene transfer and is inherited by each daughter cell. 150,000 LV integration sites identified in blood cells collected from twelve animals over a period of 2-7 years after transplant were parsed into 1,077 25 kb genomic windows displaying significantly enriched frequencies of integration relative to the rest of the genome (Table 1).

TABLE 1
Identification of candidate GSH loci from LV integration sites identified
in vivo in nonhuman primate recipients of autologous HSPC therapy

| | Number |
|---|---|
| Parsing Step Performed (Top to Bottom) | of Events |
| Total IS identified in 12 monkeys | 148,283 |
| Total number of 25 kb genomic windows containing IS | >95,000 |
| 25 kb genomic windows significantly enriched for IS | 1,077 |
| 25 kb genomic windows with 1:1 ratio of forward:reverse IS | 664 |
| orientation | |
| 25 kb genomic windows containing IS identified in 3 or more | 662 |
| biological replicates | |
| 25 kb windows with ≥90% homology to human genome | 171 |
| 25 kb windows ≥300 kb away from known oncogenes | 122 |
| 25 kb windows ≥50 kb away from TSS | 24 |
| 25 kb windows associated with constitutive transcriptional | 2 |
| activity in whole blood | |

IS: integration sites
TSS: transcription start site

A benign accessible locus would be expected to display a 1:1 ratio of forward:reverse orientations of LV integration. The list was thus further parsed into 664 genomic windows with equivalent forward and reverse orientation of integration events. Of these, 662 windows contained integration events which were represented by 3 or more biological replicates (>3 of 12 monkeys analyzed).

The windows were filtered based on homology to the human genome (hg38) and a total of 171 windows were identified with 90% homology. To increase safety, these windows were cross-referenced against the COSMIC cancer gene database. Windows were only retained if they were >300 kb away from a known oncogene. This filter resulted in 122 windows. Any windows within 50 kb of a transcription start site were removed, which resulted in 24 windows, all of which were preferentially located in intronic sequences. Two genomic regions were highly enriched in these 24 windows: chromosome 11q13.2 and chromosome 16p12.1.

Both of these gene-rich loci are constitutively expressed in blood cells, indicating that (1) expression of transgenes is not expected to transactivate nearby genes which should be silenced in blood cells, and (2) inserted transgene sequences will not be attenuated or silenced during hematopoietic differentiation [University of California at Santa Cruz (UCSC) Genome Browser and ENCODE]. These two loci were further analyzed by the following criteria: target sub-domains were identified as unique sequences with (1) 100% homology between the primate (RheMac3) and human (hg38) genomes, and (2) no overlap with coding mRNA. The latter criteria excluded chromosome 16p12.1 as a GSH locus because it overlaps with multiple mRNAs.

The following sites identified by the analysis are 100% homologous between the human genome and the rhesus genome.

TABLE 2
Sites Identified by Analysis that are 100% Homologous
between the Human Genome and the Rhesus Genome

| Location on the Human Genome | Location on the Rhesus Genome |
|---|---|
| chr11: 67812226-67812252 | chr20: 5298821-5298847 |
| chr11: 67812280-67812306 | chr20: 5298873-5298899 |
| chr11: 67812349-67812375 | chr20: 5298938-5298964 |
| chr11: 67812179-67812205 | chr20: 5298774-5298800 |
| chr11: 67812443-67812469 | chr20: 5299024-5299050 |
| chr11: 67931439-67931465 | chr20: 5455480-5455506 |
| chr11: 67931473-67931499 | chr20: 5455446-5455472 |
| chr11: 67931516-67931542 | chr20: 5455403-5455429 |
| chr11: 67931362-67931388 | chr20: 5455557-5455583 |

These areas of chromosome 11q13.2 represent universal HSC safe harbor loci sites. The following sites also demonstrated permissiveness to genetic modification without adverse biological consequences, even under selective pressure in vivo and represent GSH sites: chr11:67523429-67533593; chr11:67681215-67741765; chr11:67805337-67845629; chr11:67895738-67941098; chr5:66425982-66457233; chr8: 28980753-29006178; chr16: 28151114-28175716; chr1: 39189118-39214131; chr17: 2149700-2174592; chr14: 35658075-35685512; chr18: 9198556-9223041; chr5: 140463887-140488886; chr11: 68563075-68588007; chr2: 43459415-43484174; chr11: 68517649-68542970; chr1: 8600474-8624530; chr12: 50609483-50635221; chr16: 28175717-28199134; chr17: 63329602-63353111; chr1: 107643312-107672400; chr17: 65870579-65895504; chr2: 224533608-224559225; chr14: 22272733-22296704; and chr15: 50094713-50119187. In particular embodiments, chr11:67681215-67741765, chr11:67805337-67845629, and/or chr11:67895738-67941098 are targeted for genetic modification.

Universal HSC safe harbor window loci on chr11 that are particularly relevant for gene editing (as described in more detail in relation to gene editing below) include: 67935219-67935243; 67911598-67911622; 67939901-67939925; 67927758-67927782; 67917930-67917954; 67918042-67918066; 67931473-67931497; 67936715-67936739; 67921126-7921150; 67914940-67914964; 67928284-67928308; 67936068-67936092; 67922372-67922396; 67811255-67811279; 67840351-67840375; 67821576-67821600; 67827279-67827303; 67822563-7822587; 67823914-67823938; 67818875-67818899; 67811907-67811931; 67811630-67811654; 7836644-67836668; 67806757-67806781; 67823923-67823947; 67841379-67841403; 67808086-7808110; 67823903-67823927; 67686904-67686928; 67692610-67692634; 67692462-67692486; 67692618-67692642; 67705405-67705429; 67686651-67686675; 67686788-67686812; 67684033-7684057; 67681565-67681589; 67704652-67704676; 67689328-67689352; 67688546-67688570; 67693464-67693488; 67682343-67682367; 67689948-67689972; 67684785-67684809; 67684738-67684762; 67684260-67684284; 67684173-67684197; 67687315-67687339; 67682671-67682695; 67691534-67691558; 67690743-67690767; 67693746-67693770; 67690174-67690198; 67692535-67692559; 67687605-67687629; 67694747-67694771; 67681441-67681465; 67691508-67691532; 67692057-67692081; 67692573-67692597; 67690331-67690355; 67697247-67697271; 67695745-67695769; 67695241-67695265; 67691931-67691955; 67691017-67691041; 67694689-67694713; 67721934-67721958; 67696164-67696188; 67736715-67736739; 67681498-67681522; 67690926-67690950; 67694271-67694295; 67682715-67682739; 67694107-67694131; 67692129-67692153; 67721153-67721177; 67726733-67726757; 67694551-67694575; 67684767-67684791; 67686717-67686741; 67692858-67692882; 67694890-67694914; 7706343-67706367; 67681596-67681620; 67684153-67684177; 67690025-67690049; 67691225-67691249; 67692361-67692385; 67692291-67692315; 67684752-67684776; 67690917-67690941; 67695354-67695378; 67685964-67685988; 67690852-67690876; 67698221-67698245; 67713445-67713469; 67693965-67693989; 67689830-67689854; 67690151-67690175; 67718079-67718103; 67692663-67692687; 67684143-67684167; 67702560-67702584; 67689807-67689831; 67734305-67734329; 67691410-67691434; 67691162-67691186; 67702695-67702719; 67689612-67689636; 67697284-67697308; 67691567-67691591; 67685635-67685659; 67689900-67689924; 67696035-67696059; 67687462-67687486; 67689863-67689887; 67690831-67690855; 67696956-67696980; 67703966-67703990; 67692382-67692406; 67693741-67693765; 67682707-67682731; 67689891-67689915; 67695833-67695857; 67689800-67689824; 67693566-67693590; 67681587-67681611; 67702113-67702137; 67701288-67701312; 67689761-67689785; 67723825-67723849; 67686892-67686916; 67698097-67698121; 67687614-67687638; 67703251-67703275; 67690109-67690133; 67719750-67719774; 67691762-67691786; 67691654-67691678; 67695445-67695469; 67694579-67694603; 67693002-67693026; 67731932-67731956; 67689608-67689632; 67691726-67691750; 67704995-67705019; 67694095-67694119; 67688285-67688309; 67692918-67692942; 67735442-67735466; 67694119-67694143; 67694791-67694815; 67695843-67695867; 67695032-67695056; 67703734-67703758; 67690809-67690833; 67697085-67697109; 67690629-67690653; 67701642-67701666; 67693639-67693663; 67703876-67703900; 67690054-67690078; 67695062-67695086; 67689878-67689902; 67696347-67696371; 67694806-67694830; 67690245-67690269; 67695377-67695401; 67694295-67694319; 67705602-67705626; 67693729-67693753; 67694696-67694720; 67694318-67694342; 67697768-67697792; 67694989-67695013; 67687551-67687575; 67694309-67694333; 67693926-67693950; 67693602-67693626; 67693896-67693920; 67718020-67718044; 67700346-67700370; 67696171-67696195; 67729142-67729166; 67684112-67684136; 67693375-67693399; 67691807-67691831; 67700198-67700222; 67697504-67697528; 67701370-67701394; 67703871-67703895; 67683323-67683347; and 67690737-67690761. These sites represent SEQ ID NOs. 1-194 as provided in Table 3 below.

While GSH loci described herein are ideally suited for genetic manipulation in HSC including a subset for CD34+ cells, CD34⁺CD45RA⁻CD90⁺ HSC), other appropriate blood cells types include hematopoietic progenitor cells (HPC), hematopoietic stem and progenitor cell (HSPCs), T cells, natural killer (NK) cells, B cells, macrophages, monocytes, mesenchymal stem cells (MSC), white blood cell (WBC), mononuclear cell (MNC), endothelial cells (EC), stromal cells, and bone marrow fibroblasts. These cell types can collectively be referred to as “blood cells”.

(II) Gene Editing Systems and Components to Target and Modify GSH Sites. Identification of the above-described GSH and more rigorously defined universal HSC safe harbor loci allows targeting with gene editing systems, greatly increasing the safety of genetic therapies. Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. When insertion of a therapeutic nucleic acid sequence is intended, the systems also include a homology-directed repair template (which can include homology arms) associated with the therapeutic nucleic acid sequence. As detailed further below, however, different gene editing systems can adopt different components and configurations while maintaining the ability to precisely target, cut, and modify selected genomic sites.

Particular embodiments utilize zinc finger nucleases (ZFNs) as gene editing agents. ZFNs are a class of site-specific nucleases engineered to bind and cleave DNA at specific positions. ZFNs are used to introduce double strand breaks (DSBs) at a specific site in a DNA sequence which enables the ZFNs to target unique sequences within a genome in a variety of different cells. Moreover, subsequent to double-stranded breakage, homology-directed repair (HDR) or non-homologous end joining (NHEJ) takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, FokI endonuclease. The FokI domain functions as a dimer requiring two constructs with unique DNA binding domains for sites on the target sequence. The FokI cleavage domain cleaves within a five or six base pair spacer sequence separating the two inverted half-sites.

For additional information regarding ZFNs, see Kim, et al. Proceedings of the National Academy of Sciences of the United States of America 93, 1156-1160 (1996); Wolfe, et al. Annual review of biophysics and biomolecular structure 29, 183-212 (2000); Bibikova, et al. Science 300, 764 (2003); Bibikova, et al. Genetics 161, 1169-1175 (2002); Miller, et al. The EMBO journal 4, 1609-1614 (1985); and Miller, et al. Nature biotechnology 25, 778-785 (2007)].

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. For additional information regarding TALENs, see Boch, et al. Science 326, 1509-1512 (2009); Moscou, & Bogdanove, Science 326, 1501 (2009); Christian, et al. Genetics 186, 757-761 (2010); and Miller, et al. Nature biotechnology 29, 143-148 (2011).

Particular embodiments utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

In particular embodiments, GSH can be targeted using CRISPR gene editing systems. The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a cas (CRISPR-associated) nuclease. A CRISPR-Cas system including an RNA having a sequence that can hybridize to the foreign genetic elements and Cas nuclease can then recognize and cut these exogenous genetic elements in the genome.

A CRISPR-Cas system does not require the generation of customized proteins to target specific sequences, but rather a single Cas enzyme can be programmed by a short guide RNA molecule (crRNA) to recognize a specific DNA target. The CRISPR-Cas systems of bacterial and archaeal adaptive immunity show extreme diversity of protein composition and genomic loci architecture. The CRISPR-Cas system loci have more than 50 gene families and there are no strictly universal genes, indicating fast evolution and extreme diversity of loci architecture. So far, adopting a multi-pronged approach, there is comprehensive cas gene identification of 395 profiles for 93 Cas proteins. Classification includes signature gene profiles plus signatures of locus architecture. A new classification of CRISPR-Cas systems is proposed in which these systems are broadly divided into two classes, Class 1 with multi-subunit effector complexes and Class 2 with single-subunit effector modules exemplified by the Cas9 protein. Efficient gene editing in human CD34+ cells using electroporation of CRISPR/Cas9 mRNA and single-stranded oligodeoxyribonucleotide (ssODN) as a donor template for HDR has been demonstrated. De Ravin et al. Sci Transl Med. 2017; 9(372): eaah3480. Novel effector proteins associated with Class 2 CRISPR-Cas systems may be developed as powerful genome engineering tools and the prediction of putative novel effector proteins and their engineering and optimization is important. In addition to the Class 1 and Class 2 CRISPR-Cas systems, more recently a putative Class 2, Type V CRISPR-Cas class exemplified by Cpf1 has been identified Zetsche et al. (2015) Cell 163(3): 759-771.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR. Moreover, staggered DSBs with sticky ends permit orientation-specific donor template insertion, which is advantageous in non-dividing cells.

Three windows of identified GSH sites on chromosome 11q13.2 were searched for Cpf1 target sites that contained the most preferred PAM sequence (TTTA) and an adjacent 21 of DNA which was completely unique to the human genome. A total of 194 target cut sites were identified by these criteria and are listed in Table 3. Each of these identified sequences provides a beneficial site to specifically target for gene therapy. The disclosed nucleic acid sequences are shown using standard letter abbreviations for nucleotide bases, as defined in 37 C.F.R. 1.822. Only one strand of each nucleic acid sequence is shown, but the complementary strand is understood as included by any reference to the displayed strand in the following Table 3 and/or as provided herein:

TABLE 3
Universal HSC Safe Harbor Loci with Cpf1
Target Sites that Contain the Most
Preferred PAM Sequence with 21 Adjacent
Base Pairs that are Unique to the Human Genome

| | | Number of | | |
|---|---|---|---|---|
| | | Sequences | | |
| | | Identified | | |
| | | in the | | |
| Universal | | Human | | |
| HSC | | Genome | | |
| Safe | | (hg38)* | | |
| Harbor‡ | Cpf1 Target | | One | Two |
| Window on | Sequence | Exact | bp | bp |
| chr11: | (TTTA = PAM†) | Match | Off | Off |
| 67935219- | TTTAAAAAAAGTCTTCATAA | 1 | 0 | 0 |
| 67935243 | TAAAA (SEQ ID NO: 1) | | | |
| 67911598- | TTTACTTCTGTGCACCCAT | 1 | 0 | 0 |
| 67911622 | AGACTC (SEQ ID NO: 2) | | | |
| 67939901- | TTTAACTCTGCCATGGGTG | 1 | 0 | 0 |
| 67939925 | CCAGGA (SEQ ID NO: 3) | | | |
| 67927758- | TTTAGCCACTGTGAGAAAC | 1 | 0 | 0 |
| 67927782 | AGTTTA (SEQ ID NO: 4) | | | |
| 67917930- | TTTATAAGGCTAAGTAGTA | 1 | 0 | 0 |
| 67917954 | TTCTAT (SEQ ID NO: 5) | | | |
| 67918042- | TTTATCCACTAATTGTTGAT | 1 | 0 | 0 |
| 67918066 | GGGCA (SEQ ID NO: 6) | | | |
| 67931473- | TTTATTTTCTTTTTGGTAAG | 1 | 0 | 0 |
| 67931497 | AAGGA (SEQ ID NO: 7) | | | |
| 67936715- | TTTAAAATAATCGTCATTCT | 1 | 0 | 0 |
| 67936739 | TTTTG (SEQ ID NO: 8) | | | |
| 67921126- | TTTATATTAATCCTTGTATG | 1 | 0 | 0 |
| 67921150 | TCTGA (SEQ ID NO: 9) | | | |
| 67914940- | TTTAAATGGATAAGTTAGG | 1 | 0 | 0 |
| 67914964 | CTGGGC (SEQ ID NO: 10) | | | |
| 67928284- | TTTACACAAACTGTACTTGT | 1 | 0 | 0 |
| 67928308 | AGGTA (SEQ ID NO: 11) | | | |
| 67936068- | TTTAGTGAGGAAAAACTAC | 1 | 0 | 0 |
| 67936092 | ATTTAT (SEQ ID NO: 12) | | | |
| 67922372- | TTTAAAAAGGGAAAATTAG | 1 | 0 | 0 |
| 67922396 | GGAGAA (SEQ ID NO: 13) | | | |
| 67811255- | TTTACTAAATGACCTCTTC | 1 | 0 | 0 |
| 67811279 | GCCAAA (SEQ ID NO: 14) | | | |
| 67840351- | TTTAGTGATATGAAAAGAA | 1 | 0 | 0 |
| 67840375 | CCATGC (SEQ ID NO: 15) | | | |
| 67821576- | TTTAAATTTTCCAGTGTCTC | 1 | 0 | 0 |
| 67821600 | AGTGT (SEQ ID NO: 16) | | | |
| 67827279- | TTTAAGCCTGAAAACCTAA | 1 | 0 | 0 |
| 67827303 | AAAATG (SEQ ID NO: 17) | | | |
| 67822563- | TTTAAAAATTAGCTGCGTT | 1 | 0 | 0 |
| 67822587 | GGTGGT (SEQ ID NO: 18) | | | |
| 67823914- | TTTAGCTGCTTTAAAATGT | 1 | 0 | 0 |
| 67823938 | GAACTC (SEQ ID NO: 19) | | | |
| 67818875- | TTTAATCTCAAGTCACTTCT | 1 | 0 | 0 |
| 67818899 | CTAGG (SEQ ID NO: 20) | | | |
| 67811907- | TTTAAACGCTCGTTAGATC | 1 | 0 | 0 |
| 67811931 | ACTGGA (SEQ ID NO: 21) | | | |
| 67811630- | TTTAAGGCAGGAATCAGGG | 1 | 0 | 0 |
| 67811654 | TGCCAT (SEQ ID NO: 22) | | | |
| 67836644- | TTTAAAAAAATCCCAGGTG | 1 | 0 | 0 |
| 67836668 | ACCCTA (SEQ ID NO: 23) | | | |
| 67806757- | TTTAATCTTGCAGGGACAG | 1 | 0 | 0 |
| 67806781 | GAAGGA (SEQ ID NO: 24) | | | |
| 67823923- | TTTAAAATGTGAACTCCAA | 1 | 0 | 0 |
| 67823947 | ATCAGG (SEQ ID NO: 25) | | | |
| 67841379- | TTTATTATTTCGTTGTTTTT | 1 | 0 | 0 |
| 67841403 | CCTCT (SEQ ID NO: 26) | | | |
| 67808086- | TTTATCTTGGTGTTTGAACT | 1 | 0 | 0 |
| 67808110 | CGGAT (SEQ ID NO: 27) | | | |
| 67823903- | TTTACTGCCCATTTAGCTG | 1 | 0 | 0 |
| 67823927 | CTTTAA (SEQ ID NO: 28) | | | |
| 67686904- | TTTAACTTGATTACCTTATA | 1 | 0 | 0 |
| 67686928 | GTCAA (SEQ ID NO: 29) | | | |
| 67692610- | TTTATATATTTAACTTCTGT | 1 | 0 | 0 |
| 67692634 | ATTTT (SEQ ID NO: 30) | | | |
| 67692462- | TTTATCTGCTATAGCTAACA | 1 | 0 | 0 |
| 67692486 | AATTT (SEQ ID NO: 31) | | | |
| 67692618- | TTTAACTTCTGTATTTTCTA | 1 | 0 | 0 |
| 67692642 | AAACT (SEQ ID NO: 32) | | | |
| 67705405- | TTTATAACACACCAGCATC | 1 | 0 | 0 |
| 67705429 | AGTTAC (SEQ ID NO: 33) | | | |
| 67686651- | TTTATTATCTTTCCTGTTTT | 1 | 0 | 0 |
| 67686675 | CTAAT (SEQ ID NO: 34) | | | |
| 67686788- | TTTAATATTAACATTTGCTA | 1 | 0 | 0 |
| 67686812 | ATTTC (SEQ ID NO: 35) | | | |
| 67684033- | TTTAAAAGGCCAGATGTCA | 1 | 0 | 0 |
| 67684057 | ATTCAG (SEQ ID NO: 36) | | | |
| 67681565- | TTTATGCAGATAGATATATA | 1 | 0 | 0 |
| 67681589 | TTTTT (SEQ ID NO: 37) | | | |
| 67704652- | TTTAATCACTGGATCATGG | 1 | 0 | 0 |
| 67704676 | ACCTAA (SEQ ID NO: 38) | | | |
| 67689328- | TTTAGTGTTTCTTAGAAACT | 1 | 0 | 0 |
| 67689352 | TAACA (SEQ ID NO: 39) | | | |
| 67688546- | TTTACAGCAGTGCCATTCA | 1 | 0 | 0 |
| 67688570 | CAATGG (SEQ ID NO: 40) | | | |
| 67693464- | TTTATAGGCCCAGGTCTAG | 1 | 0 | 0 |
| 67693488 | ATCTGG (SEQ ID NO: 41) | | | |
| 67682343- | TTTAGCCCTATCTTATCCAT | 1 | 0 | 0 |
| 67682367 | ATGGA (SEQ ID NO: 42) | | | |
| 67689948- | TTTATGTCTAGTACTTAGG | 1 | 0 | 0 |
| 67689972 | GCAGTA (SEQ ID NO: 43) | | | |
| 67684785- | TTTACAATTAATTGTAGTTC | 1 | 0 | 0 |
| 67684809 | TTTGA (SEQ ID NO: 44) | | | |
| 67684738- | TTTATTTTCTTAGATTTACT | 1 | 0 | 0 |
| 67684762 | CTGTA (SEQ ID NO: 45) | | | |
| 67684260- | TTTAAAAACTGGGTTTACA | 1 | 0 | 0 |
| 67684284 | AATAAA (SEQ ID NO: 46) | | | |
| 67684173- | TTTAAACATTTTGACTGTAG | 1 | 0 | 0 |
| 67684197 | CCATT (SEQ ID NO: 47) | | | |
| 67687315- | TTTATGACTGTTTCATGTGT | 1 | 0 | 0 |
| 67687339 | GCTCA (SEQ ID NO: 48) | | | |
| 67682671- | TTTATAACCTCACCTTTGG | 1 | 0 | 0 |
| 67682695 | CTTTTA (SEQ ID NO: 49) | | | |
| 67691534- | TTTAGAAGTCCATATAAGG | 1 | 0 | 0 |
| 67691558 | GGATGC (SEQ ID NO: 50) | | | |
| 67690743- | TTTACTGTTAATTAGTCCTT | 1 | 0 | 0 |
| 67690767 | GCTTA (SEQ ID NO: 51) | | | |
| 67693746- | TTTATCTATGAACCTCATAG | 1 | 0 | 0 |
| 67693770 | GTCCT (SEQ ID NO: 52) | | | |
| 67690174- | TTTACTATAGTTAATTGGAA | 1 | 0 | 0 |
| 67690198 | CACTT (SEQ ID NO: 53) | | | |
| 67692535- | TTTAACGTTAAATCTCTTTC | 1 | 0 | 0 |
| 67692559 | TAACA (SEQ ID NO: 54) | | | |
| 67687605- | TTTATCTCCTTTAAATTCCC | 1 | 0 | 0 |
| 67687629 | ATGTT (SEQ ID NO: 55) | | | |
| 67694747- | TTTAGTCCAAATAAAGCAA | 1 | 0 | 0 |
| 67694771 | CAATAC (SEQ ID NO: 56) | | | |
| 67681441- | TTTATCCATTCCCAACCAC | 1 | 0 | 0 |
| 67681465 | AAAGAA (SEQ ID NO: 57) | | | |
| 67691508- | TTTAATTTCTCCACTTGATT | 1 | 0 | 0 |
| 67691532 | AACTT (SEQ ID NO: 58) | | | |
| 67692057- | TTTAGCCAAAGGACATGCC | 1 | 0 | 0 |
| 67692081 | TAAAAT (SEQ ID NO: 59) | | | |
| 67692573- | TTTAGGAATTAATAAAAATT | 1 | 0 | 0 |
| 67692597 | GTATA (SEQ ID NO: 60) | | | |
| 67690331- | TTTAATGCATCCATAAACA | 1 | 0 | 0 |
| 67690355 | GAACTG (SEQ ID NO: 61) | | | |
| 67697247- | TTTAGGGTGGTTCTCCTGG | 1 | 0 | 0 |
| 67697271 | GATTTT (SEQ ID NO: 62) | | | |
| 67695745- | TTTATCCCTTACGCATGAG | 1 | 0 | 0 |
| 67695769 | GTCCCT (SEQ ID NO: 63) | | | |
| 67695241- | TTTACACAAGCAACACCAG | 1 | 0 | 0 |
| 67695265 | CTGCAG (SEQ ID NO: 64) | | | |
| 67691931- | TTTAAATACTTGACAAAAAA | 1 | 0 | 0 |
| 67691955 | GATTG (SEQ ID NO: 65) | | | |
| 67691017- | TTTAAAAGGGGTCTCTACT | 1 | 0 | 0 |
| 67691041 | AAATCT (SEQ ID NO: 66) | | | |
| 67694689- | TTTAATCTTTATCTGACCTA | 1 | 0 | 0 |
| 67694713 | AATTT (SEQ ID NO: 67) | | | |
| 67721934- | TTTATAAAGAAATTCCGCA | 1 | 0 | 0 |
| 67721958 | AGAACT (SEQ ID NO: 68) | | | |
| 67696164- | TTTATGATTTAAAGGGGAA | 1 | 0 | 0 |
| 67696188 | GCTTTG (SEQ ID NO: 69) | | | |
| 67736715- | TTTAATGCTTTCAACATCGA | 1 | 0 | 0 |
| 67736739 | TTGCT (SEQ ID NO: 70) | | | |
| 67681498- | TTTACGGAGAGGATACAAA | 1 | 0 | 0 |
| 67681522 | GATCCT (SEQ ID NO: 71) | | | |
| 67690926- | TTTAAGATCTTCCTATAATT | 1 | 0 | 0 |
| 67690950 | ATAGC (SEQ ID NO: 72) | | | |
| 67694271- | TTTAATTTTTGTTGGAGTCT | 1 | 0 | 0 |
| 67694295 | TTTCT (SEQ ID NO: 73) | | | |
| 67682715- | TTTATTTTTTAAAACCAGAA | 1 | 0 | 0 |
| 67682739 | CATTT (SEQ ID NO: 74) | | | |
| 67694107- | TTTAGTTCTCTTTTTATACT | 1 | 0 | 0 |
| 67694131 | CCAAA (SEQ ID NO: 75) | | | |
| 67692129- | TTTACTAAGAATAGTGTAG | 1 | 0 | 0 |
| 67692153 | GGGTTA (SEQ ID NO: 76) | | | |
| 67721153- | TTTACTATTTCCCATCTTAT | 1 | 0 | 0 |
| 67721177 | GTATA (SEQ ID NO: 77) | | | |
| 67726733- | TTTAGCAGGTGTCTTGATC | 1 | 0 | 0 |
| 67726757 | CCCCTT (SEQ ID NO: 78) | | | |
| 67694551- | TTTATTTTCTAGTCCCCCTT | 1 | 0 | 0 |
| 67694575 | TGATC (SEQ ID NO: 79) | | | |
| 67684767- | TTTAAAATCATCTTATTGTT | 1 | 0 | 0 |
| 67684791 | TACAA (SEQ ID NO: 80) | | | |
| 67686717- | TTTAGAGAGATATATTTTCC | 1 | 0 | 0 |
| 67686741 | TCTAG (SEQ ID NO: 81) | | | |
| 67692858- | TTTAAAAGTGGTCACAAGT | 1 | 0 | 0 |
| 67692882 | GGGGGA (SEQ ID NO: 82) | | | |
| 67694890- | TTTACTAGAAACCTTTCCC | 1 | 0 | 0 |
| 67694914 | ATATTG (SEQ ID NO: 83) | | | |
| 67706343- | TTTATTGCTCTGTAACAGAT | 1 | 0 | 0 |
| 67706367 | TACCA (SEQ ID NO: 84) | | | |
| 67681596- | TTTACCAGCCATCTTAGAA | 1 | 0 | 0 |
| 67681620 | CAAATT (SEQ ID NO: 85) | | | |
| 67684153- | TTTACAGAATTCGCTTTCC | 1 | 0 | 0 |
| 67684177 | CTTTAA (SEQ ID NO: 86) | | | |
| 67690025- | TTTATTAAGCTAAACCTAG | 1 | 0 | 0 |
| 67690049 | GTACAA (SEQ ID NO: 87) | | | |
| 67691225- | TTTAGGATCACCTTAACTT | 1 | 0 | 0 |
| 67691249 | GGTGAG (SEQ ID NO: 88) | | | |
| 67692361- | TTTACTATTTCCCCTGGAG | 1 | 0 | 0 |
| 67692385 | TCTTTA (SEQ ID NO: 89) | | | |
| 67692291- | TTTAATAAGTCTTTTGATTA | 1 | 0 | 0 |
| 67692315 | CAGGC (SEQ ID NO: 90) | | | |
| 67684752- | TTTACTCTGTAGCTTTTTAA | 1 | 0 | 0 |
| 67684776 | AATCA (SEQ ID NO: 91) | | | |
| 67690917- | TTTACTTCTTTTAAGATCTT | 1 | 0 | 0 |
| 67690941 | CCTAT (SEQ ID NO: 92) | | | |
| 67695354- | TTTACTTTGCTCTGTGAAC | 1 | 0 | 0 |
| 67695378 | AGAGTT (SEQ ID NO: 93) | | | |
| 67685964- | TTTAATTCCTGTTTCATTTT | 1 | 0 | 0 |
| 67685988 | CCCAT (SEQ ID NO: 94) | | | |
| 67690852- | TTTAGGGCGTGACTGTGAA | 1 | 0 | 0 |
| 67690876 | TAACTC (SEQ ID NO: 95) | | | |
| 67698221- | TTTATTCAATTTCTCCTAAG | 1 | 0 | 0 |
| 67698245 | TCTGC (SEQ ID NO: 96) | | | |
| 67713445- | TTTAAAAATATTTAGCAACT | 1 | 0 | 0 |
| 67713469 | GGGAC (SEQ ID NO: 97) | | | |
| 67693965- | TTTACGTTCCCAGATCGTA | 1 | 0 | 0 |
| 67693989 | TTTCTT (SEQ ID NO: 98) | | | |
| 67689830- | TTTAGTTCATGGCAAGCAA | 1 | 0 | 0 |
| 67689854 | GTCATT (SEQ ID NO: 99) | | | |
| 67690151- | TTTAGGCCACCAATTGGGG | 1 | 0 | 0 |
| 67690175 | GCATTT (SEQ ID NO: 100) | | | |
| 67718079- | TTTACCAACCATCACTGCC | 1 | 0 | 0 |
| 67718103 | ATCGTC (SEQ ID NO: 101) | | | |
| 67692663- | TTTAACCCCAGAAACTGTT | 1 | 0 | 0 |
| 67692687 | AATTCC (SEQ ID NO: 102) | | | |
| 67684143- | TTTATAGTTATTTACAGAAT | 1 | 0 | 0 |
| 67684167 | TCGCT (SEQ ID NO: 103) | | | |
| 67702560- | TTTATTTGTGCAACAATGG | 1 | 0 | 0 |
| 67702584 | GGAATT (SEQ ID NO: 104) | | | |
| 67689807- | TTTAATAAAGCAATAGGAA | 1 | 0 | 0 |
| 67689831 | GACGTT (SEQ ID NO: 105) | | | |
| 67734305- | TTTAAGTCACAGGGGTGTA | 1 | 0 | 0 |
| 67734329 | GACCCT (SEQ ID NO: 106) | | | |
| 67691410- | TTTAGTGACCACCCTACTC | 1 | 0 | 0 |
| 67691434 | TATTGT (SEQ ID NO: 107) | | | |
| 67691162- | TTTAATAGAATAGCCTCATA | 1 | 0 | 0 |
| 67691186 | TTTTA (SEQ ID NO: 108) | | | |
| 67702695- | TTTAACCACTCCCACTCCC | 1 | 0 | 0 |
| 67702719 | AATTAC (SEQ ID NO: 109) | | | |
| 67689612- | TTTAGATGGAACTAGCATT | 1 | 0 | 0 |
| 67689636 | CCACAA (SEQ ID NO: 110) | | | |
| 67697284- | TTTAAAAGTAGCAGCTTAA | 1 | 0 | 0 |
| 67697308 | GCCAGA (SEQ ID NO: 111) | | | |
| 67691567- | TTTAGTTAATTTCTTATATA | 1 | 0 | 0 |
| 67691591 | AGAGC (SEQ ID NO: 112) | | | |
| 67685635- | TTTAAGGTAGATCTGTGCA | 1 | 0 | 0 |
| 67685659 | GGGGGA (SEQ ID NO: 113) | | | |
| 67689900- | TTTACTCCTCCCGAAGAGG | 1 | 0 | 0 |
| 67689924 | ATGGAT (SEQ ID NO: 114) | | | |
| 67696035- | TTTAACATAGATATTGAAGT | 1 | 0 | 0 |
| 67696059 | CAGAG (SEQ ID NO: 115) | | | |
| 67687462- | TTTATCATATTACTATTTTG | 1 | 0 | 0 |
| 67687486 | CCAGT (SEQ ID NO: 116) | | | |
| 67689863- | TTTAGTTACATGATTTTTAA | 1 | 0 | 0 |
| 67689887 | GAGTT (SEQ ID NO: 117) | | | |
| 67690831- | TTTACCTGGTTCTGTAAATA | 1 | 0 | 0 |
| 67690855 | TTTTA (SEQ ID NO: 118) | | | |
| 67696956- | TTTATTATGTGAGTGATAAA | 1 | 0 | 0 |
| 67696980 | TTTGA (SEQ ID NO: 119) | | | |
| 67703966- | TTTACACCCCCCACCCCCG | 1 | 0 | 0 |
| 67703990 | AGGCCT (SEQ ID NO: 120) | | | |
| 67692382- | TTTAACTGAACATGTGTTG | 1 | 0 | 0 |
| 67692406 | GAGGAA (SEQ ID NO: 121) | | | |
| 67693741- | TTTAATTTATCTATGAACCT | 1 | 0 | 0 |
| 67693765 | CATAG (SEQ ID NO: 122) | | | |
| 67682707- | TTTATTTTTTTATTTTTTAAA | 1 | 0 | 0 |
| 67682731 | ACCA (SEQ ID NO: 123) | | | |
| 67689891- | TTTATCCTTTTTACTCCTCC | 1 | 0 | 0 |
| 67689915 | CGAAG (SEQ ID NO: 124) | | | |
| 67695833- | TTTAAACTTTTTTAAATAGG | 1 | 0 | 0 |
| 67695857 | TAAAG (SEQ ID NO: 125) | | | |
| 67689800- | TTTAATTTTTAATAAAGCAA | 1 | 0 | 0 |
| 67689824 | TAGGA (SEQ ID NO: 126) | | | |
| 67693566- | TTTACATATAGTTTTTGAGC | 1 | 0 | 0 |
| 67693590 | ClIII (SEQ ID NO: 127) | | | |
| 67681587- | TTTAAAAGTTTTACCAGCC | 1 | 0 | 0 |
| 67681611 | ATCTTA (SEQ ID NO: 128) | | | |
| 67702113- | TTTATTTGGGCTATTTGCC | 1 | 0 | 0 |
| 67702137 | AAACAG (SEQ ID NO: 129) | | | |
| 67701288- | TTTAACTATGGTTCCTTTAA | 1 | 0 | 0 |
| 67701312 | ATCAG (SEQ ID NO: 130) | | | |
| 67689761- | TTTATAAGGGGACAATCCA | 1 | 0 | 0 |
| 67689785 | ACATCT (SEQ ID NO: 131) | | | |
| 67723825- | TTTATTCGGACCCGTGCTA | 1 | 0 | 0 |
| 67723849 | CAACTT (SEQ ID NO: 132) | | | |
| 67686892- | TTTATTATCCATTTTAACTT | 1 | 0 | 0 |
| 67686916 | GATTA (SEQ ID NO: 133) | | | |
| 67698097- | TTTATCAGTTGTCCAATTTG | 1 | 0 | 0 |
| 67698121 | TGGTG (SEQ ID NO: 134) | | | |
| 67687614- | TTTAAATTCCCATGTTGCAA | 1 | 0 | 0 |
| 67687638 | CCCTA (SEQ ID NO: 135) | | | |
| 67703251- | TTTAATAATTTTTCTACTTA | 1 | 0 | 0 |
| 67703275 | TACTT (SEQ ID NO: 136) | | | |
| 67690109- | TTTACCCAGTGGGTAAAAT | 1 | 0 | 0 |
| 67690133 | GATCTA (SEQ ID NO: 137) | | | |
| 67719750- | TTTAGGTGTATGATACTTTT | 1 | 0 | 0 |
| 67719774 | AGTGC (SEQ ID NO: 138) | | | |
| 67691762- | TTTAGGTTCTACATATTGAA | 1 | 0 | 0 |
| 67691786 | GCTTT (SEQ ID NO: 139) | | | |
| 67691654- | TTTAAGTTCTTGTTTGGTTC | 1 | 0 | 0 |
| 67691678 | GGGGC (SEQ ID NO: 140) | | | |
| 67695445- | TTTATTCATCACAACAGGT | 1 | 0 | 0 |
| 67695469 | AAGTCC (SEQ ID NO: 141) | | | |
| 67694579- | TTTAAAGGATAAAGAATAAT | 1 | 0 | 0 |
| 67694603 | ATAGG (SEQ ID NO: 142) | | | |
| 67693002- | TTTATTTTCACATCCACAGC | 1 | 0 | 0 |
| 67693026 | TCCTA (SEQ ID NO: 143) | | | |
| 67731932- | TTTAGTCCCCGCATCGTGT | 1 | 0 | 0 |
| 67731956 | GGGGGG (SEQ ID NO: 144) | | | |
| 67689608- | TTTATTTAGATGGAACTAG | 1 | 0 | 0 |
| 67689632 | CATTCC (SEQ ID NO: 145) | | | |
| 67691726- | TTTATCTGCACTTATTAAAT | 1 | 0 | 0 |
| 67691750 | GGCCT (SEQ ID NO: 146) | | | |
| 67704995- | TTTATCCTTGGACATAATTA | 1 | 0 | 0 |
| 67705019 | AAGAA (SEQ ID NO: 147) | | | |
| 67694095- | TTTAACAGTGGCTTTAGTT | 1 | 0 | 0 |
| 67694119 | CTCTTT (SEQ ID NO: 148) | | | |
| 67688285- | TTTAGTACAGCAGCCTGAA | 1 | 0 | 0 |
| 67688309 | CTGACT (SEQ ID NO: 149) | | | |
| 67692918- | TTTAAAACGACCTGGTCTC | 1 | 0 | 0 |
| 67692942 | CCGCAT (SEQ ID NO: 150) | | | |
| 67735442- | TTTACTCTGTGACAATATAT | 1 | 0 | 0 |
| 67735466 | TCTAT (SEQ ID NO: 151) | | | |
| 67694119- | TTTATACTCCAAACTTCAGA | 1 | 0 | 0 |
| 67694143 | CCCAG (SEQ ID NO: 152) | | | |
| 67694791- | TTTAAATTTAGTTTTTTTATT | 1 | 0 | 0 |
| 67694815 | ATCT (SEQ ID NO: 153) | | | |
| 67695843- | TTTAAATAGGTAAAGGCAG | 1 | 0 | 0 |
| 67695867 | GGAGGA (SEQ ID NO: 154) | | | |
| 67695032- | TTTAACTCCTCTTTTTCTTT | 1 | 0 | 0 |
| 67695056 | CTGGA (SEQ ID NO: 155) | | | |
| 67703734- | TTTAATTTGGGAATATTGG | 1 | 0 | 0 |
| 67703758 | GTTAAT (SEQ ID NO: 156) | | | |
| 67690809- | TTTAGAGTCAGTATAGATG | 1 | 0 | 0 |
| 67690833 | GTTTTT (SEQ ID NO: 157) | | | |
| 67697085- | TTTATGCTGGGAACCGGAG | 1 | 0 | 0 |
| 67697109 | GGCTGG (SEQ ID NO: 158) | | | |
| 67690629- | TTTAAAAGAAAAGTTAGGT | 1 | 0 | 0 |
| 67690653 | TGGTGT (SEQ ID NO: 159) | | | |
| 67701642- | TTTATGTTGTACATGCCAC | 1 | 0 | 0 |
| 67701666 | AAAAAA (SEQ ID NO: 160) | | | |
| 67693639- | TTTAGTAATGTCTGGCCAA | 1 | 0 | 0 |
| 67693663 | CTGTGA (SEQ ID NO: 161) | | | |
| 67703876- | TTTATTTCTTGTTGGGAGG | 1 | 0 | 0 |
| 67703900 | ATGAGG (SEQ ID NO: 162) | | | |
| 67690054- | TTTAATTAAGGCTTTGACT | 1 | 0 | 0 |
| 67690078 | GCATTA (SEQ ID NO: 163) | | | |
| 67695062- | TTTACACTCTTCACTCGCTT | 1 | 0 | 0 |
| 67695086 | TGTCC (SEQ ID NO: 164) | | | |
| 67689878- | TTTAAGAGTTTGATTTATCC | 1 | 0 | 0 |
| 67689902 | TTTTT (SEQ ID NO: 165) | | | |
| 67696347- | TTTAGCTATTTGTTATGGCA | 1 | 0 | 0 |
| 67696371 | GCAAC (SEQ ID NO: 166) | | | |
| 67694806- | TTTATTATCTTTCCAATACT | 1 | 0 | 0 |
| 67694830 | TTAAC (SEQ ID NO: 167) | | | |
| 67690245- | TTTAAGGCTTGTTTATTTGT | 1 | 0 | 0 |
| 67690269 | GTTTT (SEQ ID NO: 168) | | | |
| 67695377- | TTTATCTGGTCCCCGAGGC | 1 | 0 | 0 |
| 67695401 | AGTGCA (SEQ ID NO: 169) | | | |
| 67694295- | TTTAAAGAAGGATATTTAG | 1 | 0 | 0 |
| 67694319 | AATTTT (SEQ ID NO: 170) | | | |
| 67705602- | TTTAAAGGTAGGCCTCAAA | 1 | 0 | 0 |
| 67705626 | AAGAAC (SEQ ID NO: 171) | | | |
| 67693729- | TTTATTTGTTCTTTTAATTTA | 1 | 0 | 0 |
| 67693753 | TCTA (SEQ ID NO: 172) | | | |
| 67694696- | TTTATCTGACCTAAATTTTG | 1 | 0 | 0 |
| 67694720 | ACCAA (SEQ ID NO: 173) | | | |
| 67694318- | TTTAGGCTCCTGGGATTCA | 1 | 0 | 0 |
| 67694342 | CAAGAA (SEQ ID NO: 174) | | | |
| 67697768- | TTTACTGGCAAACTGGGAG | 1 | 0 | 0 |
| 67697792 | GAGAGA (SEQ ID NO: 175) | | | |
| 67694989- | TTTAACCTTAACGTGCTTG | 1 | 0 | 0 |
| 67695013 | AGGTTT (SEQ ID NO: 176) | | | |
| 67687551- | TTTATTTCTATATTTTGAGG | 1 | 0 | 0 |
| 67687575 | ACATG (SEQ ID NO: 177) | | | |
| 67694309- | TTTAGAATTTTTAGGCTCCT | 1 | 0 | 0 |
| 67694333 | GGGAT (SEQ ID NO: 178) | | | |
| 67693926- | TTTATGATTTGCTGCCAGA | 1 | 0 | 0 |
| 67693950 | ACATTT (SEQ ID NO: 179) | | | |
| 67693602- | TTTATTGATTTTTTAAATTTT | 1 | 0 | 0 |
| 67693626 | CTAA (SEQ ID NO: 180) | | | |
| 67693896- | TTTATCCCACTGCGGGTCC | 1 | 0 | 0 |
| 67693920 | TGAGCA (SEQ ID NO: 181) | | | |
| 67718020- | TTTATAATTTCCATGCTTTT | 1 | 0 | 0 |
| 67718044 | TCAGT (SEQ ID NO: 182) | | | |
| 67700346- | TTTATCTGTAATTCTGCAGA | 1 | 0 | 0 |
| 67700370 | CCCTC (SEQ ID NO: 183) | | | |
| 67696171- | TTTAAAGGGGAAGCTTTGA | 1 | 0 | 0 |
| 67696195 | AGAGGA (SEQ ID NO: 184) | | | |
| 67729142- | TTTACCTGCCGGTAGTCCT | 1 | 0 | 0 |
| 67729166 | TGGTCC (SEQ ID NO: 185) | | | |
| 67684112- | TTTACCAATGTGTTCTAAGT | 1 | 0 | 0 |
| 67684136 | TTTCA (SEQ ID NO: 186) | | | |
| 67693375- | TTTAAAAAAATAAATACTGA | 1 | 0 | 0 |
| 67693399 | CCTTG (SEQ ID NO: 187) | | | |
| 67691807- | TTTATCTTGTAGGTGGTTA | 1 | 0 | 0 |
| 67691831 | AGAACT (SEQ ID NO: 188) | | | |
| 67700198- | TTTATTTCTTTTCACGAATT | 1 | 0 | 0 |
| 67700222 | GCTGG (SEQ ID NO: 189) | | | |
| 67697504- | TTTATGTGGTGTTCAGAGC | 1 | 0 | 0 |
| 67697528 | CCCAGG (SEQ ID NO: 190) | | | |
| 67701370- | TTTATCGGTGTTATTGATG | 1 | 0 | 0 |
| 67701394 | ATCATT (SEQ ID NO: 191) | | | |
| 67703871- | TTTATTTTATTTCTTGTTGG | 1 | 0 | 0 |
| 67703895 | GAGGA (SEQ ID NO: 192) | | | |
| 67683323- | TTTAAATTGGCTATAAATCT | 1 | 0 | 0 |
| 67683347 | TTGAC (SEQ ID NO: 193) | | | |
| 67690737- | TTTACCTTTACTGTTAATTA | 1 | 0 | 0 |
| 67690761 | GTCCT (SEQ ID NO: 194) | | | |

‡GSH: loci identified by retrospective analysis of lentivirus integration sites in transplanted autologous gene modified blood stem and progenitor cells.
†PAM: protospacer adjacent motif sequence. TTTV is the denoted PAM recognition site for Cpf1; however, the strongest preference is for TTTA.
\hat{ } All sequences are depicted 5′ to 3′.

crRNA for target SEQ ID NO: 132 includes UAAUUUCUACUCUUGUAGAUUUCGGACCCGUGCUACAACUU (SEQ ID NO: 195).

crRNA for target SEQ ID NO: 108 includes UAAUUUCUACUCUUGUAGAUAUAGAAUAGCCUCAUAUUUUA (SEQ ID NO: 196)

Cpf1 crRNA target sites and PAM sites within chr11:67681215-67741765 include TTTGTGTCCCCGTTTTGGTTGGTAAAC (SEQ ID NO: 197), TTTAAAAATCAATACCGATAATAATGA (SEQ ID NO: 198), and TTTCTTAATATGAATATTAATATCGGT (SEQ ID NO: 199) (PAM sites italicized).

Cpf1 crRNA target sites and PAM sites within chr11:67805337-67845629 include TTTCCGTATCTGGAAGGGGCATCTTGG (SEQ ID NO: 200) at 67812179-67812205, TTTCCTTAGGACCGGAAGGATTACAGC (SEQ ID NO: 201) at 67812226-67812252, TTTGCCTAAAAGGCACTATGTCAAATG (SEQ ID NO: 202) at 67812280-67812306, TTTGGAGCTGTTGGCATCATGTTCCTG (SEQ ID NO: 203) at 67812349-67812375, and TTTGATTCTTTTCTATCTCAGGACAGA (SEQ ID NO: 204) (PAM sites italicized).

Cpf1 crRNA target sites and PAM sites within chr11:67895738-67941098 include: TTTATAGACATCCCACACTGTAGTTCT (SEQ ID NO: 205) at 67931362-67931388, TTTATTAATTTGAGAACCAACATAAGG (SEQ ID NO: 206) at 67931439-67931465, TTTATTTTCTTTTTGGTAAGAAGGAAC (SEQ ID NO: 207) at 67931473-67931499, and TTTCACACACACACACACACACACACA (SEQ ID NO: 208) at 67931516-67931542 (PAM sites italicized).

In particular embodiments, a Cpf1 crRNA target sequence includes TTTGGAGCTGTTGGCATCATGTTCCTG (SEQ ID NO: 203), and a crRNA for the target includes

(SEQ ID NO: 209)
UAAUUUCUACUCUUGUAGAUGAGCUGUUGGCAUCAUGUUCCUG.

In particular embodiments, a Cpf1 crRNA target sequence includes TTTATCCAAACCTCCTAAATGATAC (SEQ ID NO: 210) located at chr11:67839126-67839150, and a crRNA for the target includes:

(SEQ ID NO: 211)
UAAUUUCUACUCUUGUAGAUUCCAAACCUCCUAAAUGAUAC.

In particular embodiments, a Cpf1 crRNA target sequence includes TTTACACCCGATCCACTGGGGAGCA (SEQ ID NO: 212) located at chr3:46373915-46373939, and a crRNA for the target includes

(SEQ ID NO: 260)
UAAUUUCUACUCUUGUAGAUCACCCGAUCCACUGGGGAGCA.

crRNAs were also designed based on the following 27 nt CRISPR/Cpf1 cut site sequence: TTTTTGATTCTTTTCTATCTCAGGACA (SEQ ID NO: 213) located within chr11: 67812443-67812469.

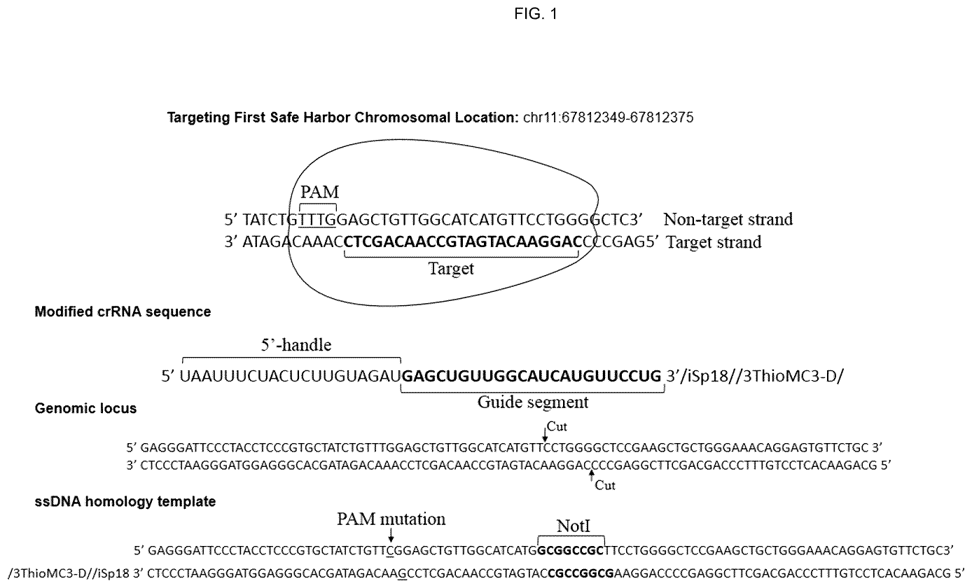

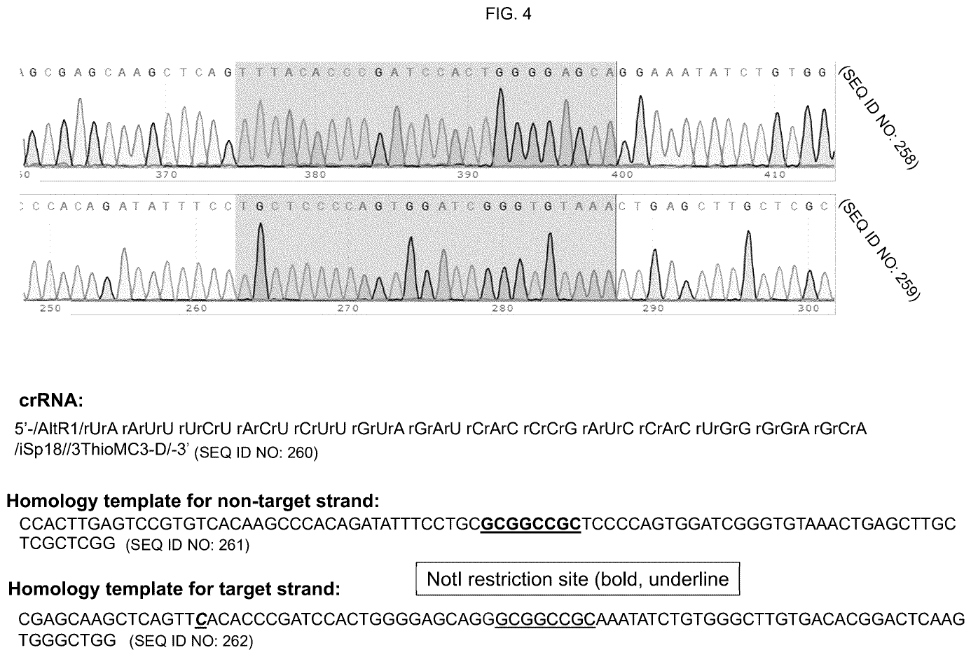

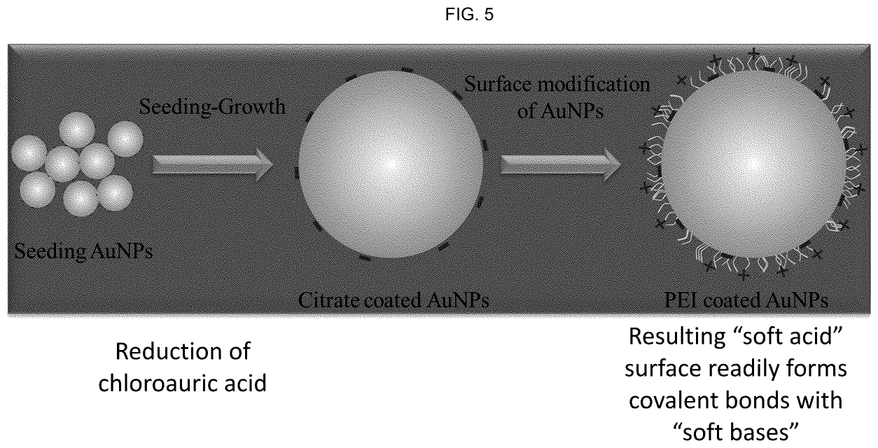

Homology-directed repair templates for HDR were also designed for nuclease-guide pairs with symmetric or asymmetric homology arms as described by Richardson et al., Nat Biotechnol. 2016; 34(3):339-44. Each donor template included homology arms (homology-directed repair template) flanking a 20 bp random DNA barcode element for clone tracking, upstream of a human phosphoglycerate kinase (PGK) promoter (e.g., SEQ ID NO: 214) driving expression of an enhanced green fluorescent protein (GFP) reporter gene (for experimental purposes, but akin to a therapeutic DNA sequence in clinical use). Humanized Cpf1 protein was synthesized by a commercial manufacturer (Aldevron), and guide RNA with two modifications, an atom oligoehtylene glycol spacer and a 3′ terminal thiol was also obtained from a commercial source (Integrated DNA Technologies). Single-stranded homology template DNA (ssODN) was also synthesized by a commercial manufacturer (Integrated DNA Technologies). For examples of such sequences, see FIGS. 1, 3, 4, and 16.

As indicated, in particular embodiments, gene editing systems to provide a genetic therapy within a GSH will include guide RNA and a nuclease. In particular embodiments, donor templates can be used, especially when performing a gain-of-function therapy or a precise loss-of-function therapy. In particular embodiments, gene editing systems include a homology-directed repair template and a therapeutic nucleic acid sequence.

All nucleic acid-based components of gene editing systems can be single stranded, double stranded, or may have mix of single stranded and double stranded regions. For example, guide RNA or a donor template may be a single-stranded DNA, a single-stranded RNA, a double-stranded DNA, or a double-stranded RNA. In particular embodiments utilizing nanoparticles described herein, the end of a nucleic acid farthest from the nanoparticle surface may be protected (e.g., from exonucleolytic degradation) by methods known to those of skill in the art. For example, one or more dideoxynucleotide residues can be added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include addition of terminal amino group(s) and the use of modified internucleotide linkages such as, for example, phosphorothioates, phosphoramidates, and O-methyl ribose or deoxyribose residues. Chemically modified mRNA can be used to increase intracellular stability, while asymmetric homology arms and phosphorothioate modification can be incorporated into the ssODN to improve HDR efficiency. In particular embodiments utilizing nanoparticles described herein, nucleic acids may be protected from electrostatic (charge-based) repulsions by, for example, addition of a charge shielding spacer. In particular embodiments, a charge shielding spacer can include an 18 atom oligoethylene glycol (OEG) spacer added to one or both ends. In particular embodiments, a charge shielding spacer can include a 10-26 atom oligoethylene glycol (OEG) spacer added to one or both ends.

Donor templates can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

In particular embodiments, a homology-directed repair template is designed to serve as a template in homologous recombination, such as within or near a target sequence nicked or cleaved by an enzyme (e.g., nuclease) of a gene editing system. A homology-directed repair template polynucleotide may be of any suitable length, such as 10, 15, 20, 25, 50, 75, 100, 150, 200, 500, 1000, 2000, 3000, 4000, 5000, or more nucleotides. In particular embodiments, the homology-directed repair template polynucleotide is complementary to a portion of a polynucleotide including the target sequence. When optimally aligned, a homology-directed repair template polynucleotide overlaps with one or more nucleotides of a target sequence (e.g., 1, 5, 10, 15, 20, 25, 30, 35, 40, 45, 50, 60, 70, 80, 90, 100 or more nucleotides).

In particular embodiments, the homology-directed repair template can include sufficient homology to a genomic sequence at the cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. 25, 50, 100, or 200 nucleotides, or more than 200 nucleotides of sequence homology between a homology-directed repair template and a targeted genomic sequence (or any integral value between 10 and 200 nucleotides, or more) can support HDR. Homology arms or flanking sequences are generally identical to the genomic sequence, for example, to the genomic region in which the double stranded break (DSB) occurs. However, absolute identity is not required.

In particular embodiments, the donor template includes a heterologous therapeutic nucleic acid sequence flanked by two regions of homology, such that homology-directed repair between the target DNA region and the two flanking sequences results in insertion of the heterologous therapeutic nucleic acid sequence at the target region.

In some examples, homology arms or flanking sequences of homology-directed repair templates are asymmetrical.

As indicated, in particular embodiments, donor templates include a therapeutic nucleic acid sequence. Therapeutic nucleic acid sequences can include a corrected gene sequence; a complete gene sequence and/or one or more regulatory elements associated with expression of the gene. A corrected gene sequence can be a portion of a gene requiring correction or can provide a complete replacement copy of a gene. A corrected gene sequence can provide a complete copy of a gene, without necessarily replacing an existing defective gene. One of ordinary skill in the art will recognize that removal of a defective gene when providing a corrected copy may or may not be required. When inserting a gene within a genetic safe harbor, a therapeutic nucleic acid sequence should include a coding region and all regulatory elements required for its expression.

Examples of therapeutic genes and gene products include skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C90RF72, a2p1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, Fancl, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.

In particular embodiments, a therapeutic gene includes a coding sequence for a therapeutic expression product (e.g., protein, RNA) and all associated regulatory elements (e.g., promoters, etc.) to result in expression of the gene product.

In particular embodiments, a therapeutic nucleic acid sequence (e.g., a gene) can be selected for incorporation into a GSH to provide for in vivo selection of the genetically modified cell. For example, in vivo selection using a cell-growth switch allows a minor population of genetically modified cells to be inducibly amplified. A strategy to achieve in vivo selection has been to employ drug selection while coexpressing a transgene that conveys chemoresistance, such as 06-methylguanine-DNA-methyltransferase (MGMT). An alternate approach is to confer an enhanced proliferative potential upon gene-modified HSC through the delivery of the homeobox transcription factor HOXB4. In particular embodiments, a suicide gene can be incorporated into the genetically modified cell so that such population of cells can be eliminated, for example, by administration of a drug that activities the suicide gene. See, for example, Cancer Gene Ther. 2012 August; 19(8):523-9; PLoS One. 2013; 8(3):e59594. and Molecular Therapy—Oncolytics (2016) 3, 16011.

Particular embodiments include contacting a blood cell with a gene editing system capable of inserting a donor template at a target blood cell GSH. In particular embodiments, the gene editing system includes crRNA capable of hybridizing to a target sequence within the GSH, and a nucleic acid encoding a nuclease enzyme such as Cpf1 or Cas9. In particular embodiments, Cas9 or Cpf1 coding sequences can include SEQ ID NOs: 215-227. In particular embodiments, Cas9 or Cpf1 amino acid sequences can include SEQ ID NOs: 228-241.

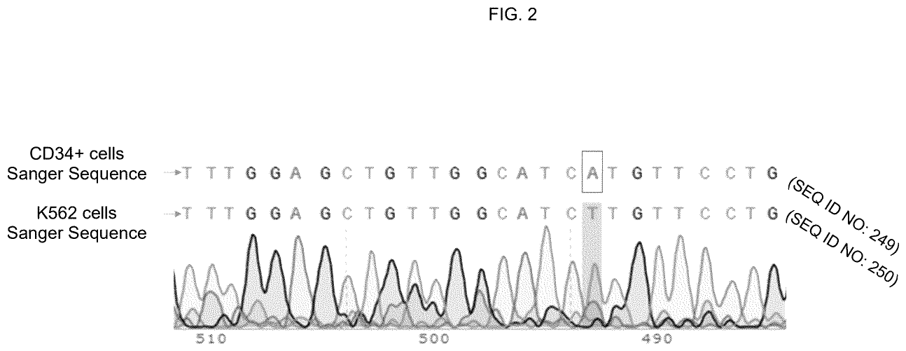

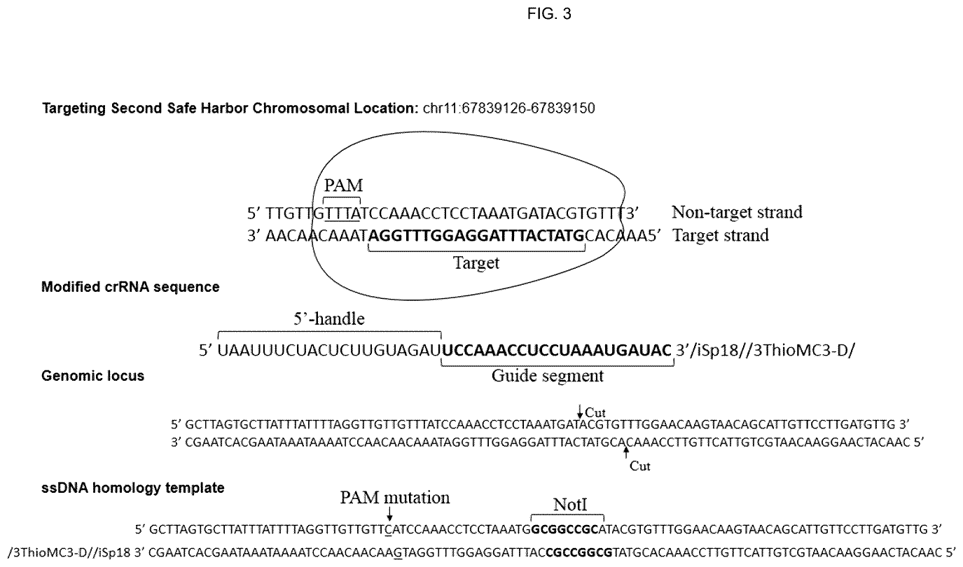

In a particular exemplary embodiment, a Cpf1/crRNA gene editing system was designed to target the chr11:67812349-67812375 genomic safe harbor (GSH) location (FIG. 1). Sanger sequencing results in K562 for this location spotted a A>T mutation in 15 bp after the PAM site (TTTG; FIG. 2) which resulted in lower cutting efficiency in this cell line compared to CD34+ cells. Also it has been shown that the cutting efficiency for the TTTA PAM site is higher than TTTG or TTTC. Therefore, another GSH location was identified in chromosome 11 (chr11: 67839126-67839150) with a TTTA PAM site which can increase cutting efficiency (FIG. 3). In addition, a SNP screen in this location did not identify any common SNP. Homology-directed repair templates (HT) were also designed. Synthesized HTs were 100 bp ssDNA bearing NotI restriction site for the assessment of HDR (FIGS. 1 and 3).

(III) Nanoparticles. As indicated previously, delivery methods of gene editing systems that do not rely on electroporation or viral vectors are needed. In addition to providing GSH and associated targeting gene editing components, the current disclosure also provides engineered nanoparticles that allow delivery of the gene editing components. The nanoparticles are engineered to include all components for targeted gene editing, for example, within a GSH. When a therapeutic use need only de-activate a problematic gene, the nanoparticles need only be associated with a targeting element and a cutting element (although other components may be included as necessary or helpful for a particular purpose). When a therapeutic use adds or corrects a gene, the nanoparticles are associated with a targeting element, a cutting element, and a donor template.

Particular embodiments utilize colloidal metal nanoparticles. A colloidal metal includes any water-insoluble metal particle or metallic compound dispersed in liquid water. A colloid metal can be a suspension of metal particles in aqueous solution. Any metal that can be made in colloidal form can be used, including gold, silver, copper, nickel, aluminum, zinc, calcium, platinum, palladium, and iron. In particular embodiments, gold nanoparticles are used, e.g., prepared from HAuCl₄. In particular embodiments, the nanoparticles are non-gold nanoparticles that are coated with gold to make gold-coated nanoparticles.

Methods for making colloidal metal nanoparticles, including gold colloidal nanoparticles from HAuCl₄, are known to those having ordinary skill in the art. For example, the methods described herein as well as those described elsewhere (e.g., US 2001/005581; 2003/0118657; and 2003/0053983) can be used to make nanoparticles.

In particular exemplary embodiments, AuNPs were synthesized in three different size ranges (15, 50, 100 nm) by an optimized Turkevich and seeding-growth methods (Shahbazi, et al., Nanomedicine (Lond), 2017. 12(16): p. 1961-1973; Shahbazi, et al., Nanotechnology, 2017. 28(2): p. 025103; Turkevich, et al. Discussions of the Faraday Society, 1951. 11(0): p. 55-75; Perrault & Chan, Journal of the American Chemical Society, 2009. 131(47): p. 17042-17043). In the first step, seed AuNPs of 15 nm were synthesized by bringing 100 mL of 0.25 mM gold (Ill) chloride trihydrate solution to the boiling point and adding 1 mL of 3.33% trisodium citrate dehydrate solution. Synthesis of nanoparticles was carried out in high stirring speeds over 10 min. Prepared nanoparticles were cooled down to 4° C. and used in the following growth step.

In order to prepare AuNPs in 50 nm and 100 nm size ranges, two different 100 mL of 0.25 mM gold (Ill) chloride trihydrate solutions were prepared and in mild stirring conditions 2440 μL and 304 μL of seed AuNPs were added separately to synthesize 50 nm and 100 nm AuNPs, respectively. To these solutions was added 1 mL of 15 mM trisodium citrate dehydrate solution and the mixture was brought to the highest stirring speed. Then, 1 mL of 25 mM hydroquinone solution was added and synthesis was continued over 30 min for 50 nm AuNPs and 5 h for 100 nm AuNPs. Finally, synthesized nanoparticles were purified by centrifuging at 5000×g and dispersing in ultra-pure water.

While AuNPs are particularly described, nanoparticles encompassed in the present disclosure may be provided in different forms, e.g., as solid nanoparticles (e.g., metal such as silver, gold, iron, titanium), non-metal, lipid-based solids, polymers, suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Semi-solid and soft nanoparticles have been manufactured, and are within the scope of the present disclosure. A nanoparticle of a semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

A nanoparticle can include any suitable material, e.g., a biocompatible material. The biocompatible material can be a polymer. Suitable nanoparticle polymers include polystyrene, silicone rubber, polycarbonate, polyurethanes, polypropylenes, polymethylmethacrylate, polyvinyl chloride, polyesters, polyethers, and polyethylene. Examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), poly anhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyethylenimine (PEI), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), trimethylene carbonate, and polyvinylpyrrolidone.

In particular embodiments, the nanoparticle is a lipid nanoparticle. A lipid nanoparticle can include one or more lipids, and one or more of the polymers listed above.

Lipidoid compounds are also particularly useful in the administration of gene editing system components. In particular embodiments, aminoalcohol lipidoid compounds are combined with gene editing system components to be delivered to a cell or a subject to form microparticles, nanoparticles, liposomes, or micelles. The gene editing system components to be delivered by the particles, liposomes, or micelles may be a polynucleotide, protein, peptide, or small molecule. Aminoalcohol lipidoid compounds may be combined with other aminoalcohol lipidoid compounds, polymers (synthetic or natural), surfactants, cholesterol, carbohydrates, proteins, lipids, etc. to form particles.

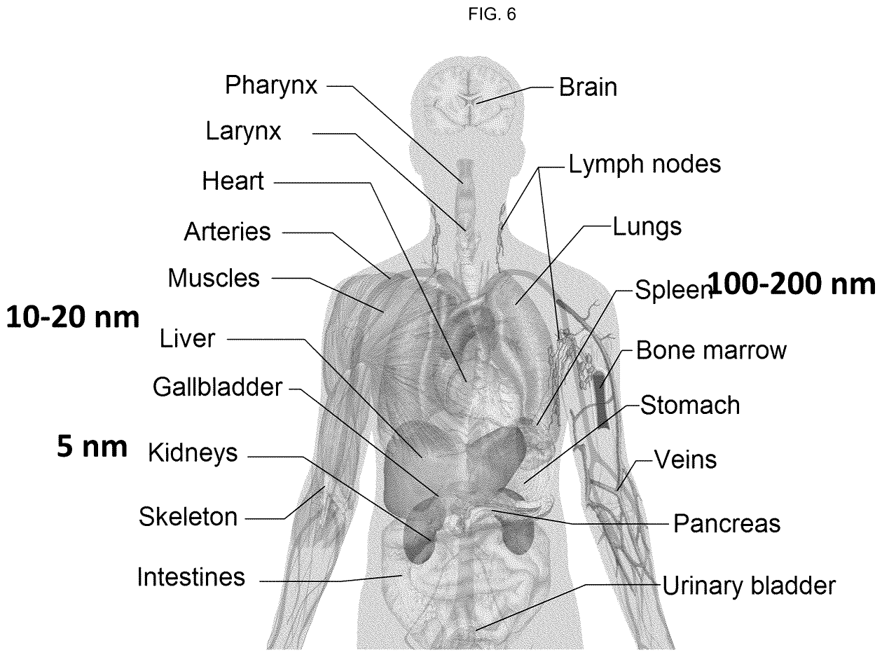

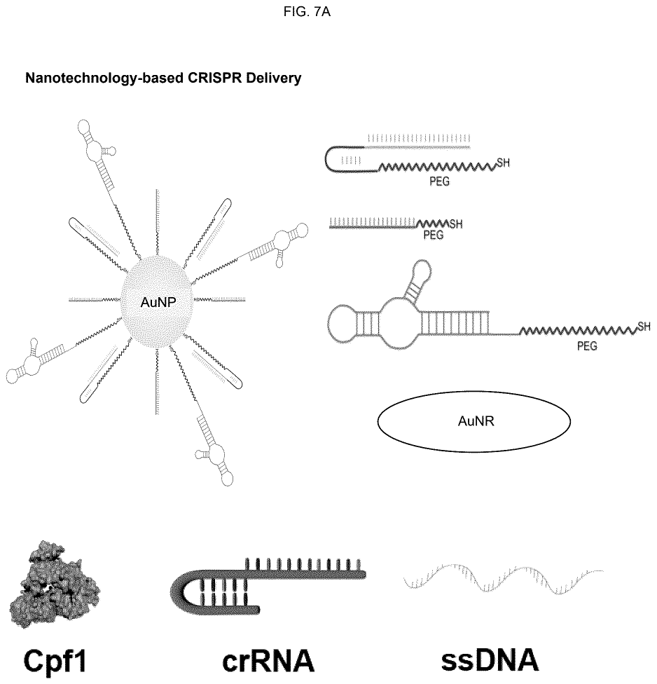

As depicted in FIG. 6, the size of an AuNP can be selected to affect biodistribution within the human body. Nanoparticles suitable for use in the present disclosure can be any shape and can range in size from 5 nm-1000 nm in size, e.g., from 5 nm-10 nm, 5-50 nm, 5 nm-75 nm, 5 nm-40 nm, 10 nm-30, or 20 nm-30 nm. Nanoparticles can also have a size in the range of from 10 nm-15 nm, 15 nm-20 nm, 20 nm-25 nm, 25 nm-30 nm, 30 nm-35 nm, 35 nm-40 nm, 40 nm-45 nm, or 45 nm-50 nm, 50 nm-55 nm, 55 nm-60 nm, 60 nm-65 nm, 65 nm-70 nm, 70 nm-75 nm, 75 nm-80 nm, 80 nm-85 nm, 85 nm-90 nm, 90 nm-95 nm, 95 nm-100 nm, 100 nm-105 nm, 105 nm-110 nm, 110 nm-115 nm, 115 nm-120 nm, 120 nm-125 nm, 125 nm-130 nm, 130 nm-135 nm, 135 nm-140 nm, 140 nm-145 nm, 145 nm-150 nm, 100 nm-500 nm, 100 nm-150 nm, 150 nm-200 nm, 200 nm-250 nm, 250 nm-300 nm, 300 nm-350 nm, 350 nm-400 nm, 400 nm-450 nm, or 450 nm-500 nm. In particular embodiments, nanoparticles greater than 550 nm are excluded. This is because particles or aggregated particles of >600 nm are not amenable to cellular uptake.

Particular embodiments can also include nanoparticles associated with targeting molecules. Targeting molecules can be used to target the nanoparticle to a specific cell so that activity of the gene editing system can be spatially or temporally controlled. For example, the activity and destination of the gene editing system can be controlled by a targeting molecule that has binding affinity for a cell surface protein or other localized cellular component.

In particular embodiments, targeting molecules include antibodies or binding domains thereof that result in selective delivery of nanoparticles to selected cell types. In particular embodiments, selective delivery is exclusive to a selected cell population. In particular embodiments, at least 65%, 70%, 75%, 80%, 85%, 90%, 95% or 99% of administered nanoparticles are delivered to a selected cell population.

In particular embodiments, binding domains include cell marker ligands, receptor ligands, antibodies, peptides, peptide aptamers, nucleic acids, nucleic acid aptamers, spiegelmers or combinations thereof. Within the context of selected cell targeting ligands, binding domains include any substance that binds to another substance to form a complex capable of supporting selective delivery.

As indicated, “antibodies” are one example of binding domains and include whole antibodies or binding fragments of an antibody, e.g., Fv, Fab, Fab′, F(ab′)₂, Fc, and single chain Fv fragments (scFvs) or any biologically effective fragments of an immunoglobulin that bind specifically to a motif expressed by a selected cell type. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Antibodies from human origin or humanized antibodies have lowered or no immunogenicity in humans and have a lower number of non-immunogenic epitopes compared to non-human antibodies. Antibodies and their fragments will generally be selected to have a reduced level of antigenicity in human subjects.

In particular embodiments, HSCs are targeted for selective delivery of nanoparticles. In particular embodiments, HSCs are targeted for selective delivery with binding domains that selectively bind CD34 and CD90. In particular embodiments, HSC can be targeted for selective delivery with one or more binding domains that selectively bind known antigens expressed on the surface of HSCs and HSPCs: CD34, CD46, CD90, CD133, Sca-1 and/or CD117.

Mature T cells can be targeted for selective delivery with binding domains that selectively bind CD3. Activated T-cells can be targeted for selective delivery with binding domains that selectively bind 4-11BE (CD137), CD69, and/or CD25. T helper cells can be targeted for selective delivery with binding domains that selectively bind CD4. Cytotoxic T-cells can be targeted for selective delivery with binding domains that selectively bind CD8. “Central memory” T-cells (or “TCM”) can be targeted for selective delivery with binding domains that selectively bind CD62L, CCR7, CD25, CD127, CD45RO, and/or CD95. “Effector memory” T-cell (or “TEM”) can be targeted for selective delivery with binding domains that selectively bind granzyme B and/or perforin. Regulatory T cells (“TREG”) can be targeted for selective delivery with binding domains that selectively bind CD25, CTLA-4, GITR, GARP and/or LAP. “Naive” T-cells can be targeted for selective delivery with binding domains that selectively bind CD62L, CCR7, CD28, CD127 and/or CD45RA.

Natural killer cells (also known as NK cells, K cells, and killer cells) can be targeted for selective delivery with binding domains that selectively bind CD8, CD16 and/or CD56.

Macrophages (and their precursors, monocytes) can be targeted for selective delivery with binding domains that selectively bind CD11b, F4/80; CD68; CD11c; IL-4Rα; and/or CD163.

Immature dendritic cells (i.e., pre-activation) can be targeted for selective delivery with binding domains that selectively bind: CD1a, CD1b, CD1c, CD1d, CD21, CD35, CD39, CD40, CD86, CD101, CD148, CD209, and/or DEC-205.

B cells can be targeted for selective delivery with binding domains that selectively bind CD5, CD19, CD20, CD21, CD22, CD35, CD40, CD52, and/or CD80.

Lymphocyte function-associated antigen 1 (LFA-1) is expressed by all T-cells, B-cells, and monocytes/macrophages. Accordingly, selected cell targeting ligands can bind LFA-1 to achieve selective delivery of nanoparticles to T-cells, B-cells, and monocytes/macrophages.

In particular embodiments, a targeting molecule can be responsive to, i.e. activated or inactivated by, an effector on or in the cell. In particular embodiments, other components within a sequence can include regulatory nucleotides such as a promoter element, a small interfering or hairpin RNA or a microRNA to control expression of another gene in the same cell, or a DNA barcode for cellular tracking.

Aptamers may be designed to facilitate selective delivery, including delivery across the cellular membrane, to intracellular compartments, or into the nucleus. Such a structure can include, either in addition to the one or more aptamer(s) or without such one or more aptamer(s), moiety(ies) so as to render the guide deliverable, inducible or responsive to (for example activatable or inactivatable by) a selected effector, for example responds to normal or pathological physiological conditions, including without limitation pH, hypoxia, O₂ concentration, temperature, protein concentration, enzymatic concentration, lipid structure, light exposure, mechanical disruption (e.g., ultrasound waves), magnetic fields, electric fields, or electromagnetic radiation. Methods of making aptamers and conjugating such aptamers to the surface of a nanoparticle are known in the art, see for example Huang et al. Anal. Chem., 2008, 80 (3), pp 567-572.

In particular embodiments, an RNA aptamer sequence has binding affinity for an aptamer ligand on or in the cell. In particular embodiments, the aptamer ligand is on the cell, for example so that it is at least partially available on the extra-cellular face or side of the cell membrane. For example, the aptamer ligand may be a cell-surface protein. The aptamer ligand may therefore be one part of a fusion protein, one other part of the fusion protein having a membrane anchor or membrane-spanning domain. In particular embodiments, the aptamer ligand is in the cell. For example, the aptamer ligand may be internalized within a cell, i.e. within (beyond) the cell membrane, for example in the cytoplasm, within an organelle (including mitochondria), within an endosome, or in the nucleus. In particular embodiments, an aptamer can include a donor template sequence, which can include an HDR template and a therapeutic nucleic acid sequence.

(IV) Conjugation of Active Components to Nanoparticles. As indicated, a variety of active components can be conjugated to the nanoparticles disclosed herein for targeted gene editing. For example, nucleic acids that are gene editing system components can be conjugated directly or indirectly, and covalently or noncovalently, to the surface of the nanoparticle. For example, a nucleic acid may be covalently bonded at one end of the nucleic acid to the surface of the nanoparticle.

Nucleic acids conjugated to the nanoparticle can have a length of from 10 nucleotides (nt)-1000 nt, e.g., 1 nt-25 nt, 25 nt-50 nt, 50 nt-100 nt, 100 nt-250 nt, 250 nt-500 nt, 500 nt-1000 nt or greater than 1000 nt. In particular embodiments, nucleic acids modified by conjugation to a linker do not exceed 50 nt or 40 nt in length.

When conjugated indirectly through, for example, an intervening linker, any type of molecule can be used as a linker. For example, a linker can be an aliphatic chain including at least two carbon atoms (e.g., 3, 4, 5, 6, 7, 8, 9, 10, 11, 12, 13, 14, or more carbon atoms), and can be substituted with one or more functional groups including a ketone, ether, ester, amide, alcohol, amine, urea, thiourea, sulfoxide, sulfone, sulfonamide, and/or disulfide.

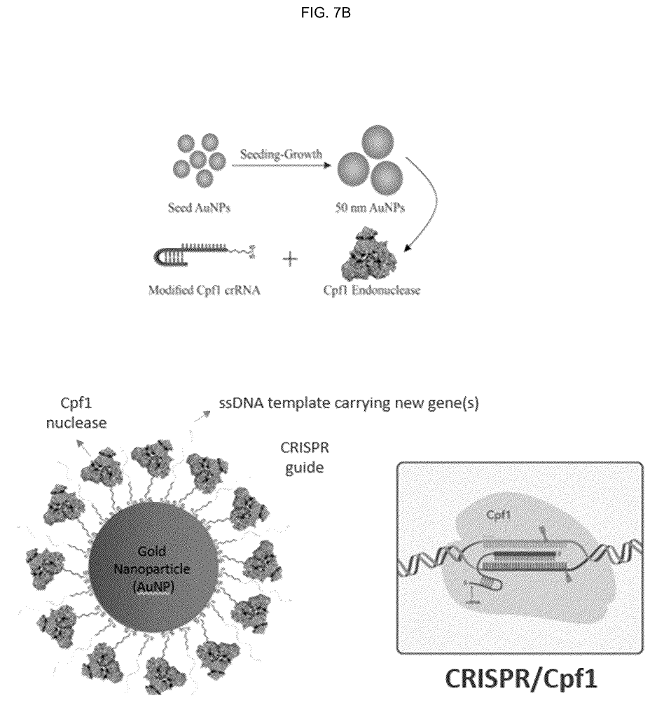

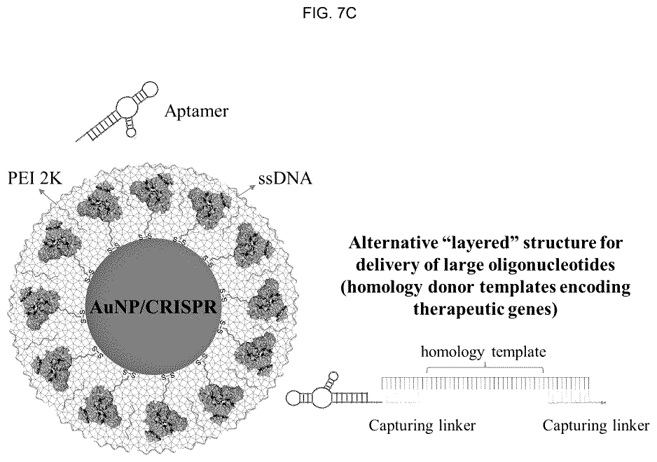

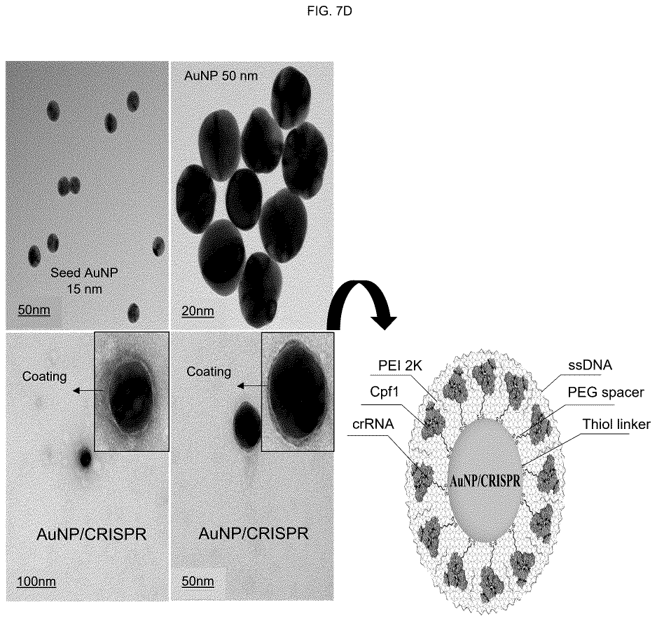

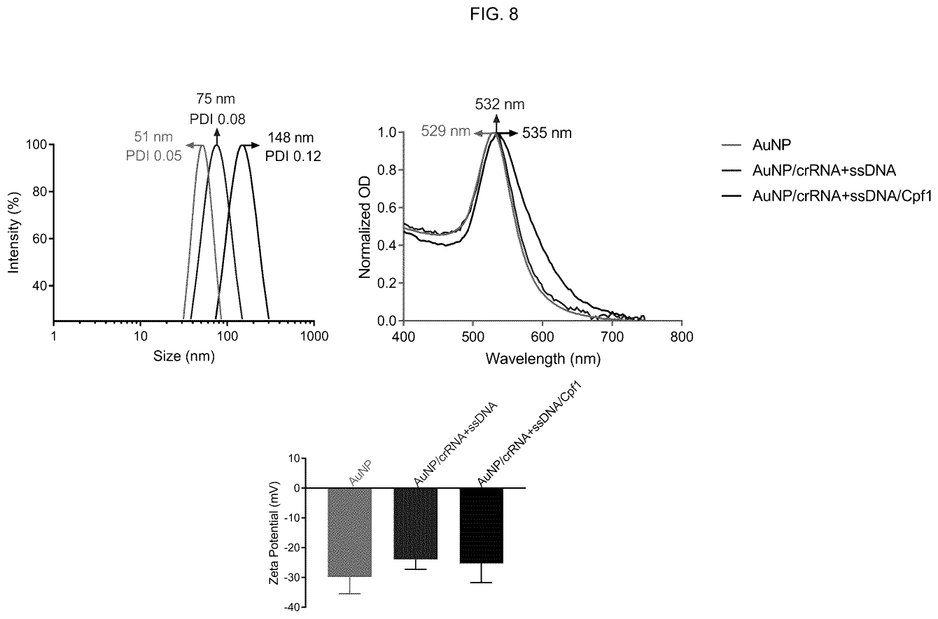

In particular embodiments the linker includes a disulfide at the free end (e.g. the end not conjugated to the guide RNA) that couples the nanoparticle surface. In particular embodiments, the disulfide is a C₂-C₁₀ disulfide, that is it can be an aliphatic chain terminating in a disulfide that includes 2, 3, 4, 5, 6, 7, 8, 9, or 10 carbon atoms, although it is envisioned that longer aliphatic chains can be used. In particular embodiments, the disulfide is a 3 carbon disulfide (C3 S—S). Linkers can have either sulfhydryl groups (SH) or disulfide groups (S—S) or a different number of sulfur atoms. In particular embodiments, a thiol modification can be introduced without using a linker. In particular embodiments, a nuclease enzyme is delivered as a protein pre-conjugated with its guide RNA (a ribonucleoprotein (RNP) complex). In this formulation, the guide RNA molecule is bound to the nanoparticle and the nuclease enzyme, by default, can be also bound (see, for example, FIGS. 7B and 7D).

One advance disclosed herein is the ability to modify CRISPR components for linkage to a nanoparticle. This is because most of the modifications in CRISPR components can compromise cutting efficiency. For example, Li et al. (Engineering CRISPR-Cpf1 crRNAs and mRNAs to maximize genome editing efficiency. 2017. 1: p. 0066) indicated that the 5′ end of Cpf1 crRNA is not safe for any modification because such modifications result in the abrogation of the crRNA binding to Cpf1 nuclease. Disclosed herein is a modification to the 3′ end of crRNA that does not compromise cutting efficiency. In particular embodiments, in the first step of conjugation to a nanoparticle the 3′ end of the crRNA is modified with an 18-atom hexa-ethyleneglycol spacer (18 spacer) and 3 carbon disulfide (C3 S—S) to attach the crRNA to the surface of AuNPs.

Based on the foregoing, in particular embodiments, for example when the nanoparticle includes gold, a linker can be any thiol-containing molecule. Reaction of a thiol group with Au results in a covalent sulfide (—S—) bond. AuNPs have high affinity to thiol (—SH) and dithiol (S—S) groups and semi-covalent bonds occur between the surface of AuNP and sulfur groups (Hakkinen, Nat Chem, 2012. 4(6): p. 443-455). In particular embodiments, thiol groups can be added to nucleic acids to facilitate attachment to the surface of AuNPs. This approach can improve nucleic acid uptake and stability (see, e.g., Mirkin, et al., A Nature, 1996. 382(6592): p. 607-609).

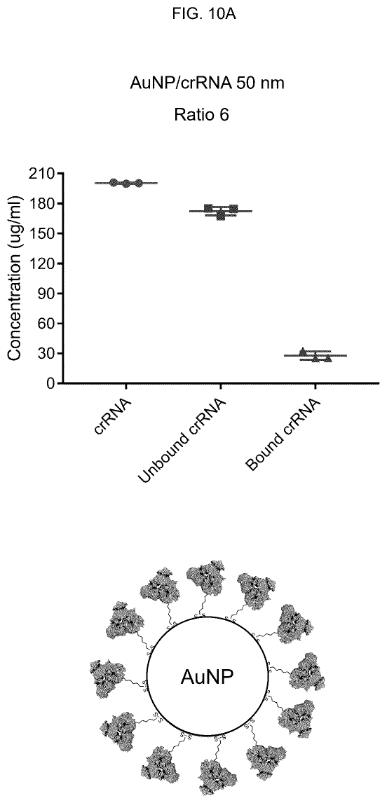

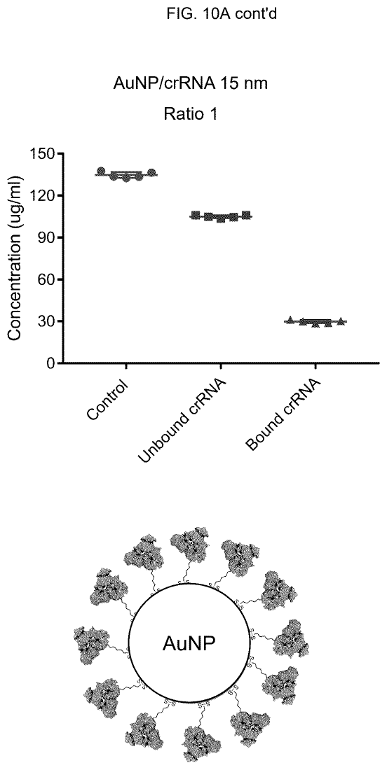

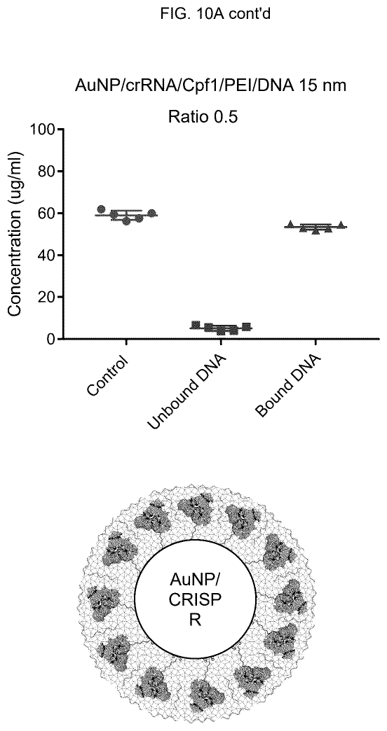

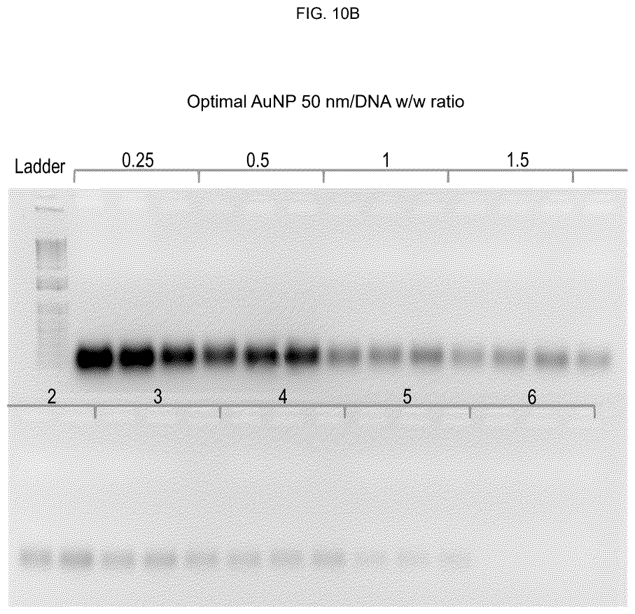

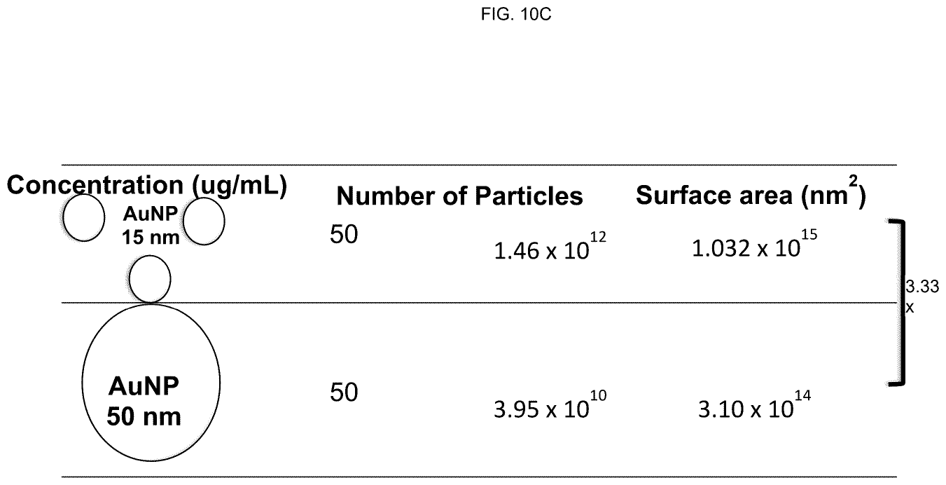

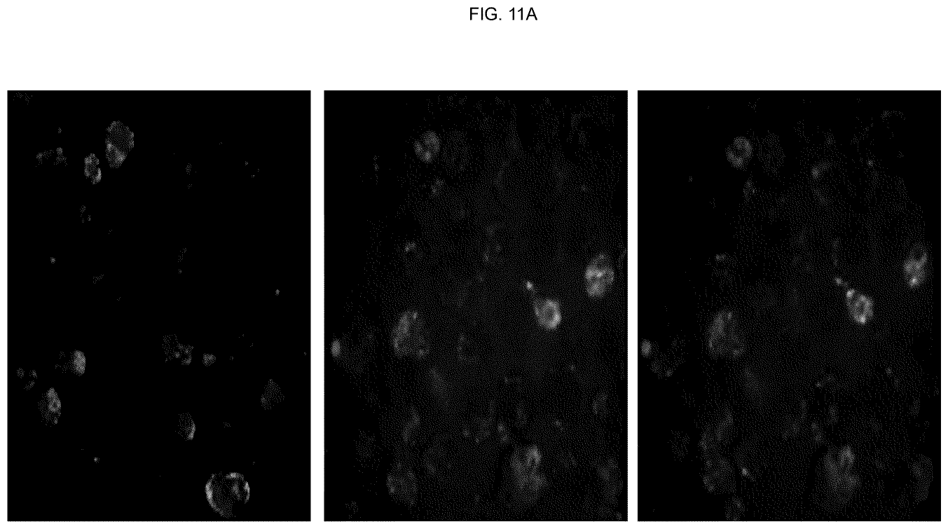

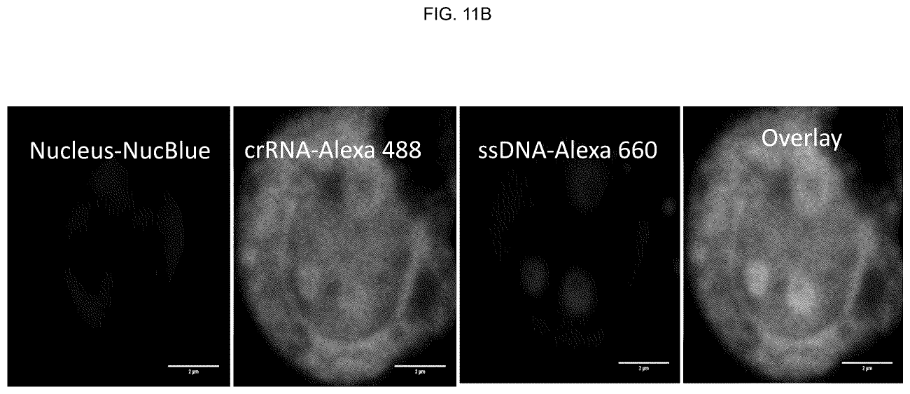

Using an optimized two step method of seeding-growth, highly monodisperse AuNPs were synthesized in 3 different size ranges (15 nm, 50 nm, 100 nm) and conjugated with Cpf1 crRNA and endonuclease (FIGS. 7B, 7D, 10C). Because of the strong electrostatic repulsion between the negatively charged surface and negatively charged crRNA it is difficult to attach the crRNA to the surface of AuNPs without, for example, the thiol modification. In particular embodiments, in the second step, after purification of the crRNA conjugated AuNPs, Cpf1 endonuclease is added and incubated with crRNA conjugated AuNPs to facilitate its binding to the 5′ handle of the crRNA (Dong, et al., Nature, 2016. 532(7600): p. 522-526). The compact structure of the designed nanoformulation containing both crRNA and Cpf1 endonuclease results in a conformation which increases the stability against degrading agents and facilitates the uptake of the AuNP/CRISPR nanoformulation by cells owing to an overall neutral charge (i.e., zeta potential). While special relevance was given to optimizing the disclosed nanoformulation for CRISPR/Cpf1, the same concept may be applied to other CRISPR classes. Also, along with the crRNA and Cpf1 endonuclease, 18 spacer thiol modified single stranded DNA (ssDNA) can be attached to the surface of AuNPs to obtain a novel nanoformulation with the aim of being used in homology directed repair (HDR).

In particular embodiments, a spacer-thiol linker can be added to either of the Cpf1 or Cas9 proteins themselves or engineered variants of the foregoing (e.g., as described below), by addition of a cysteine residue on either the N- or C-terminus. The nuclease protein can then be added as a first layer on the gold nanoparticle surface. This spacer-thiol linker can increase the stability of the protein and increase cutting efficiency. In particular embodiments, an RNA complex is formed between crRNA and nuclease and then attached to the surface of gold nanoparticles through a spacer-thiol linker.

As indicated previously, adding gene-editing components of a bacterial origin as a first loading step can provide beneficial shielding of these components following administration to a subject with pre-existing immunity to the component. The shielding can be due to other gene-editing components (e.g., donor templates) and need not rely on a protective polymer shell. In particular embodiments, a polymer shell is excluded. In particular embodiments, the shielding may permit serial in vivo administration.

In particular embodiments, crRNAs can be added to AuNPs in different AuNP/crRNA w/w ratios (0.25, 0.5, 1, 1.5, 2, 3, 4, 5, 6) and mixed. Citrate buffer with the pH of 3 can be added to the mixture in 10 mM concentration to screen the negative repulsion between negatively charged crRNA and AuNP. After stirring for 5 min, nanoparticles can be centrifuged down and the unbound crRNA can be visualized by agarose gel electrophoresis. After determining the optimal conjugation concentration, 1 μL of 63 μM Cpf1 nuclease can be added to AuNP/crRNA solution and incubated for 20 min.

Importantly, the use of a citrate buffer provides significant advantages in manufacturing. Previous methods have relied on the use of NaCl to screen the negatively-charged nanoparticle surface and reduce repulsion of similarly negatively-charged DNA. However, NaCl can cause irreversible aggregation of gold nanoparticles, so it must be added gradually over time with incremental changes in concentration. Generally, NaCl must be added over a 48 hour time period to avoid aggregation. When citrate buffer is used with a pH of 3, this binding can happen with higher efficiency in less than 3 minutes. Zhang, et al. (2012). Journal of the American Chemical Society 134(17): 7266-7269 reducing the cost of goods and time in the GMP manufacturing facility.

Size and morphology of prepared AuNP/CRISPR nanoformulations can be characterized by imaging under transmission electron microscope (TEM). AuNPs (4 μL) can be added to copper grids and allowed to dry out overnight. Imaging is carried out at 120 kV.

CRISPR coating can be visualized by negative staining electron microscopy. AuNP/CRISPR nanoformulation can be stained with 0.7% uranyl formate and 2% uranyl acetate, respectively. Stained sample (4 μL) can be added to carbon-coated copper grid and incubated for 1 min and blotted with a piece of filter paper. After three washing cycles with 20 μl stain solution, 4 μl stain solution can be added to the grids and blotted and air dried.

Also, AuNP/CRISPR nanoformulations can be characterized by Nanodrop UV-visible spectrophotometer by analyzing the shifts in localized surface plasmon resonance (LSPR) peak of the AuNPs before and after conjugation with CRISPR components.

In particular embodiments, a nanoparticle is layered, such as during synthesis to include PEI or other positively charged polymer for increasing surface area and conjugating larger ssDNA or other molecules, such as targeting molecules and/or large donor templates (see, for example, FIG. 7C). This nanoformulation can be prepared in a layer by layer form and positively charged polymers (such as; PEI in different molecular weights and forms) can be used to coat the negatively charged surface of either gold nanoparticles or CRISPR coated gold nanoparticles to attach either gene editing components and other components (such as antibody binding domains). Layering essentially increases the surface area of the nanoparticle available for conjugating molecules such as large oligonucleotides with or without other proteins.

In particular embodiments, PEI can be added as a second layer and ssDNA can be added as a third layer. Alternatively, the conjugation steps can be changed by adding ssDNA as a second layer and PEI as a third layer. In particular embodiments, PEI, polymers, and ssDNA are not included as a first layer, as this layer can be reserved for RNP complexes coupled to linkers.

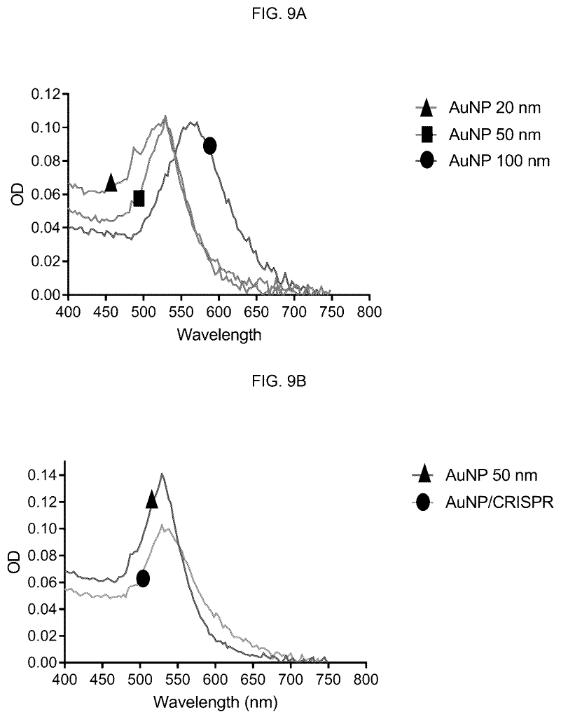

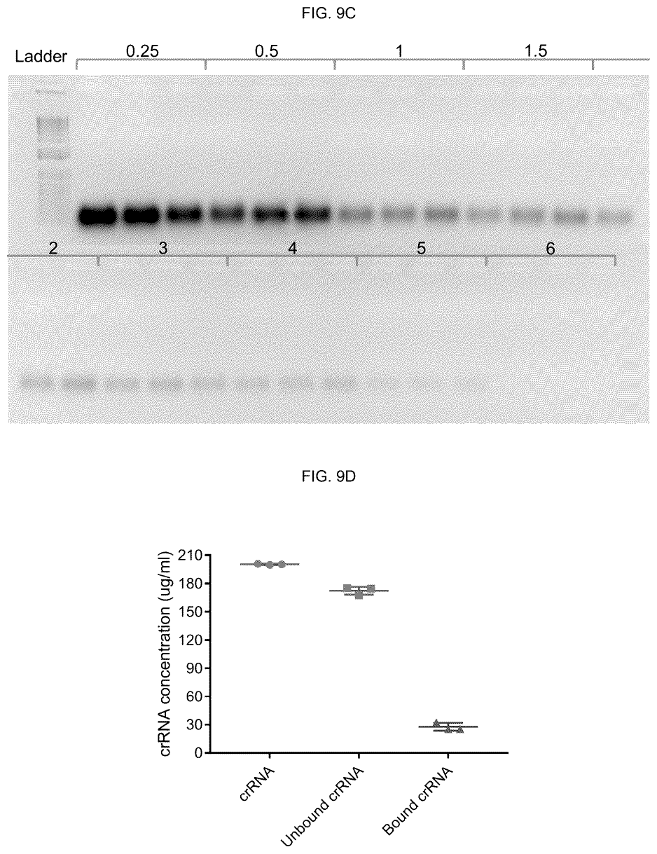

In particular embodiments, a multilayered nanoformulation of the disclosure has an average size of 25-30 nm and is highly monodisperse. Transmission electron microscope images (TEM) and localized surface plasmon resonance shifts (LSPR) of gold nanoparticles (AuNPs) showed a uniform surface coating without any aggregation (FIGS. 9A, 9B). Given the synthetic nature of the entire delivery system, all components can be assembled within a few hours, as opposed to previous approaches which required multiple days due to, for example, use of NaCl as a charge screen.

As shown in FIGS. 7D and 9A, synthesized nanoparticles were highly monodisperse and successful 4 nm coating without any aggregation was achieved which increased the size of the nanoparticles to 54 nm after coating for 50 nm AuNPs. Also, decrease in the intensity and red shifting of the localized surface plasmon (LSPR) of AuNPs showed the successful conjugation with CRISPR components without any aggregation (FIG. 9A). Also, oligonucleotide loading studies with the model 18 spacer C3 S—S modified ssDNA in different AuNP/ssDNA w/w ratios showed that the ratio of 6 is optimal to carry out the conjugation (FIG. 9C). Using this optimal loading ratio crRNA was loaded on the surface of AuNPs in 30 μg/mL concentration (FIG. 9D). These data help calculate the exact application dosage for gene editing studies.

The described approaches resulted in a highly potent, loaded, CRISPR nanoformulation capable of delivering both synthetic, non-chemically modified CRISPR Cpf1 or CRISPR Cas9 ribonucleoproteins along with a ssDNA homology template for insertion of new DNA, without the need for electroporation (FIG. 7D). In particular embodiments, the hydrodynamic size of a fully loaded AuNP is 150-190 nm, 160-185 nm, 170-180 nm or 176 nm.

(V) Gene Editing Efficiency. The optimal concentrations of crRNA, hAsCpf1 RNA and ssODN for electroporation were determined in K562 cells. The optimal concentration displays the highest viability and GFP expression. K562 cells were cultured in 24 well plates in 1×10⁵ cells/well concentration. Iscove's Modified Dulbecco's Medium (IMDM) with 10% FBS and 1% PenStrep was used to culture the cells. CD34+ cells were cultured in 24 well plates in 5×10⁵ cells/well concentration. Culture conditions for CD34+ cells were the same as K562 cells with required growth factors. AuNP/CRISPR nanoformulations were added in 25 nM concentration to the wells and editing efficiency was evaluated after 48 h incubation. Electroporation of the cells was performed with a Harvard Apparatus ECM 830 Square Wave Electroporation System using BTX Express Solution (USA). in 1 mm cuvettes in 250 V and 5 ms pulse duration. 1 mm BTX cuvettes with a 2 mm gap width were used to electroporate 1-3 million K562 cells at 250V for 5 milliseconds. Cells were resuspended in culture media and analyzed following electroporation.

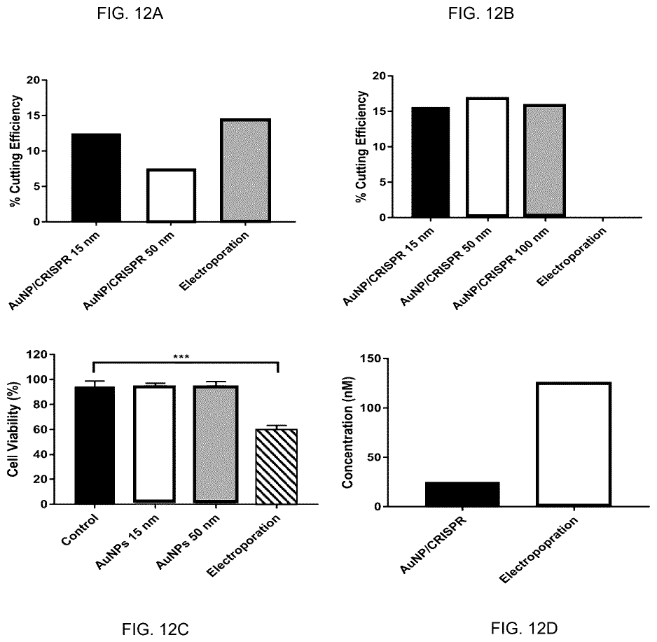

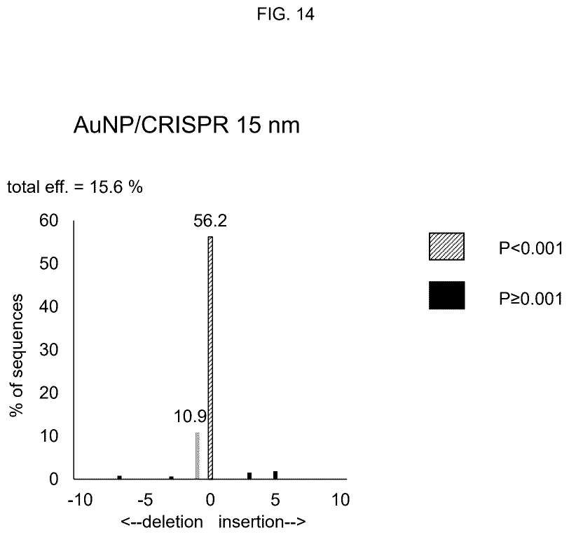

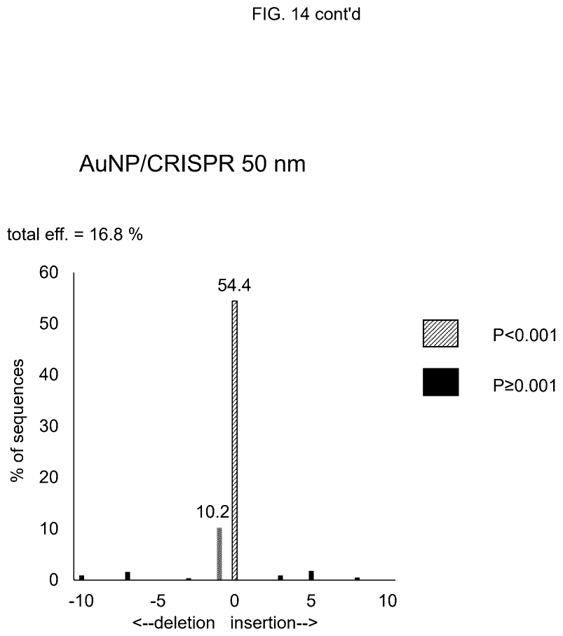

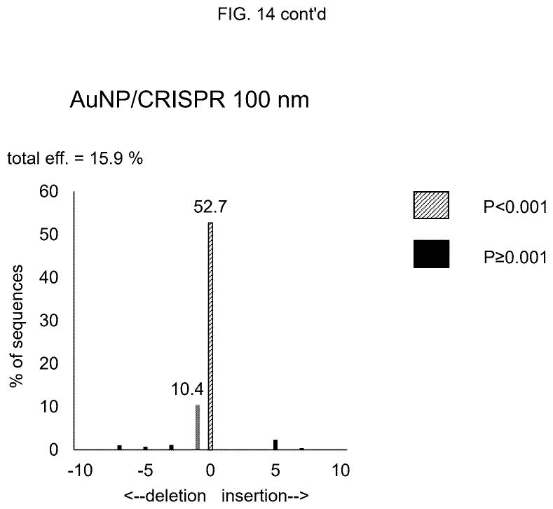

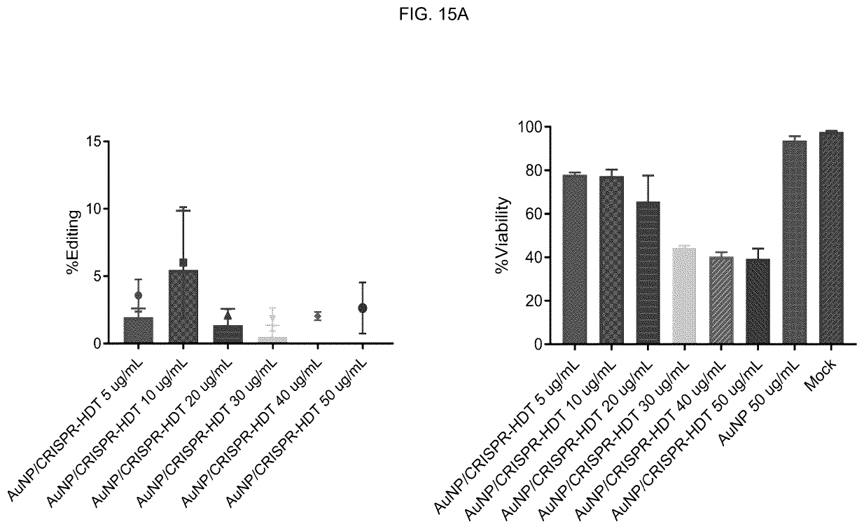

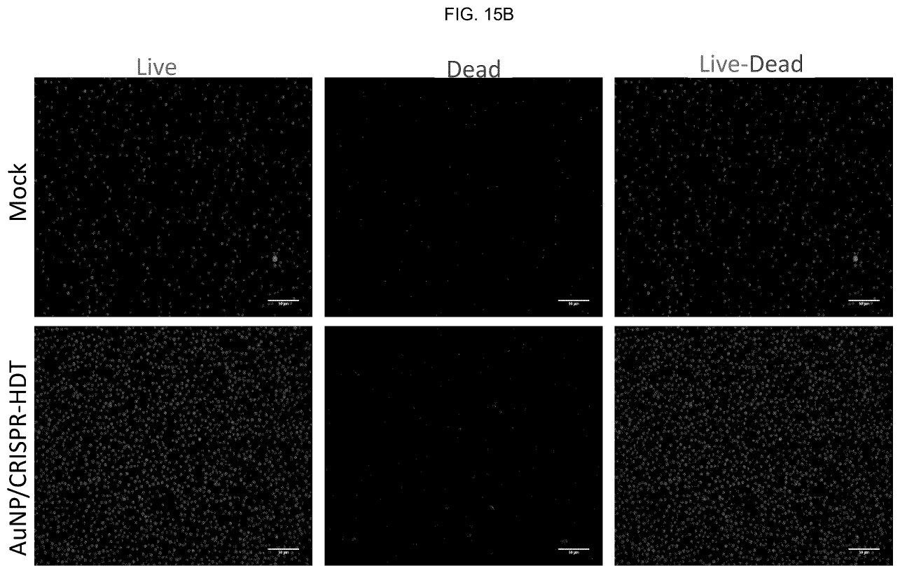

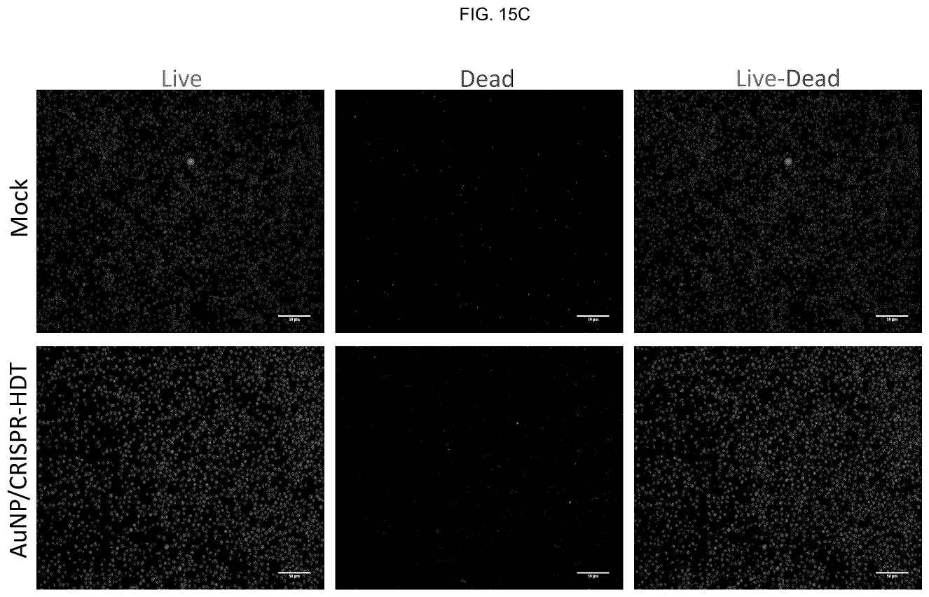

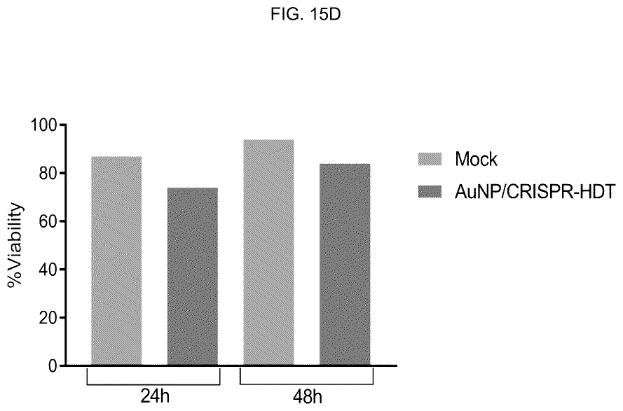

AuNP/CRISPR nanoformulations targeting the chr11:67812349-67812375 location were able to successfully cut the target site in very low crRNA and Cpf1 endonuclease concentrations (25 nM) in comparison to electroporation method in which a higher amount of crRNA and Cpf1 was used (126 nM) (FIG. 12A) to achieve the same efficiency of cutting. Cutting efficiency for this site was low due to the A>T mutation 15 bp after the PAM site (FIG. 2). In the next test, the same location was targeted in primary CD34+ cells and it was shown that AuNP/CRISPR nanoformulations were able to target the site in a very low crRNA and Cpf1 endonuclease concentrations with very good cutting efficiency without raising any toxic effects (FIGS. 12B, 12C, and 14). Unfortunately, electroporation of the primary CD34+ cells adversely affected the viability of the cells and no cutting was seen for electroporated cells. Calculated concentration for AuNP/CRISPR nanoformulation was 5 fold lower than required concentration for electroporation method (FIG. 12D). As previously mentioned by Kim et al. (Nat Biotechnol, 2016. 34(8): p. 863-8), the rate of deletions to insertions was higher with the CRISPR Cpf1 gene editing system (FIG. 14).

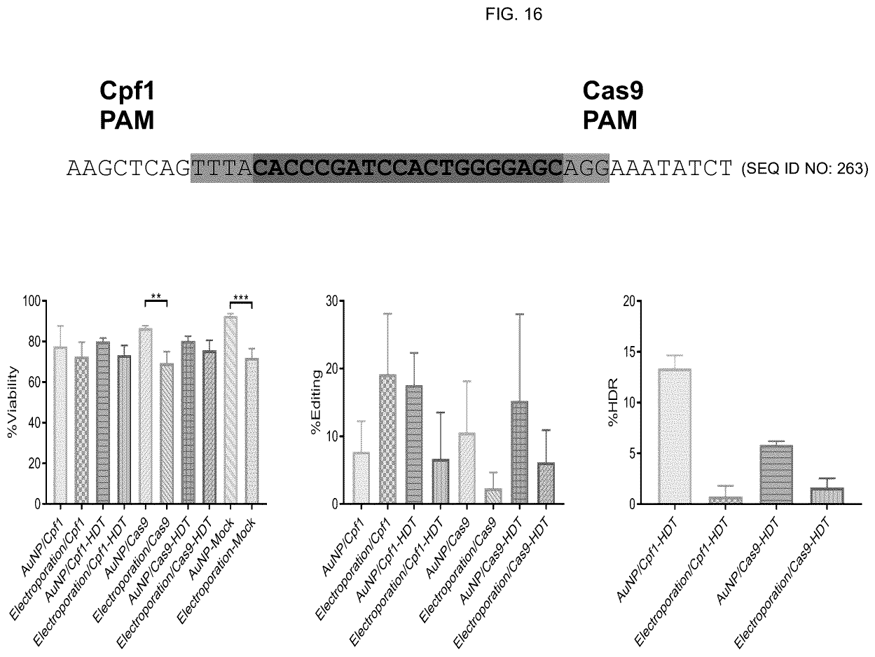

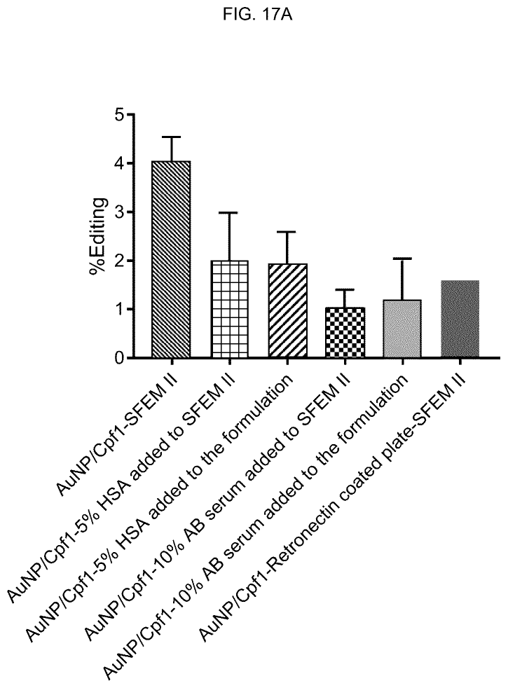

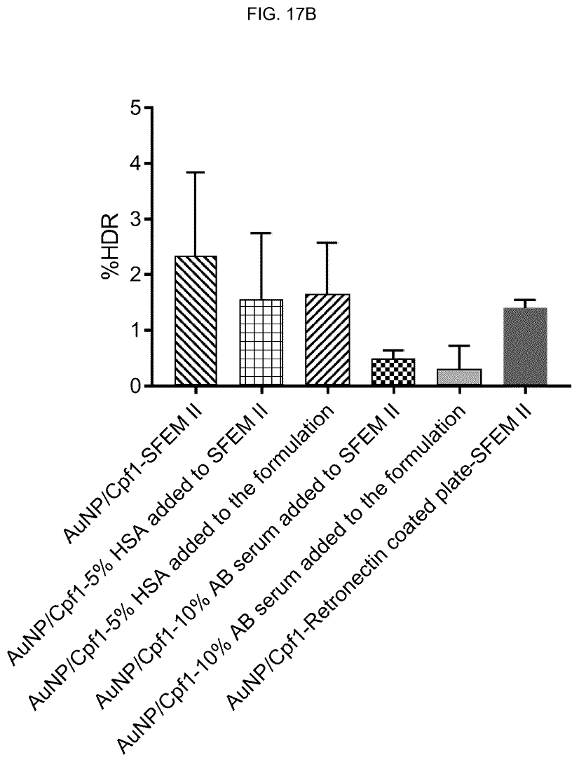

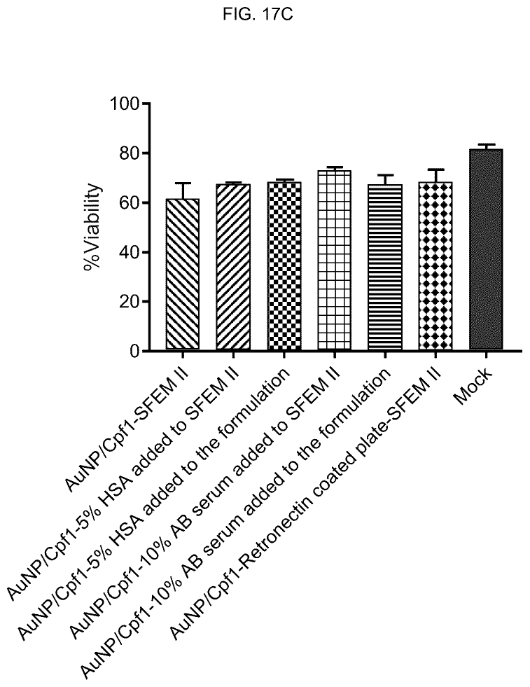

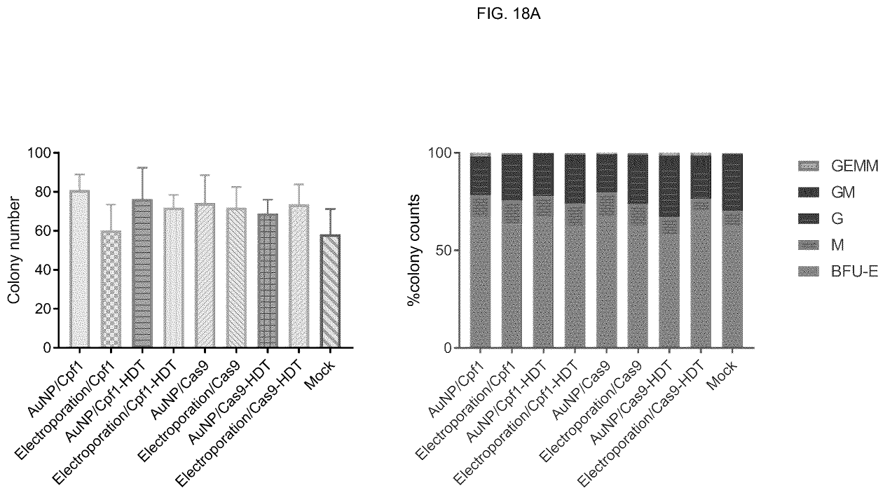

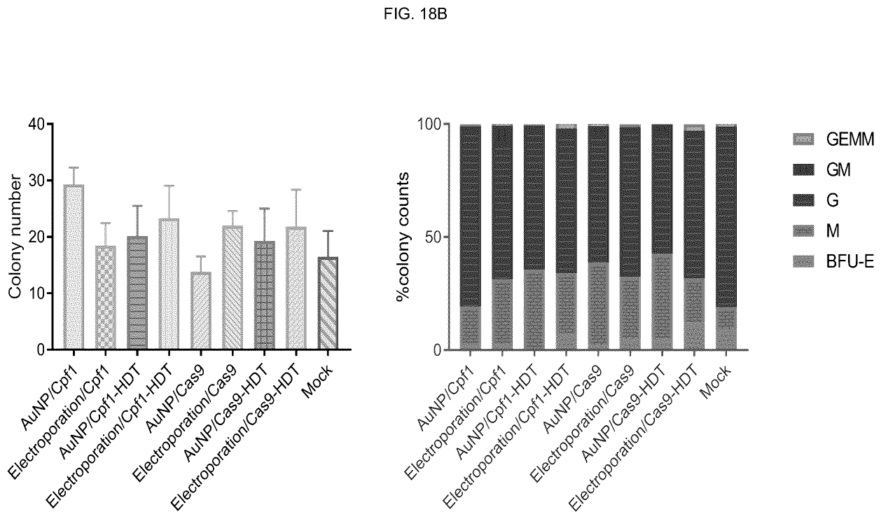

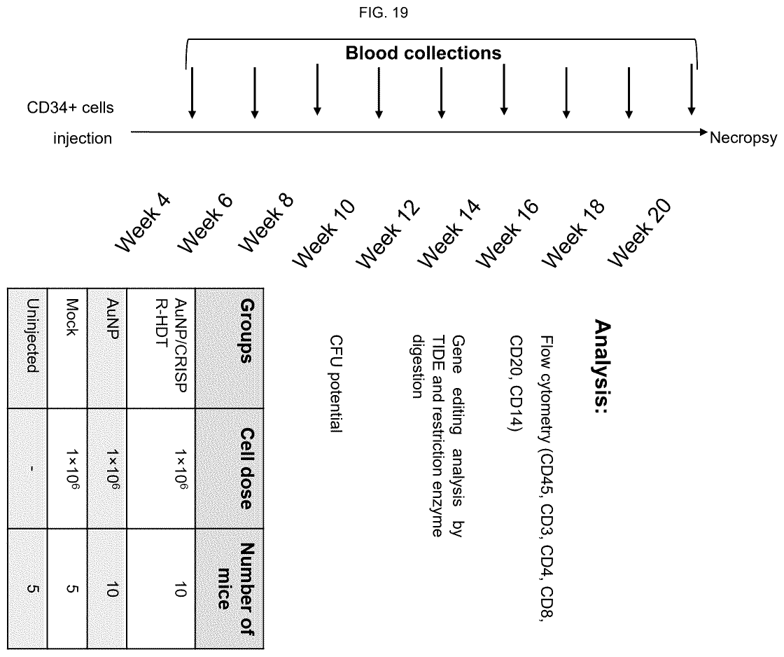

As shown in FIG. 16, AuNP-mediated gene delivery improves Cas9 performance, however, Cpf1 is better for HDR. AuNP treated cells demonstrated higher viability compared to electroporated cells. For Cas9, AuNP mediated delivery improved total editing and HDR, relative to electroporation. For Cpf1 delivered without a homology-directed repair template (HDT), electroporation resulted in higher total gene editing (indels). This suggests that electroporation itself may impact the repair pathway used or the frequency of Cpf1 cutting at the target site. Addition of HDT to the Cpf1 formulation improved total editing and resulted in the highest HDR rates. Together, these data suggest that the fully-loaded formulation of AuNP+Cpf1/crRNA+HDT results in the highest rates of HDR with minimal indel formation. This is ideal for a number of target loci for gene editing.

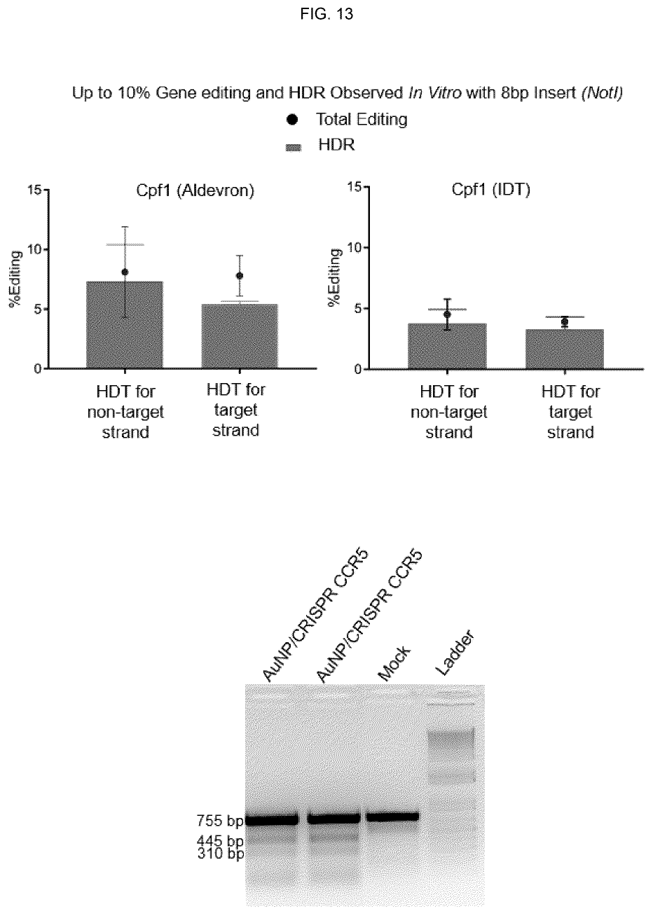

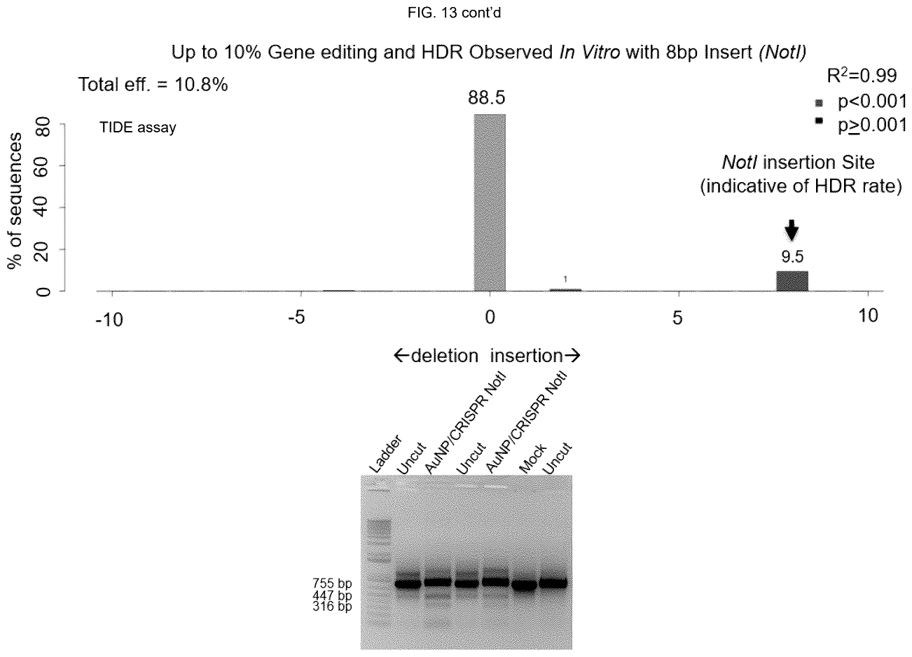

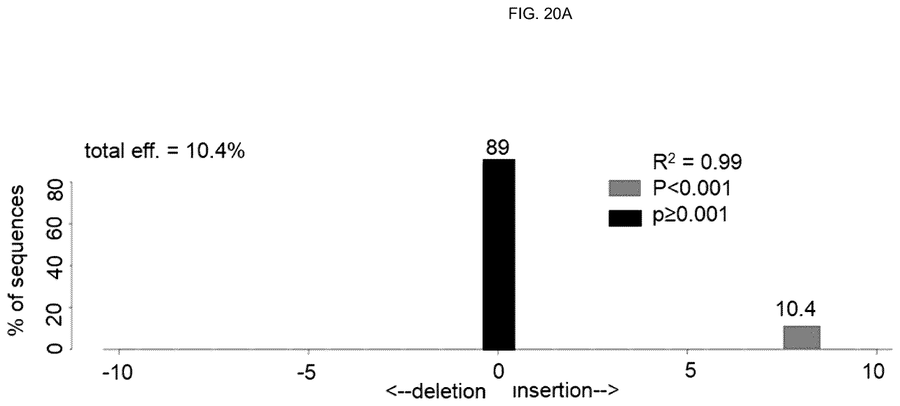

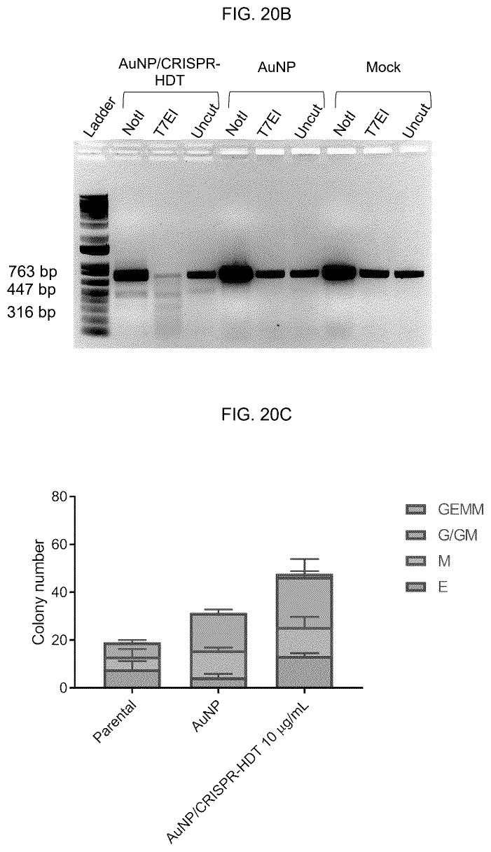

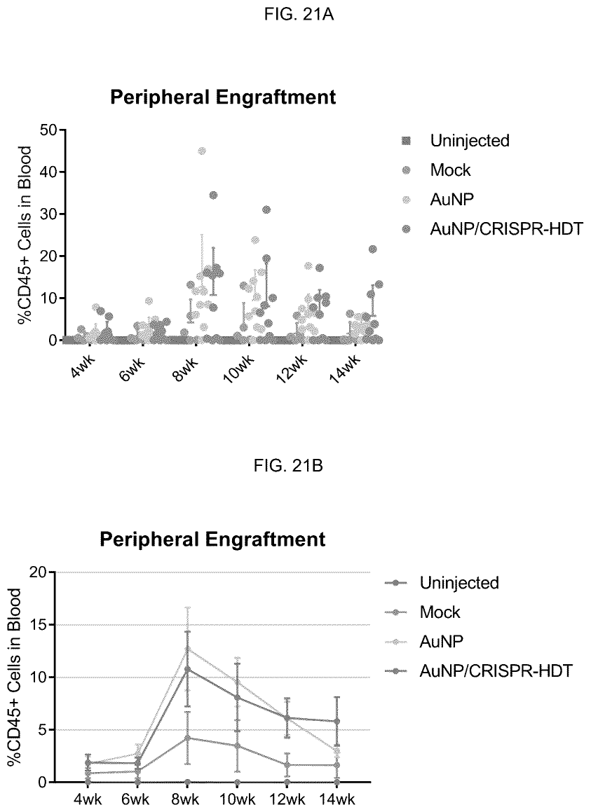

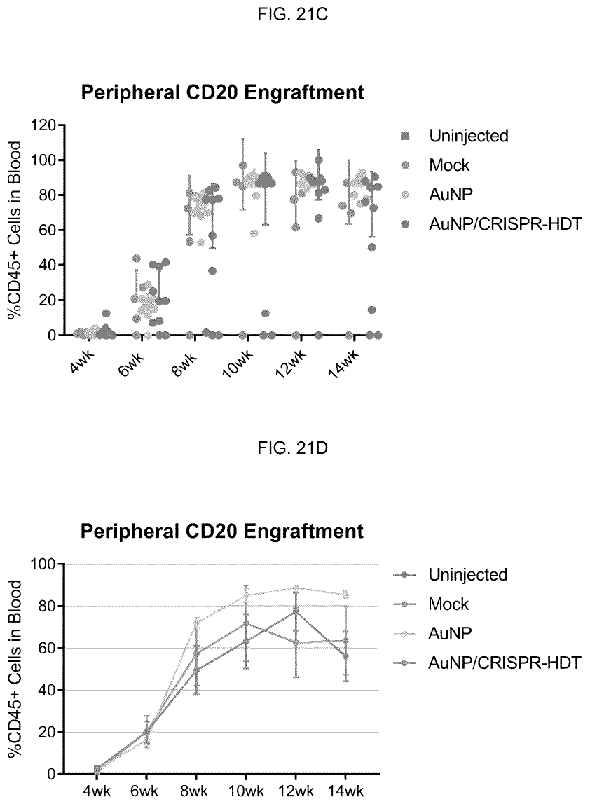

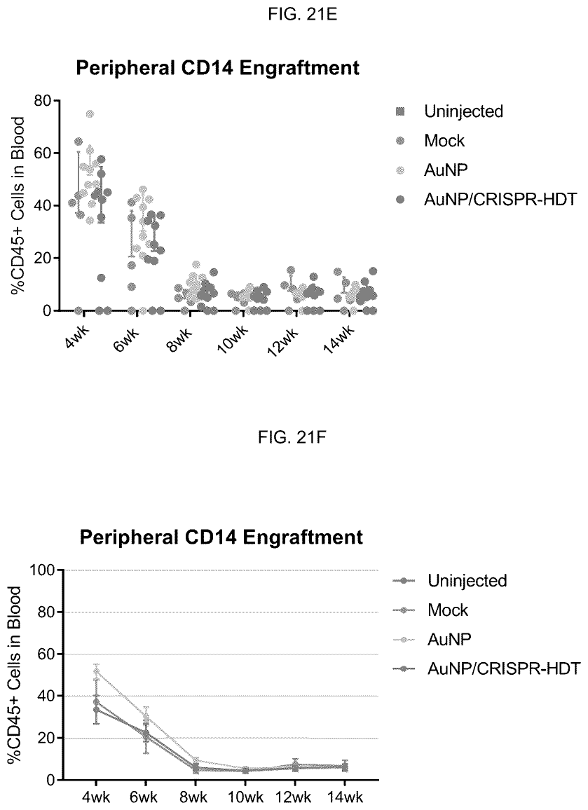

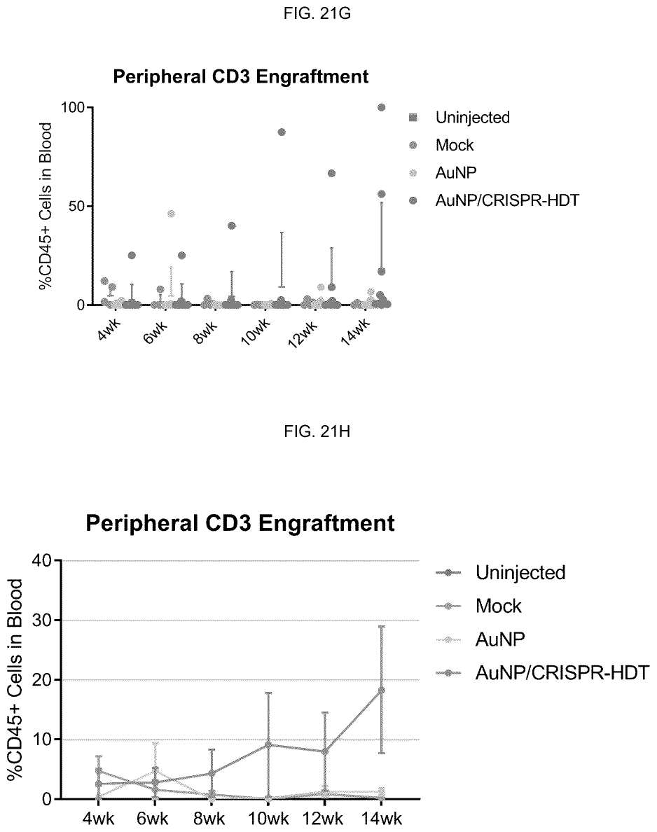

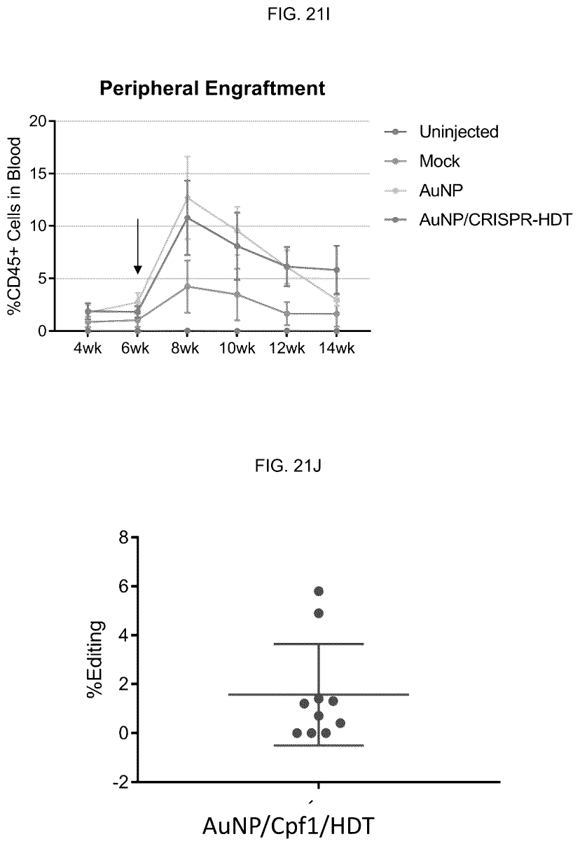

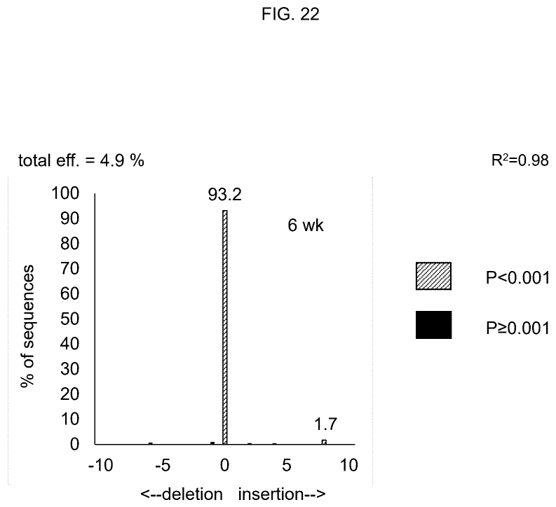

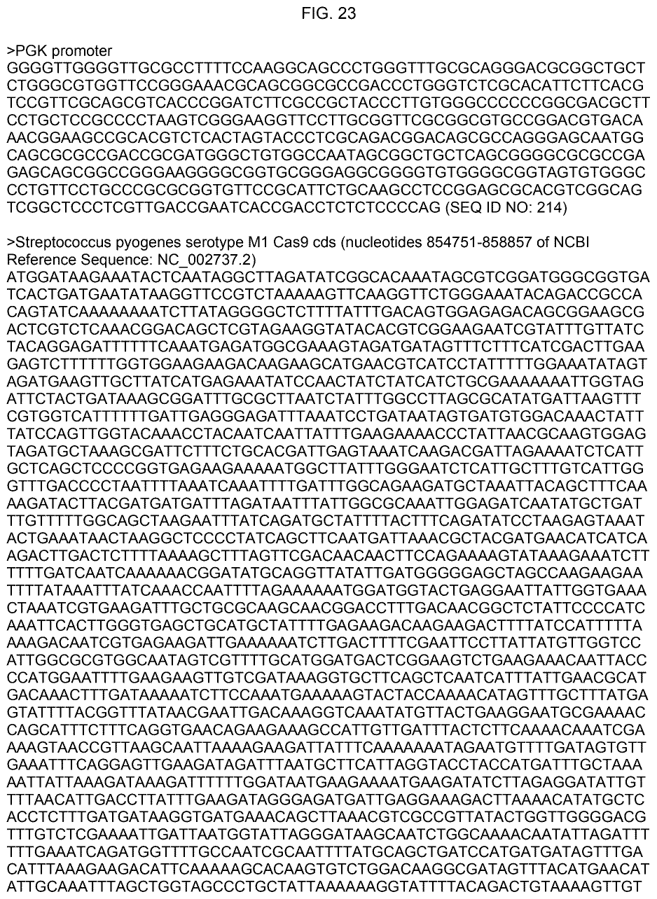

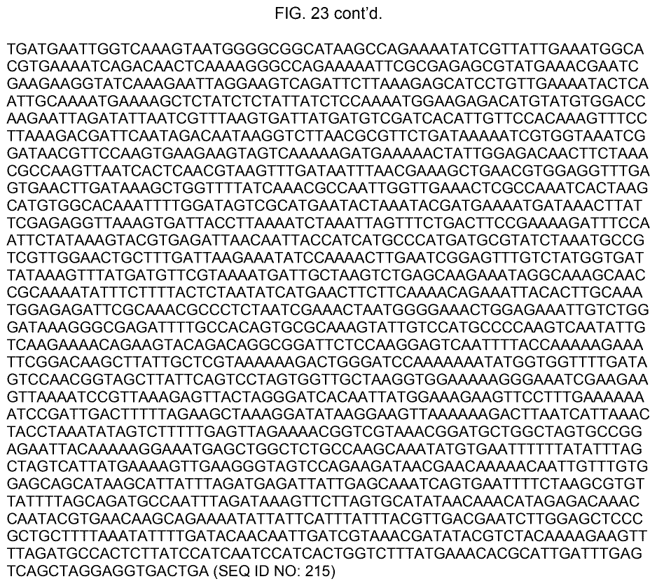

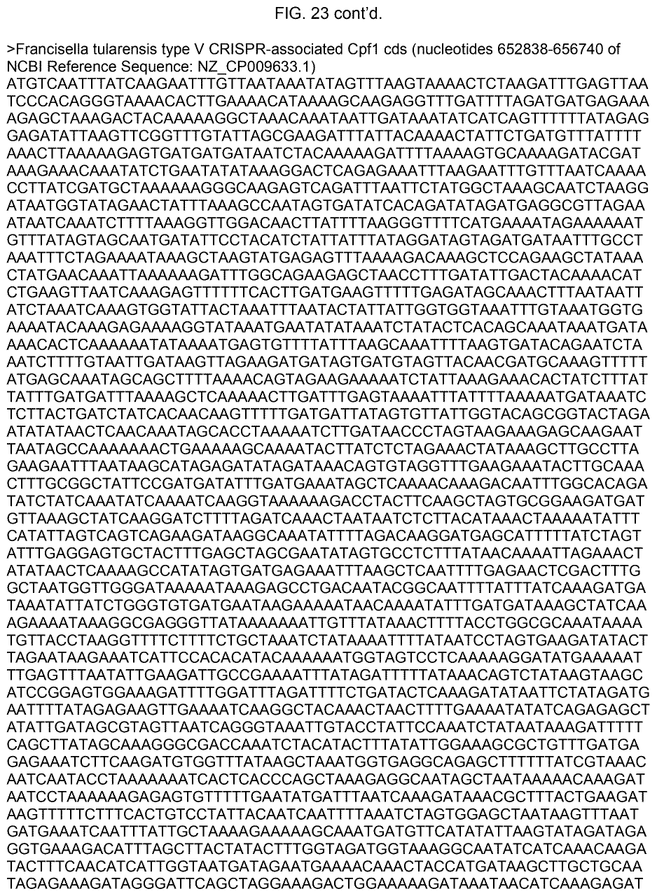

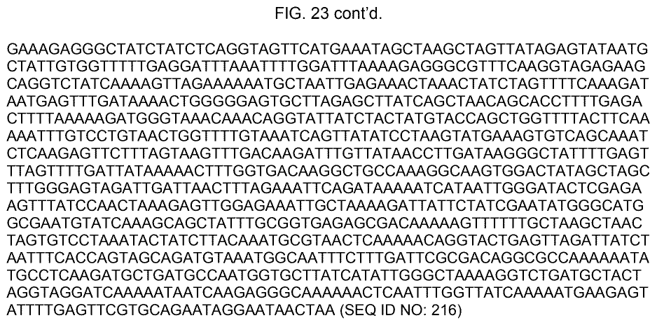

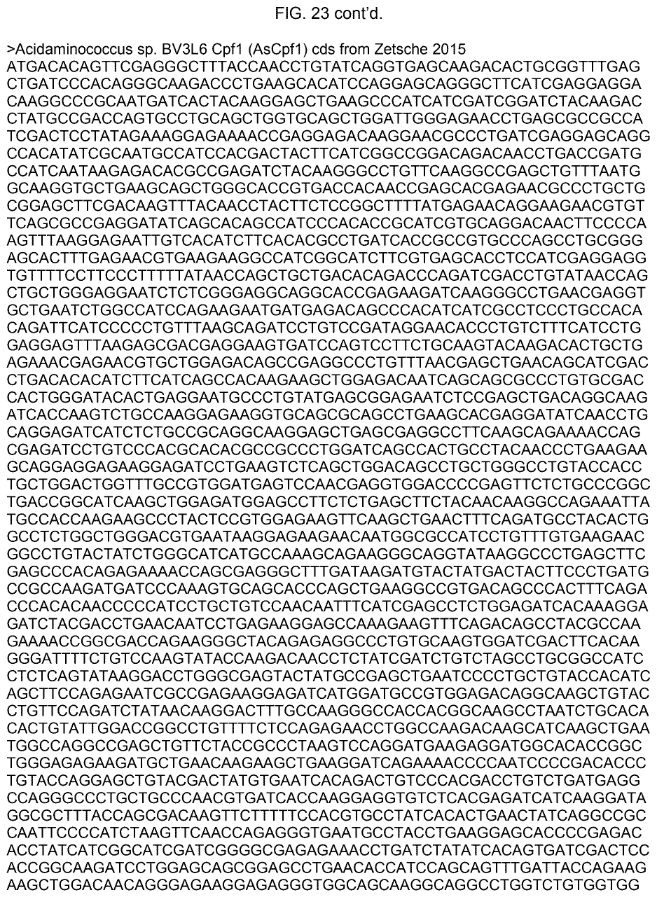

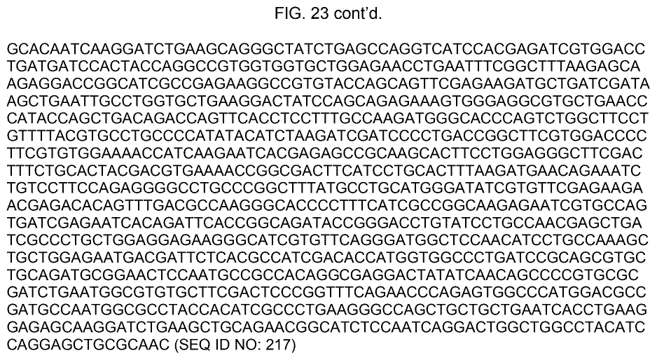

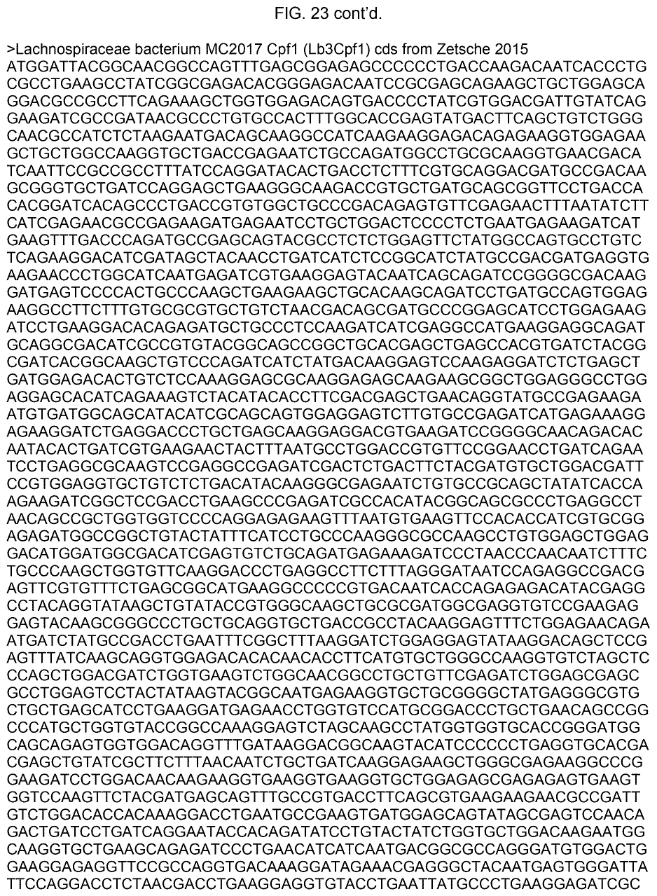

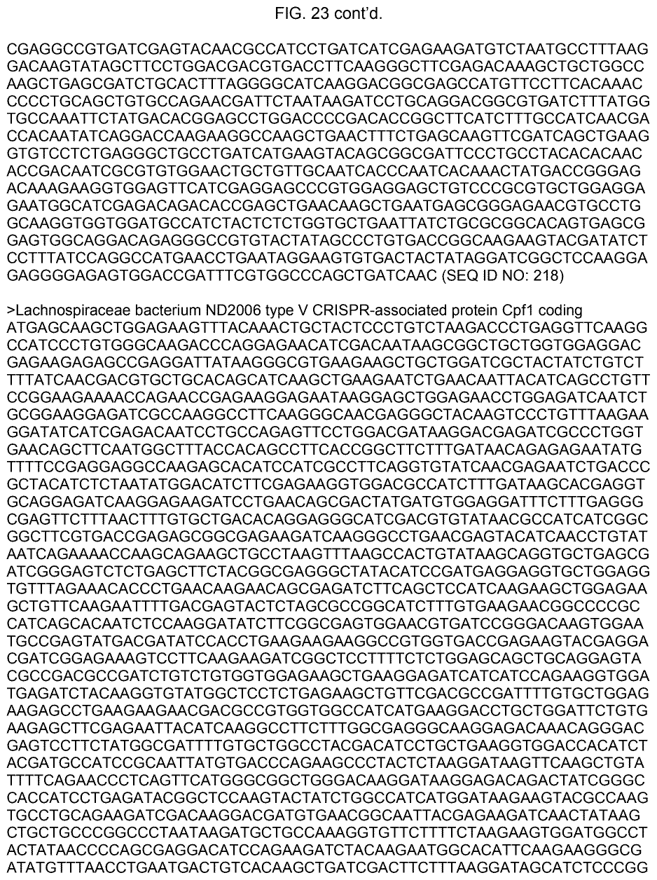

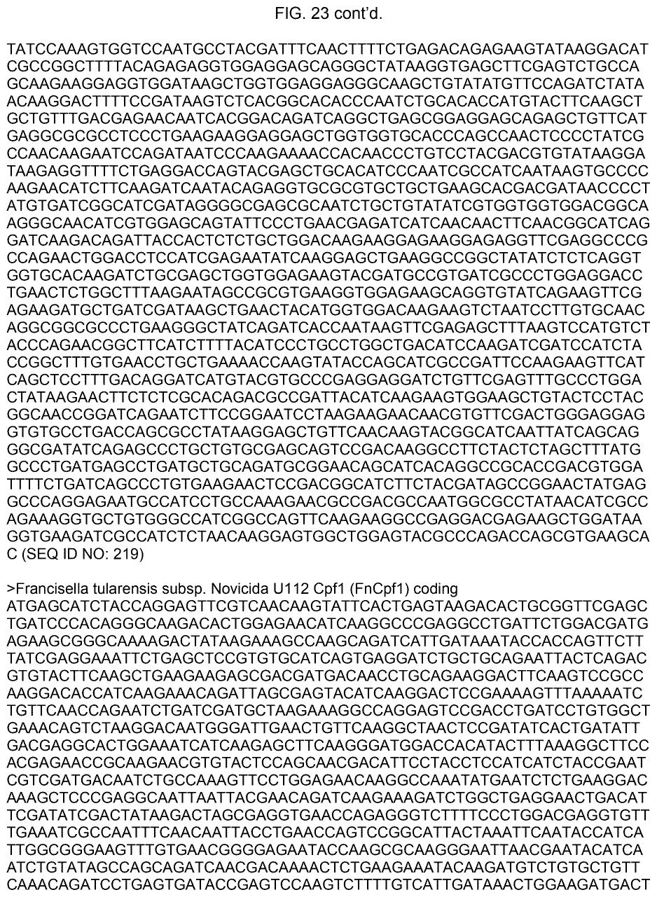

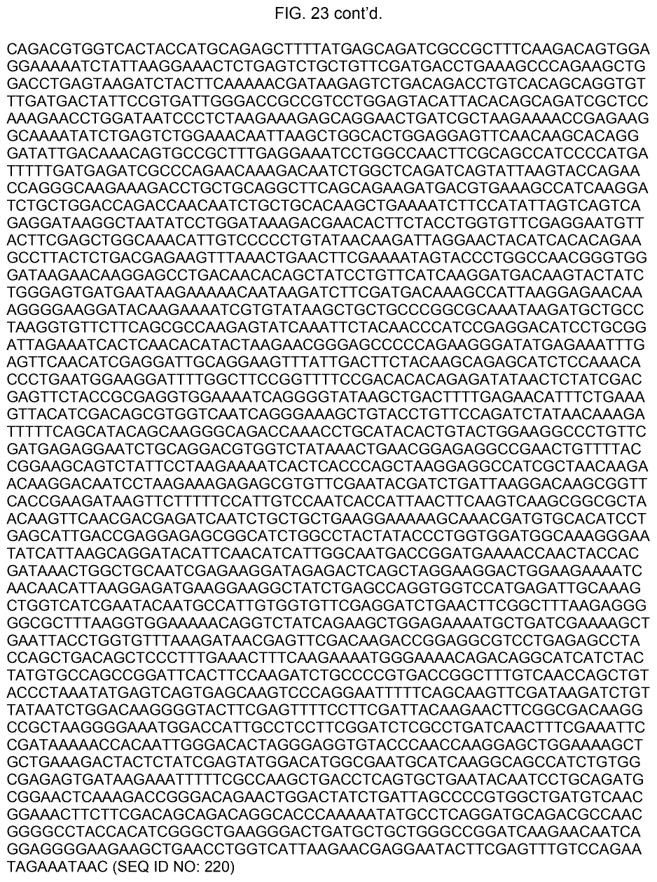

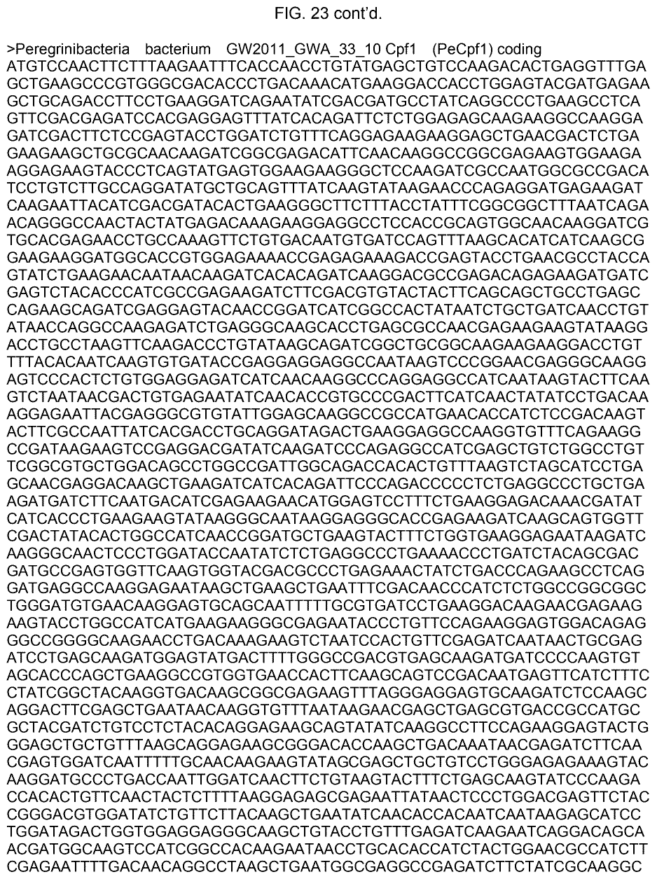

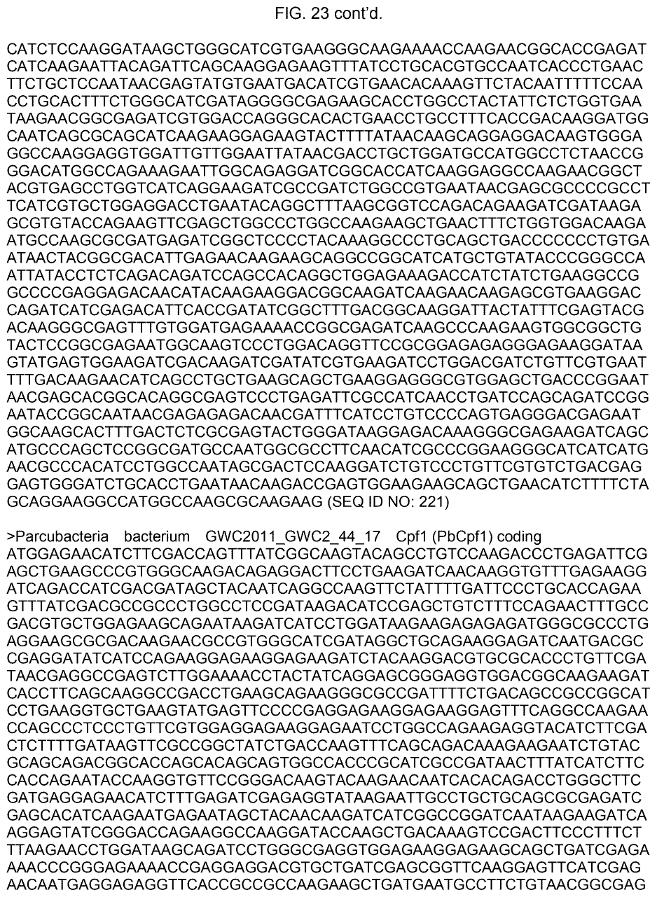

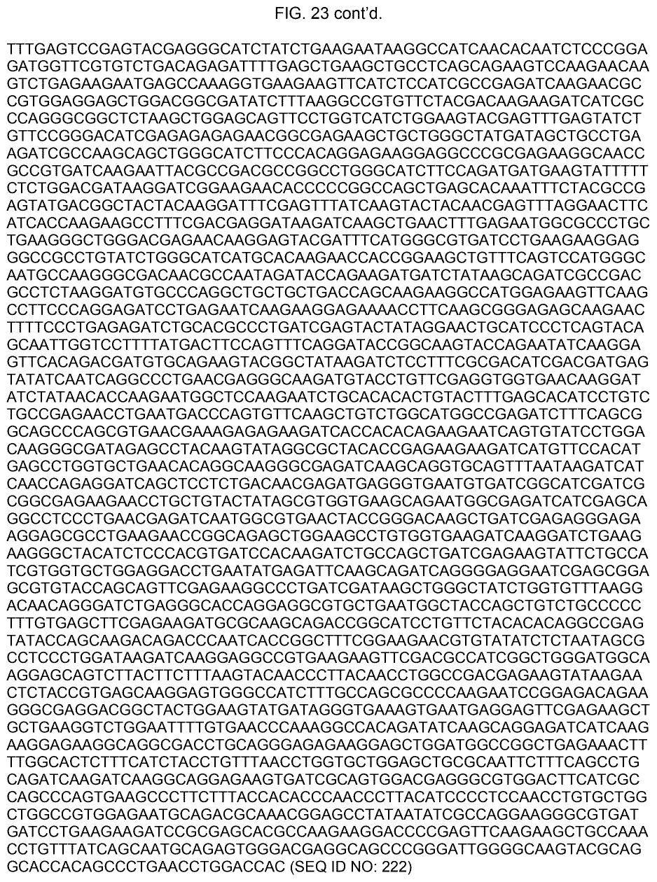

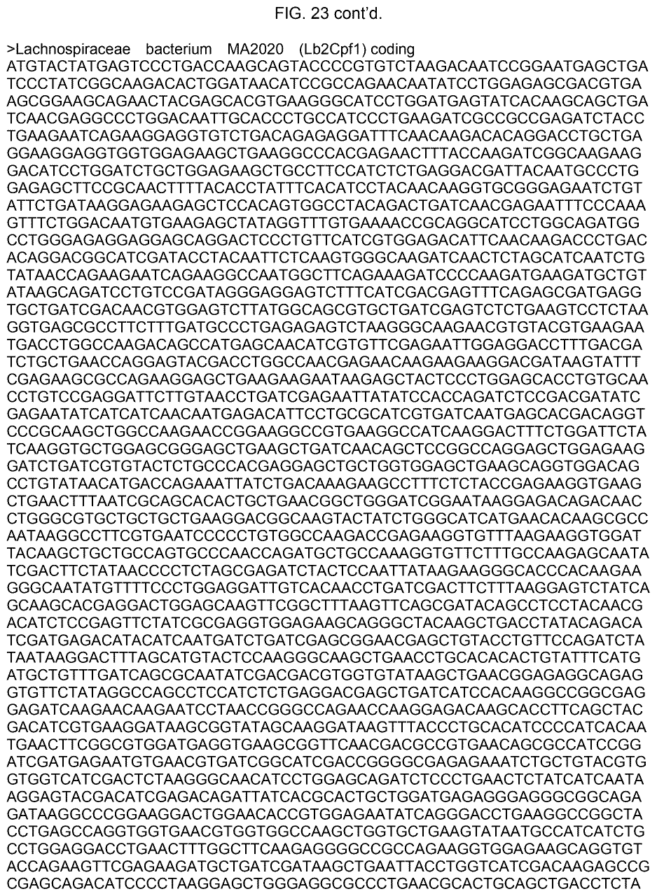

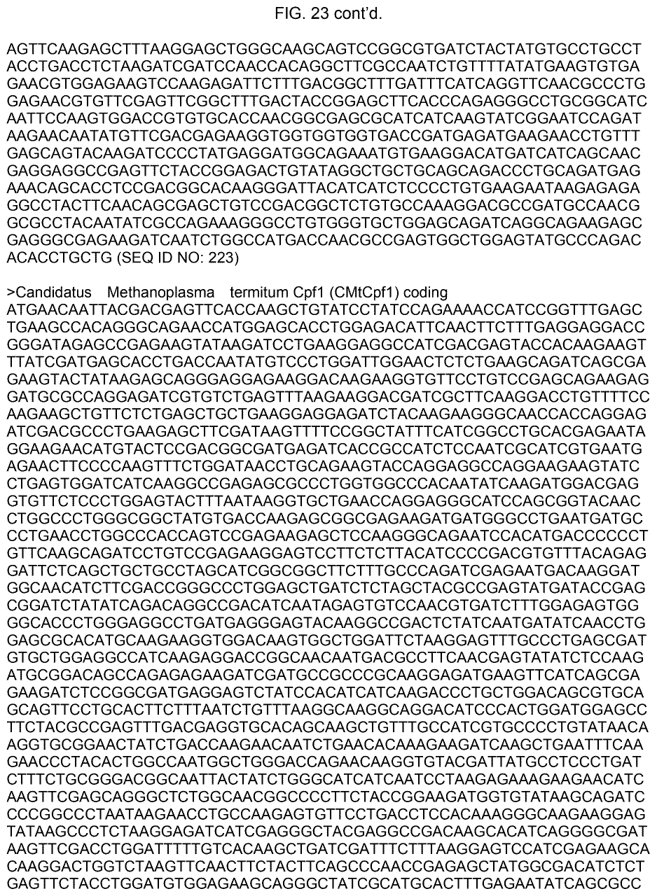

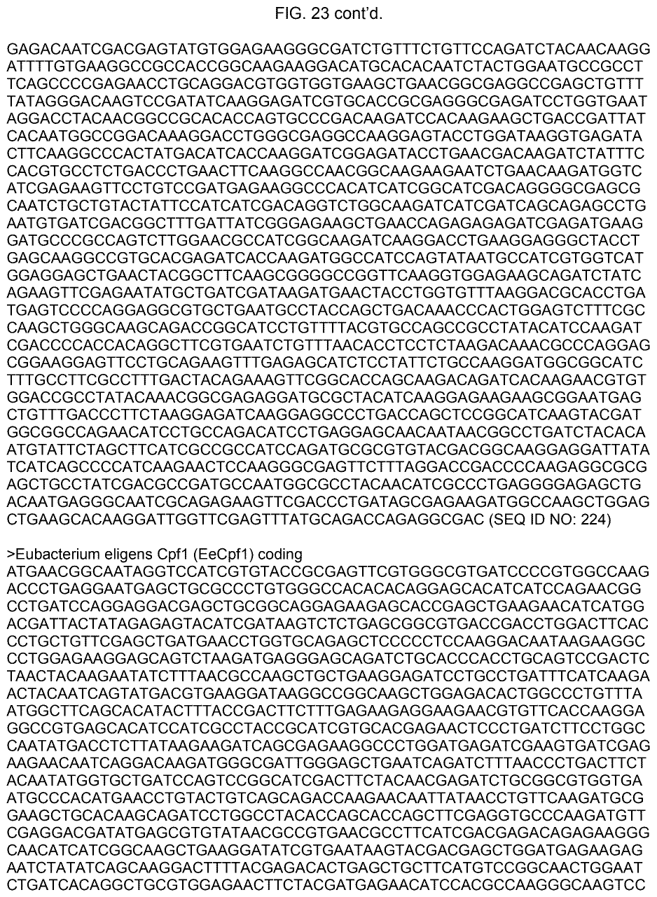

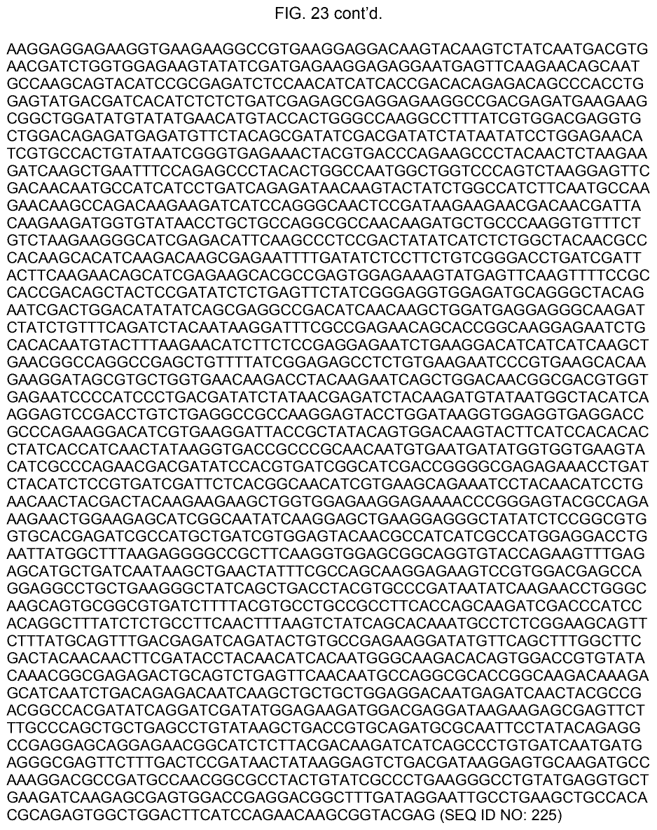

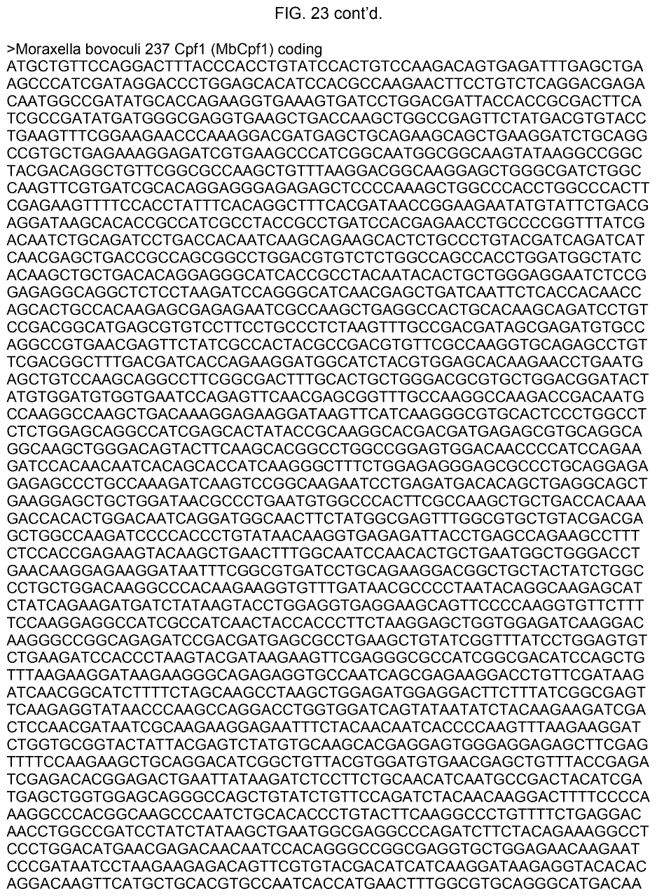

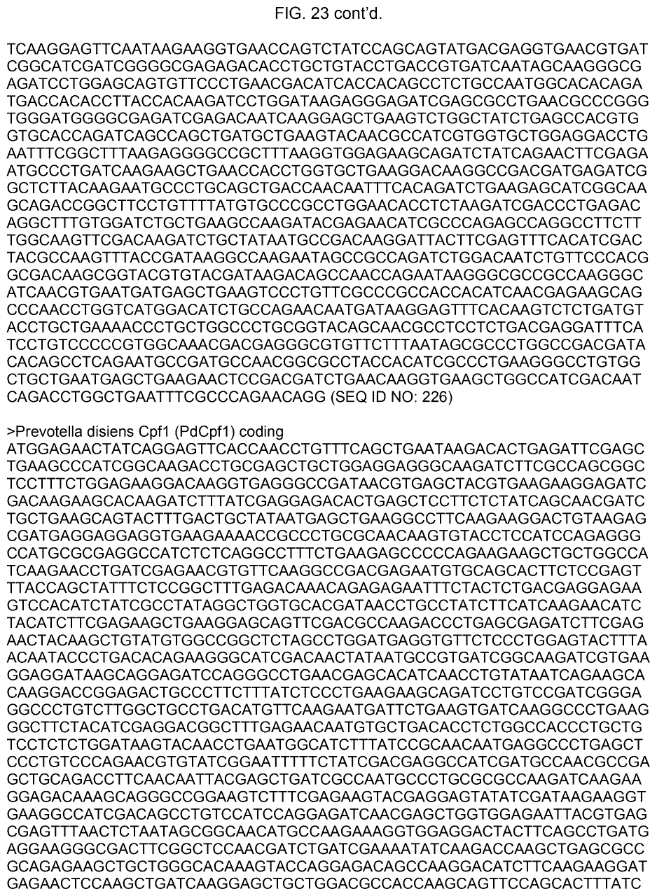

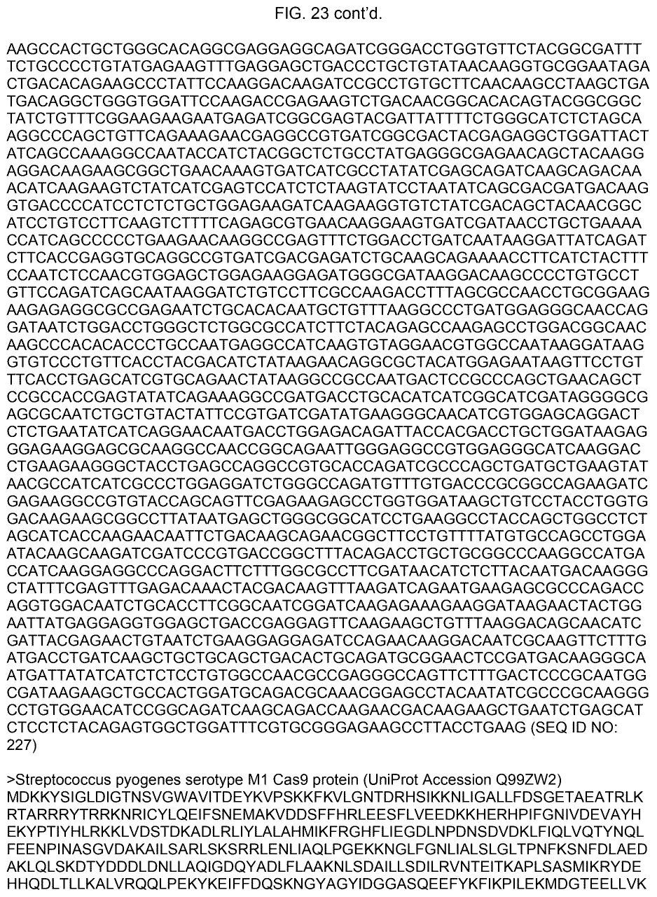

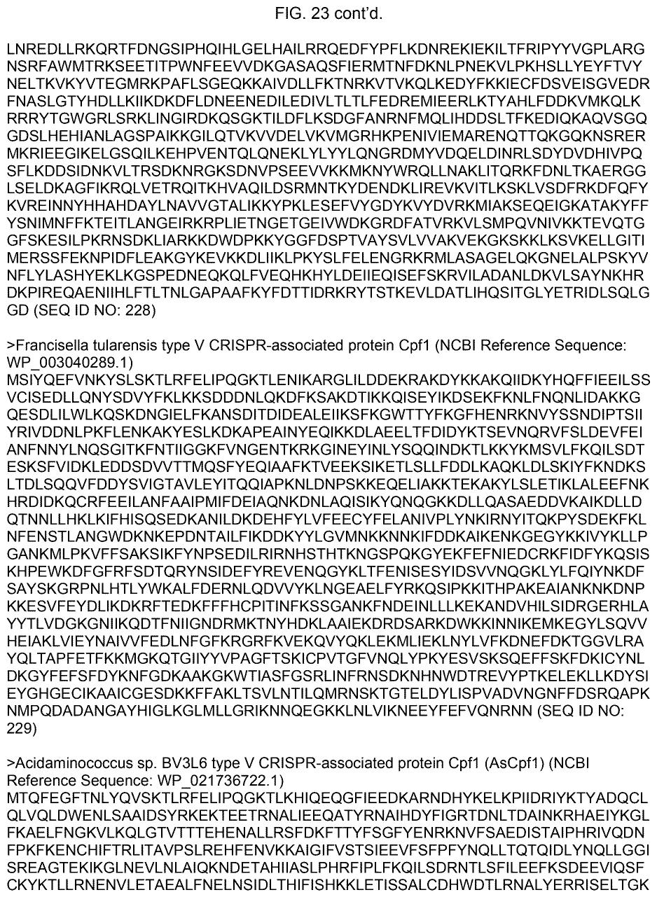

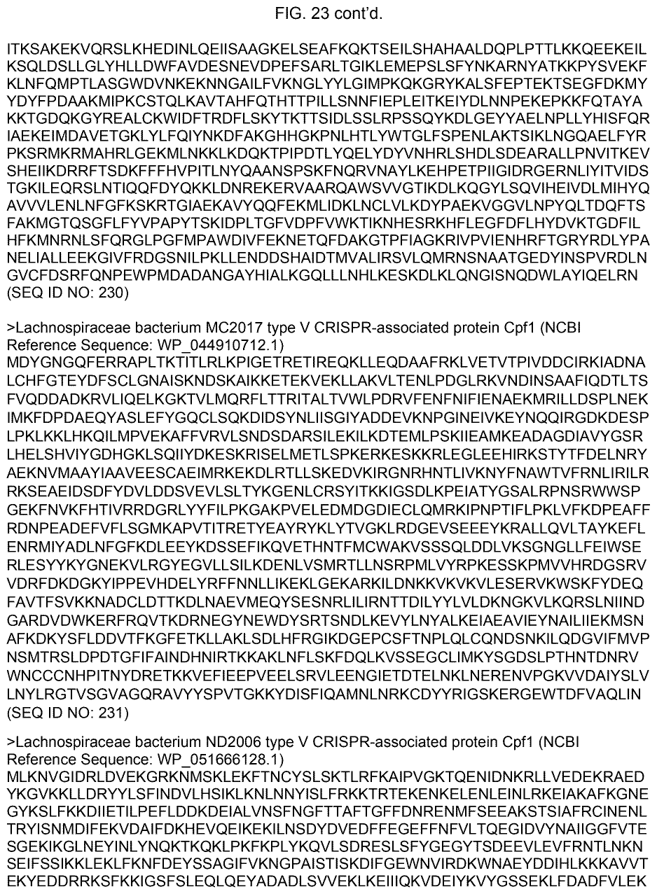

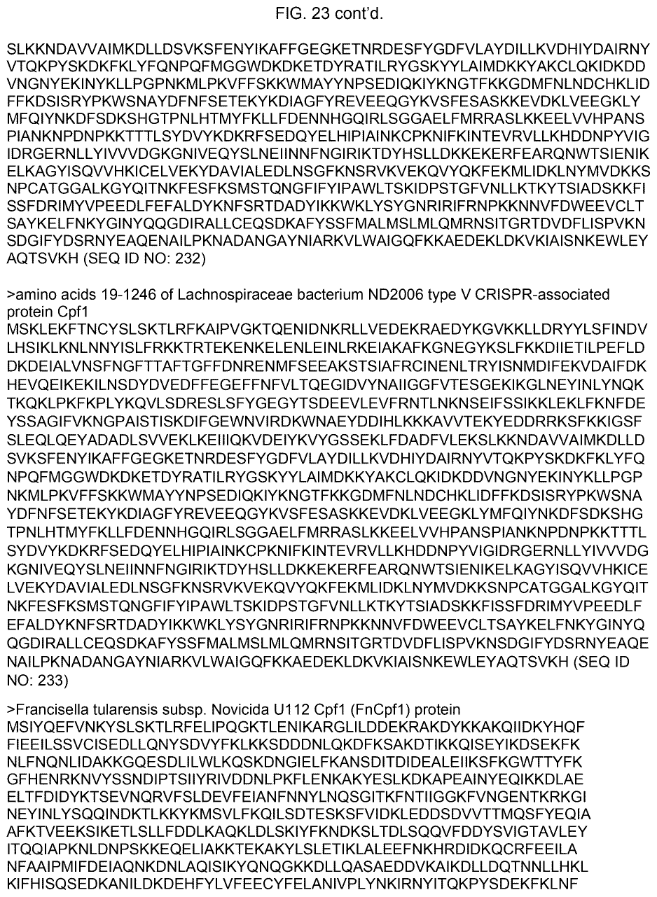

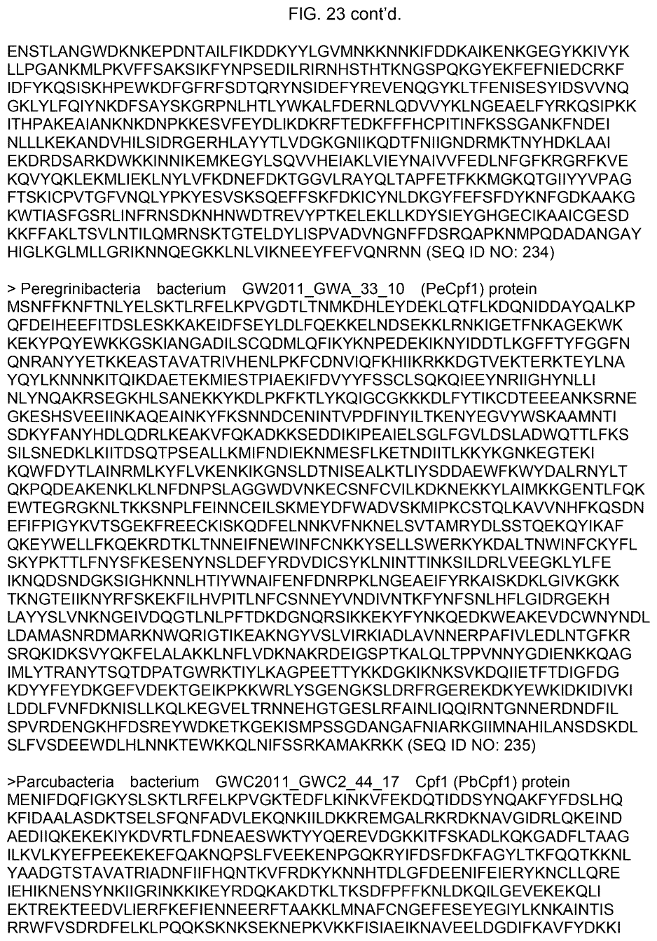

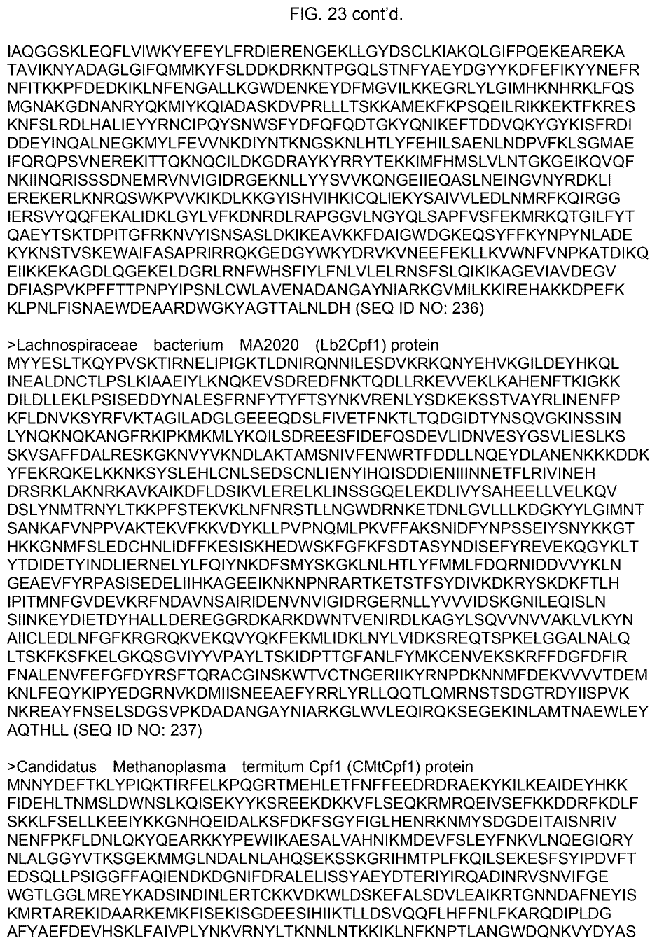

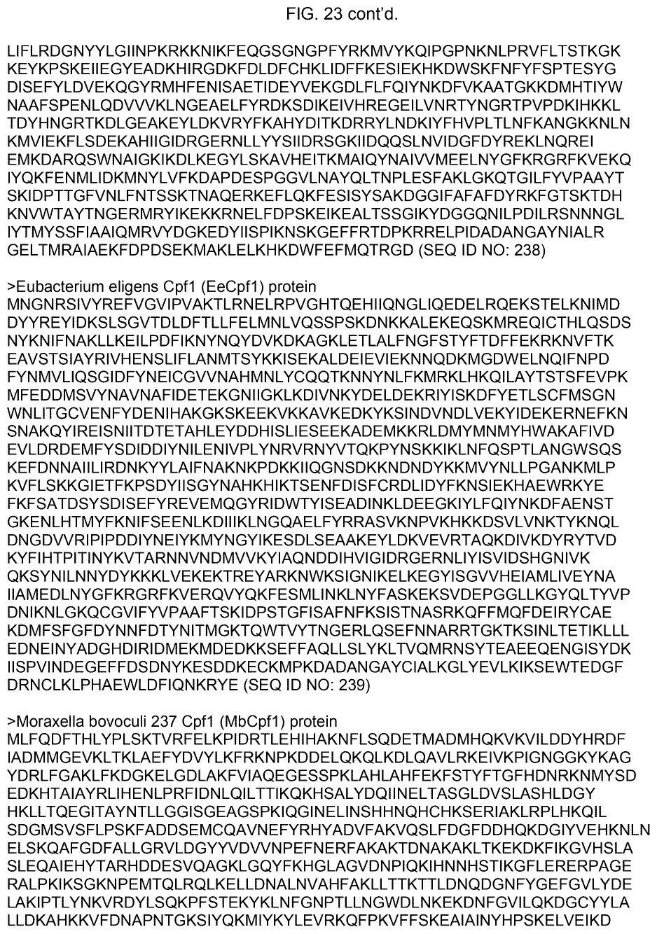

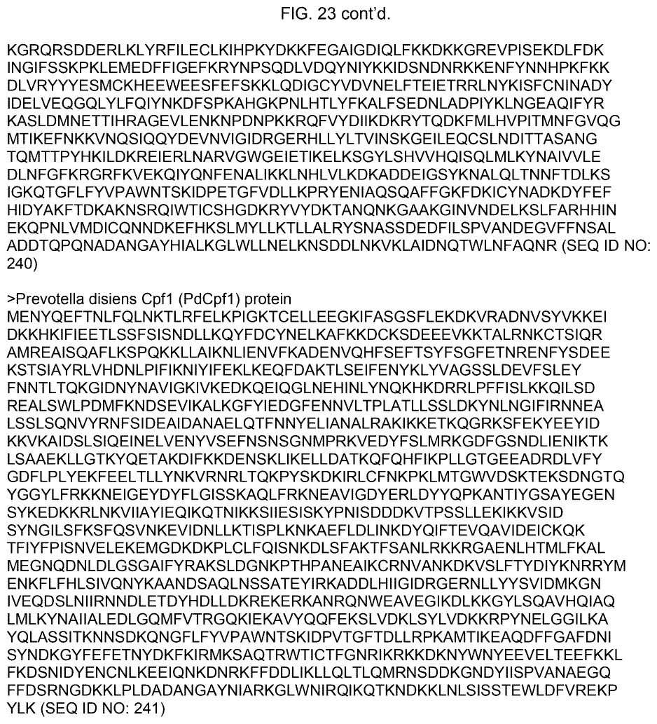

Confocal microscopy demonstrated that disclosed nanoformulations avoided lysosomal entrapment and successfully localized to the nucleus of CD34⁺ primary hematopoietic cells from healthy donors. Knock-in frequencies of up to 10% were demonstrated using a NotI restriction enzyme template with homology arm lengths of ±40 nucleotides to a CCR5 locus without cytotoxicity. Designing template to the non-target DNA strand yielded a higher homology directed repair (HDR) efficiency (FIG. 13), with clear 447 bp and 316 bp cut bands following digestion with NotI and T7EI enzymes (FIG. 20B). Direct comparison of Cpf1 and Cas9 nuclease activity at the same CCR5 target site demonstrated a Cpf1 bias for HDR and template knock-in over Cas9, which preferentially generated indels. Xenotransplantation of CRISPR Cpf1 nanoformulation-treated human CD34⁺ cells into immune deficient mice demonstrated an early increased trend in engraftment compared to non-treated cells, suggesting an unknown benefit of nanoformulation-treated HSPCs. The frequency of CCR5 genetically modified cell engraftment was the same as observed in culture, with 10% of human cells displaying NotI template addition in vivo.

(VI) Nanoparticle Compositions and Cell Formulations. Nanoparticles disclosed herein can be formulated into compositions for administration to subjects.

In particular embodiments, blood cells can be obtained from a subject or donor and genetically modified (e.g., within a GSH) before administration to the subject to treat a condition. Common sources of appropriate blood cells include mobilized peripheral blood samples, bone marrow samples, and/or umbilical cord blood.

Exemplary carriers for nanoparticle compositions and/or cell formulations include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof. In particular embodiments, nanoparticle compositions and/or cell formulations are administered to subjects as soon as reasonably possible following their initial formulation.

In particular embodiments, carriers can be supplemented with human serum albumin (HSA) or other human serum components or fetal bovine serum or other species serum components. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent component adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, nanoparticle compositions and/or cell formulations can include a local anesthetic such as lidocaine to ease pain at a site of injection.

Therapeutically effective amounts of nanoparticles within a composition can include at least 0.1% w/v or w/w particles; at least 1% w/v or w/w particles; at least 10% w/v or w/w particles; at least 20% w/v or w/w particles; at least 30% w/v or w/w particles; at least 40% w/v or w/w particles; at least 50% w/v or w/w particles; at least 60% w/v or w/w particles; at least 70% w/v or w/w particles; at least 80% w/v or w/w particles; at least 90% w/v or w/w particles; at least 95% w/v or w/w particles; or at least 99% w/v or w/w particles.

Therapeutically effective amounts of cells within cell-based formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

The nanoparticle compositions and/or cell formulations disclosed herein can be prepared for administration by, for example, injection, infusion, perfusion, or lavage.

In particular embodiments, it can be necessary or beneficial to freeze dry a nanoparticle composition and/or to cryopreserve a cell-based formulation. Such techniques are well known to those of ordinary skill in the art.

(VII) Exemplary Methods of Use. Hematopoietic stem cells (HSC) are stem cells that can give rise to all blood cell types such as the white blood cells of the immune system (e.g., virus-fighting T cells and antibody-producing B cells) and red blood cells. The therapeutic administration of HSC can be used to treat a variety of adverse conditions including immune deficiency diseases, blood disorders, malignant cancers, infections, and radiation exposure (e.g., cancer treatment, accidental, or attack-based). As examples, more than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Examples of diseases that can be treated using the nanoparticle compositions or cell formulations of the disclosure include a monogenetic blood disorder, hemophilia, Grave's Disease, rheumatoid arthritis, pernicious anemia, Multiple Sclerosis (MS), inflammatory bowel disease, systemic lupus erythematosus (SLE), Wiskott-Aldrich syndrome (WAS), chronic granulomatous disease (CGD), Battens disease, adrenoleukodystrophy (ALD) or metachromatic leukodystrophy (MLD), muscular dystrophy, pulmonary aveolar proteinosis (PAP), pyruvate kinase deficiency, Shwachmann-Diamond-Blackfan anemia, dyskeratosis congenita, cystic fibrosis, Parkinson's disease, Alzheimer's disease, amyotrophic lateral sclerosis (Lou Gehrig's disease), acute lymphoblastic leukemia (ALL), acute myelogenous leukemia (AML), agnogenic myeloid metaplasia, amegakaryocytosis/congenital thrombocytopenia, ataxia telangiectasia, β-thalassemia major, CLL, chronic myelogenous leukemia (CML), chronic myelomonocytic leukemia, common variable immune deficiency (CVID), complement disorders, congenital (X-linked) agammaglobulinemia, familial erythrophagocytic lymphohistiocytosis, Hodgkin's lymphoma, Hurler's syndrome, hyper IgM, IgG subclass deficiency, juvenile myelomonocytic leukemia, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, selective IgA deficiency, severe aplastic anemia, SCD, and/or specific antibody deficiency.

Particular embodiments include treatment of bacterial and/or parasitic infections. One exemplary parasite includes malaria-causing Plasmodium.

The compositions and formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). In particular embodiments, subjects are human patients. Nanoparticles described herein can be customized for any target while GSH sites are specific to humans and non-human primates.

Treating subjects includes delivering therapeutically effective amounts. Therapeutically effective amounts include those that provide effective amounts, prophylactic treatments, and/or therapeutic treatments.

An “effective amount” is the amount of a formulation necessary to result in a desired physiological change in a subject. Effective amounts are often administered for research purposes.

Assay for HSPC functionality. The most robust assay for HSPC functionality is the ability to reconstitute hematopoiesis in a conditioned recipient. In particular embodiments, human HSPC are xenotransplanted following electroporation of an optimal CRISPR/Cpf1 pair and ssODN combination into sub-lethally irradiated neonatal NOD/SCIDgamma^{null} (NSG) mice. The hematology, engraftment and persistence of GFP⁺ cells is then monitored by flow cytometry across blood cell lineages and time for 20 weeks after transplant. Genomic DNA (gDNA) is also isolated from blood, BM and spleen of transplanted animals at the time of necropsy for DNA barcode sequencing to determine the number of clones contributing to GFP⁺ cell hematopoiesis observed in vivo. Animals receiving HSPC modified with CRISPR/Cpf1 and ssODN are monitored for GFP⁺ cells by flow cytometry across blood cell lineages and time. Genomic DNA (gDNA) is isolated from blood, BM and spleen of transplanted animals at the time of necropsy for DNA barcode sequencing to determine the number of clones contributing to GFP⁺ cell hematopoiesis observed in vivo, in addition to more thorough evaluation of indel formation and persistence.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition.

A “therapeutic treatment” includes a treatment administered to a subject who displays symptoms or signs of a condition and is administered to the subject for the purpose of reducing the severity or progression of the condition.

The actual dose and amount of a therapeutic composition or formulation administered to a particular subject can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts can be administered through any appropriate administration route such as by, injection, infusion, perfusion, or lavage.

In particular embodiments, methods of the present disclosure can restore BM function in a subject in need thereof. In particular embodiments, restoring BM function can include improving BM repopulation with gene corrected cells as compared to a subject in need thereof not administered a therapy described herein. Improving BM repopulation with gene corrected cells can include increasing the percentage of cells that are gene corrected. In particular embodiments, the cells are selected from white blood cells and BM derived cells. In particular embodiments, the percentage of cells that are gene corrected can be measured using an assay selected from quantitative real time PCR and flow cytometry.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage φX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range of a reference level can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein.

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, restoring thymic output can include restoring the frequency of CD3+ T cells expressing CD45RA in PB to a level comparable to that of a reference level derived from a control population. In particular embodiments, restoring thymic output can include restoring the number of T cell receptor excision circles (TRECs) per 10⁶ maturing T cells to a level comparable to that of a reference level derived from a control population. The number of TRECs per 10⁶ maturing T cells can be determined as described in Kennedy D R et al. (2011) Vet Immunol Immunopathol 142: 36-48.

In particular embodiments, restoring normal T lymphocyte development includes restoring the ratio of CD4+ cells: CD8+ cells to 2. In particular embodiments, restoring normal T lymphocyte development includes detecting the presence of as TCR in circulating T-lymphocytes. The presence of as TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCRβ gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, a therapeutically effective treatment induces or increases production of hemoglobin; induces or increases production of beta-globin, or alpha-globin; and/or increases the availability of oxygen to cells in the body.

In particular embodiments, a therapeutically effective treatment increases blood cell counts, improves blood cell function, and/or increases oxygenation of cells.

In particular embodiments, a therapeutically effective treatment increases the production of coagulation/clotting factor VIII or coagulation/clotting factor IX, causes the production of normal versions of coagulation factor VIII or coagulation factor IX, reduces the production of antibodies to coagulation/clotting factor VIII or coagulation/clotting factor IX, and/or causes the proper formation of blood clots.

In particular embodiments, a therapeutically effective treatment causes the degradation of mucopolysaccharides in lysosomes, reduces, eliminates, prevents, or delays the swelling in various organs, including the head (exp. Macrosephaly), the liver, spleen, tongue, or vocal cords; reduces fluid in the brain; reduces heart valve abnormalities; prevents or dilates narrowing airways, reduces or prevent upper respiratory conditions like infections and sleep apnea; and/or reduces, eliminates, prevents, or delays the destruction of neurons and/or the symptoms associated with the destruction of neurons.

In particular embodiments, therapeutically effective amounts may provide function to immune and other blood cells, reduce or eliminate an immune-mediated condition; and/or reduce or eliminate a symptom of the immune-mediated condition.

In particular embodiments, particular methods of use include in the treatment of conditions where corrected cells have a selective advantage over non-corrected cells. For example, in FA and SCID, corrected cells have an advantage and only transducing the therapeutic gene into a “few” HSPCs is sufficient for therapeutic efficacy.

Additional methods of treatment can be found in International Patent Application PCT/US2016/014378, filed Jan. 21, 2016 and US Provisional Application Nos. 62/351,761, filed Jun. 17, 2016 and 62/428,994, filed Dec. 1, 2016, each of which is specifically incorporated herein in their entirety.

(VIII) Reference Levels Derived from Control Populations. Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 0-2 years) and non-immunocompromised status. In particular embodiments, a normal control population includes individuals that are age-matched to a test subject and non-immune compromised. In particular embodiments, age-matched includes, e.g., 0-6 months old; 0-1 year old; 0-2 years old; 0-3 years old; 10-15 years old, as is clinically relevant under the circumstances. In particular embodiments, a control population can include those that have an immune deficiency and have not been administered a therapeutically effective amount

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

(IX) Kits. The disclosure also provides kits containing any one or more of the elements disclosed in the methods and compositions herein. In particular embodiments, a kit can include guide RNA and a nuclease capable of cutting a target sequence in a blood cell GSH and an HDR template. Elements may be provided individually or in combinations, and may be provided in any suitable container, such as a vial, a bottle, a bag or a tube. In some embodiments, the kit includes instructions in one or more languages, for example in more than one language.

In particular embodiments, a kit includes one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from 7 to 10. In some embodiments, the kit includes a homologous recombination template polynucleotide.

Exemplary Embodiments: a gene editing sequence to target a genomic safe harbor disclosed herein (see, e.g., FIG. 1, 3, 4); a nanoparticle conjugated to gene editing sequences disclosed herein (see, e.g., FIG. 1, 3, 4); a CRISPR gene editing sequence modified at the 3′ end.
1. A method of genetically modifying a cell at a target sequence within chromosome 11 including SEQ ID NOs. 1-194, 197-208, 210, 213, 242, 245, 251, or 254 including: contacting the cell with a targeting element, a cutting element, and a homology-directed repair template wherein the contacting results in (i) cutting within the target sequence; and (ii) homology-directed repair (HDR).
2. The method of embodiment 1, wherein the targeting element, the cutting element and the homology-directed repair template are part of a CRISPR gene editing system, a meganuclease gene editing system, a zinc finger nuclease (ZFN) gene editing system, or a transcription activator-like effector-based nuclease (TALEN) gene editing system.
3. The method of embodiment 1 or 2, wherein the targeting element is crRNA that hybridizes to one of SEQ ID NOs. 1-194, 197-208, 210, 213, 242, 245, 251, or 254.
4. The method of any of embodiments 1-3, wherein the cutting element is Cpf1 or Cas 9.
5. The method of embodiment 4, wherein the Cpf1 or Cas9 includes a sequence selected from SEQ ID NOs: 215-241.
6. The method of embodiment 4, wherein the Cpf1 is a variant of a Cpf1 selected from SEQ ID NOs: 216-227, or 229-241.
7. The method of any of embodiments 1-6, wherein the homology-directed repair template includes homology arms to the target sequence, and wherein the homology-directed repair template is part of a donor template further including a therapeutic gene that results in expression of a therapeutic gene product.
8. The method of embodiment 7, wherein the therapeutic gene or therapeutic gene product is selected from skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C90RF72, α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, Fancl, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TAL1, TCL3, TFPI, thrombospondin, thymidine kinase, TNF, TP53, trk, T-VEC, VEGF, VHL, WT1, WT-1, YES, zac1, iduronidase, IDS, GNS, HGSNAT, SGSH, NAGLU, GUSB, GALNS, GLB1, ARSB, HYAL1, F8, F9, HBB, CYB5R3, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.
9. The method of embodiment 7 or 8, wherein the homology arms are 40 nucleotides (nt)-1000 nt.
10. The method of any of embodiments 1-9, wherein the targeting element and the cutting element are separate molecules or are part of a single dual-purpose molecule.
11. The method of any of embodiments 1-10, wherein the targeting element and the cutting element are coupled to a nanoparticle.
12. The method of embodiment 11, wherein the nanoparticle includes a gold nanoparticle.
13. The method of any of embodiments 1-12 wherein the targeting element and/or the cutting element are conjugated to a spacer.
14. The method of embodiment 13 wherein the spacer includes a thiol modification.
15. The method of embodiment 14 wherein the thiol modification is covalently linked to the surface of the nanoparticle.
16. The method of any of embodiments 13-15 wherein the targeting element includes a 3′ end and a 5′ end, and wherein the 3′ end is conjugated to the spacer.
17. The method of any of embodiments 11-16 wherein the nanoparticle is associated with at least two layers wherein the first layer includes the targeting element and the second layer includes a donor template including a therapeutic gene and homology-directed repair templates; and wherein at least a portion of the second layer is farther from the surface of the nanoparticle than the first layer.
18. The method of any of embodiments 11-17, wherein the nanoparticle is coupled to a targeting molecule.
19. The method of embodiment 18, wherein the targeting molecule includes a CD34 binding domain or a CD90 binding domain.
20. The method of any of embodiments 1-19, wherein the cell includes a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), a endothelial cell (EC), a stromal cell, and/or a bone marrow fibroblast.
21. The method of embodiment 20, wherein the blood cell includes a CD34⁺CD45RA⁻CD90⁺ HSC.
22. The method of embodiment 20 or 21, wherein the blood cell is a human blood cell.
23. The method of any of embodiments 1-22, wherein the cutting results in a staggered DNA double strand break with a 2-4-nt 5′ overhangs.
24. The method of any of embodiments 1-23 utilizing at least one of the following target sequence crRNA pairs: (i) target: SEQ ID NO: 132/crRNA: SEQ ID NO: 195; (ii) target: SEQ ID NO: 108/crRNA: SEQ ID NO: 196; (iii) target: SEQ ID NO: 203/crRNA: SEQ ID NO: 209; (iv) target: SEQ ID NO: 210/crRNA: SEQ ID NO: 211; (v) target: SEQ ID NO: 242/crRNA: SEQ ID NO: 244; and (vi) target: SEQ ID NO: 251/crRNA: SEQ ID NO: 253.
25. A nanoparticle associated with at least two active layers wherein the first layer includes a DNA targeting element and a cutting element and wherein the second layer includes a donor template including a therapeutic gene and homology-directed repair templates; and wherein at least portions of the second layer are farther from the surface of the nanoparticle than the first layer.
26. The nanoparticle of embodiment 24 wherein the targeting element hybridizes to one of SEQ ID NOs. 1-194, 197-208, 210, 212, 213, 242, 245, 251, 254, 258, or 263. 27. The nanoparticle of embodiment 25 or 26, wherein the DNA targeting element is crRNA with a 3′ end and a 5′ end, wherein the 3′ end is conjugated to a spacer.
28. The nanoparticle of any of embodiments 25-27 wherein the DNA targeting element is crRNA with a 3′ end and a 5′ end, wherein the 5′ end is conjugated to the cutting element.
29. The nanoparticle of embodiment 27 or 28 wherein the spacer includes a thiol modification that is covalently linked to the surface of the nanoparticle.
30. The nanoparticle of any of embodiments 25-29 wherein the cutting element is Cpf1 or Cas 9.
31. The nanoparticle of any of embodiments 27-30, wherein the crRNA includes SEQ ID NOs: 195, 196, 209, 211, 244, 253, 260, or 264.
32. The nanoparticle of embodiments 30 or 31, wherein the Cpf1 or Cas9 includes a sequence selected from SEQ ID NOs: 215-241.
33. The nanoparticle of any of embodiments 30-32, wherein the Cpf1 includes a variant of a Cpf1 selected from SEQ ID NOs: 216-227, or 229-241.
34. The nanoparticle of any of embodiments 25-33, wherein the nanoparticle is a gold nanoparticle.
35. The nanoparticle of any of embodiments 25-34, wherein the nanoparticle is coupled to a targeting molecule.
36. The nanoparticle of embodiment 35, wherein the targeting molecule includes a CD34 binding domain or a CD90 binding domain.
37. A therapeutic formulation including a nanoparticle of any of embodiments 24-35.
38. A cell genetically-modified by a method of any of embodiments 1-24 or a nanoparticle of any of embodiments 25-36.
39. The cell of embodiment 38, wherein the cell is a hematopoietic stem cell (HSC), a hematopoietic progenitor cell (HPC), a hematopoietic stem and progenitor cell (HSPC), a T cell, a natural killer (NK) cell, a B cell, a macrophage, a monocyte, a mesenchymal stem cell (MSC), a white blood cell (WBC), a mononuclear cell (MNC), a endothelial cell (EC), a stromal cell, and/or a bone marrow fibroblast.
40. The cell of embodiment 38 or 39, wherein the cell is a CD34⁺CD45RA⁻CD90⁺ HSC.
41. The cell of any of embodiments 38-40, wherein the cell is a human blood cell.
42. A therapeutic formulation including a cell of any of embodiments 38-41. 43. A method of providing a therapeutic nucleic acid sequence to a patient in need thereof including administering a therapeutic formulation of embodiment 37 and/or 42, to the patient thereby providing a therapeutic nucleic acid sequence to the patient.

Variants of protein and/or nucleic acid sequences disclosed herein can also be used. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

“% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

In particular embodiments, variant proteins include conservative amino acid substitutions. In particular embodiments, a conservative amino acid substitution may not substantially change the structural characteristics of the reference sequence (e.g., a replacement amino acid should not tend to break a helix that occurs in the reference sequence, or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (lie), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and lie. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, lie, Val, and Cys; and large aromatic residues: Phe, Tyr, and Trp. Additional information is found in Creighton (1984) Proteins, W. H. Freeman and Company.

As indicated previously, in particular embodiments, variants include engineered Cpf1s. For example, US 2018/0030425 describes engineered Cpf1 nucleases from Lachnospiraceae bacterium ND2006 and Acidaminococcus sp. BV3L6 with altered and improved target specificity. Particular variants include Lachnospiraceae bacterium ND2006 of SEQ ID NO: 232, e.g., at least including amino acids 19-1246 of SEQ ID NO: 232, with mutations (i.e., replacement of the native amino acid with a different amino acid, e.g., alanine, glycine, or serine), at one or more of the following positions: S203, N274, N278, K290, K367, K532, K609, K915, Q962, K963, K966, K1002, and/or S1003 of SEQ ID NO: 232 (or at positions analogous thereto, e.g., S185, N256, N260, K272, K349, K514, K591, K897, Q944, K945, K948, K984, and/or S985 of SEQ ID NO: 233). SEQ ID NO: 233 is identical to SEQ ID NO: 232 but lacks the first 18 amino acids. Particular Cpf1 variants can also include Acidaminococcus sp. BV3L6 Cpf1 (AsCpf1) of SEQ ID NO: 230, e.g., at least comprising amino acids 1-1307 of SEQ ID NO: 230, with mutations (i.e., replacement of the native amino acid with a different amino acid, e.g., alanine, glycine, or serine (except where the native amino acid is serine)), at one or more of the following positions: N178, S186, N278, N282, R301, T315, S376, N515, K523, K524, K603, K965, Q1013, Q1014, and/or K1054 of SEQ ID NO: 230. In particular embodiments, engineered Cpf1 variants include eCfp1.

Other Cpf1 variants include Cpf1 homologs and orthologs of the Cpf1 polypeptides disclosed in Zetsche et al. (2015) Cell 163: 759-771 as well as the Cpf1 polypeptides disclosed in U.S. 2016/0208243. Examples of Cpf1 sequences include SEQ ID NOs: 216-227 and 229-241 disclosed herein.

Other engineered Cpf1 variants are known to those of ordinary skill in the art and included within the scope of the current disclosure (see, e.g., WO/2017/184768).

### Exemplary Manufacturing Embodiment.

Exemplary Manufacturing Embodiment.

| Parameter | Disclosed Embodiment |
|---|---|
| Size of AuNP Core | 15 nm |
| AuNP Synthesis Method | Turkevich (1951) |
| Starting solution | 0.25 mM chloroauric acid (HAuCl₄) |
| 1st synthesis step | Bring above solution to boiling point and |
| | reduce by adding 3.33% sodium citrate |
| | (Na3C6H5O7) while stirring vigorously |
| | (700 rpm) under a reflux system |
| 2nd synthesis step | Reduce by adding 3.33% sodium citrate |
| | (Na₃C₆H₅O₇) while stirring vigorously |
| | (700 rpm) under a reflux system |
| Cleanup step | Wash AuNPs 3× |
| Initial Resuspension | Rnase free molecular grade water (H₂O) |
| First Loading Step | 10 micrograms/mL AuNP added to crRNA |
| | (Cpf1/Cas12a) or crRNA + tracrRNA |
| | (Cas9) solution at a weight/weight ratio |
| | of 0.5 |
| Second Loading Step | 10 mM Citrate buffer (pH 3.0) added and |
| | mixed for 5 min. Nanoconjugates are |
| | centrifuged at 20000 × g for 20 minutes |
| | at room temperature and re-dispersed in |
| | 0.9% sodium choloride. |
| Third Loading Step | Add nuclease protein (Cpf1/Cas12a or |
| | Cas9) to nanoconjugate solution at a |
| | weight/weight ratio of 0.6 |
| Fourth Loading Step | Add 0.005% branched polyethylenimine |
| | (2000 MW) and mix by pipetting. |
| Fifth Loading Step | Add single stranded DNA template |
| | (ssODN) to nanoconjugates in a weight |
| | to weight ratio of 1.0 |
| Final Resuspension | RNase free water |
| Guide RNA Loaded | Guide RNA (crRNA) with the following |
| | modifications: For Cpf1 (Cas12a): 1. 3′ |
| | 18-atom oligo ethylene glycol (OEG) |
| | spacer (iSp18) 2. 3′ terminal thiol For |
| | Cas9: (unmodified tracrRNA) 1. 5′ |
| | 18-atom oligo ethylene glycol (OEG) |
| | spacer (iSp18) 2. 5′ terminal thiol |
| Nuclease Loaded | Cpf1 (Cas12a), Cas9, or Mega-TAL |
| ssODN Loaded | Unmodified homology-directed template |
| | with symmetric or asymmetric homolgy |
| | arms of any length, up to a total of 3 |
| | kilobases in total |
| Final actual size of fully | 25-30 nm |
| loaded AuNP | |
| Final hydrodynamic size | 176 nm |
| of fully loaded AuNP | |

Example 1. Synthesizing Gold Nanoparticle Cores. Gold nanoparticles (AuNPs) of 15 nm size range were synthesized by Turkevich's method with slight modification. Turkevich, et al., (1951). Discussions of the Faraday Society 11(0): 55-75.). 0.25 mM Chloroauric acid solution was brought to the boiling point and reduced by adding 3.33% sodium citrate solution and stirred vigorously under reflux system for 10 min. Synthesized nanoparticles were washed three times and re-dispersed in highly pure water.

Cpf1 and Cas9 Guide RNA Structures. Single Cpf1 guide RNA was ordered from commercial source, Integrated DNA Technologies; IDT), with two custom modifications on the 3′ end. The first modification included an 18-atom oligo ethylene glycol (OEG) spacer (iSp18), and the second modification included a thiol modification. The OEG spacer (e.g. polyethylene glycol (PEG) or hexaethylene glycol (HEG), etc.), was at a ratio of 1 per oligonucleotide and served to prevent electrostatic repulsion between oligonucleotides. While an 18-atom spacer was used, other lengths are also appropriate. The thiol modification was also added at a ratio of 1 per oligonucleotide and served as the basis for covalent interactions to bind the oligonucleotide to the surface of the gold nanoparticle.

(SEQ ID NO: 260)
5′-/AltR1/rUrA rArUrU rUrCrU rArCrU rCrUrU
rGrUrA rGrArU rCrArC rCrCrG rArUrC rCrArC
rUrGrG rGrGrA rGrCrA /iSp18//3ThioMC3-D/-3′

For cas9, a two-part guide system including tracrRNA and crRNA was used. crRNA for Cas9 was ordered from IDT with the same 18 spacer-thiol modifications as above, but on the 5′ end.

(SEQ ID NO: 264)
5′-/5ThioMC6-D//iSp18/rCrA rCrCrC rGrArU
rCrCrA rCrUrG rGrGrG rArGrC rGrUrU rUrUrA
rGrArG rCrUrA rUrGrC rU/AltR2/-3′

The accompanying tracrRNA was unmodified.

Preparing the AuNP/CRISPR Nanoformulation. crRNAs with 18 spacer-thiol modifications were used. AuNPs in 10 μg/mL concentration was added to crRNA solution in AuNP/crRNA w/w ratio of 0.5. Following that, citrate buffer with the pH of 3 was added in 10 mM concentration and mixed for 5 min. Prepared AuNP/crRNA nanoconjugates were centrifuged down and re-dispersed in PBS. Then, Cpf1 nuclease was added in AuNP/Cpf1 w/w ratio of 0.6. Polyethylenimine (PEI) of 2000 MW was added in 0.005% concentration and mixed thoroughly. In the final step, ssDNA template was added in the AuNP/ssDNA w/w ratio of 1.

Prophetic Example 1. The translational relevance of the pigtail macaque (M. nemestrina) model in terms of cross-reactivity of human reagents and scale has been previously established. This model has served as an important step in clinical translation of HSPC gene therapy. For example, this model has been used to critically evaluate the hematologic potential and safety of LV gene modified and CCR5 edited HSPC, and to track the fate and persistence of these cells in vivo over years. Optimal crRNA pairs identified as above are paired with an optimal nanoparticle delivery strategy identified as above to gene modify autologous HSPC from three nonhuman primates for autologous transplantation after myeloablative, total body irradiation. Safety and feasibility is assessed by measuring product fitness in vitro and in vivo.

In more detail, autologous transplant is performed for CD34⁺CD90⁺CD45RA⁻ HSPC after nanoparticle-mediated CRISPR/Cpf1 GSH editing and reporter transgene insertion. Three juvenile pigtail macaques are primed with recombinant human granulocyte colony stimulating factor (GCS-F; 100 mcg/kg/day×4 days) and stem cell factor (SCF; 50 mcg/kg/day×4 days) as subcutaneous injections. Bone marrow is harvested into heparin on the 4^{th} day of priming. The leukocytes are isolated by ammonium chloride lysis and are labeled with IgM monoclonal antibody 12-8 (CD34⁺) at 4° C. for 30 minutes, washed, and incubated with rat monoclonal anti-mouse IgM microbeads for 30 minutes at 4° C., washed, and then immunoselected. All additional selection procedures will be conducted as described above. The optimal nanoparticle formulation and treatment protocol identified as above is used to accomplish GSH gene editing and targeted GFP transgene insertion. During ex vivo HSPC manipulation monkeys receive myeloablative total body irradiation (1020 cGy) as four fractionated doses over 2 days from a linear accelerator. Gene modified HSPC are formulated in PlasmaLyte and autologous serum for intravenous re-infusion 24 hours after the last irradiation dose is delivered. Animals receive supportive care as needed (transfusions, intravenous fluids, etc.).

Gene modified HSPC products will be tested for viability, in vitro colony-forming capacity (CFC), sterility, Mycoplasma, endotoxin, cell phenotype, indel formation and transgene expression. All test protocols to be applied are approved for use in phase I clinical trials of autologous cell therapy. Safety is determined by the ability to generate autologous nanoparticle-treated cells which are considered suitable for infusion (i.e., sterile, Mycoplasma-free, low endotoxin, >70% viable). Feasibility is determined by the viable cell yield throughout the process and the success of GSH editing and reporter transgene insertion at scale. The target cell dose at infusion is ≥125,000 CD34⁺CD90⁺CD45RA⁻ cells per kilogram of body weight based on our preliminary data.

Safety in vivo is measured by hematologic recovery kinetics and supportive care needs after transplant, as well as clonality of engrafted gene edited cells. Peripheral blood is collected daily from each transplanted animal and analyzed by an automated hematology analyzer until hematologic recovery is observed. Recovery is defined as an absolute neutrophil count >500/mcL and platelets >50,000/mcL for 3 consecutive measurements with an increasing trend observed in both parameters. After hematologic recovery, peripheral blood is collected at least twice per month. Flow cytometry is performed regularly to monitor engraftment levels of nanoparticle-treated cells which express reporter transgene (GFP), as well as lineage markers to determine the extent of multi-lineage reconstitution. Genomic DNA (gDNA) is extracted from these samples and subjected to analysis by Surveyor and DNA barcode and GSH locus sequencing as described above. For DNA barcode sequencing, a single round of PCR amplification is performed and resulting reactions are submitted for sequencing using the Illumina MiSeq platform. Sequence reads are subjected to bioinformatics processing to identify unique barcode events and relative clonal contributions as a measure of clone abundance. Clones are mapped as a function of time and contribution. For GSH locus sequencing the primers corresponding to the optimal crRNA pair identified above are used to sequence the GSH locus. The frequency of engrafted cells containing indels versus reporter transgene cassettes is determined.

This study establishes the scaled protocol for nanoparticle-mediated GSH editing and targeted reporter gene insertion. This protocol serves as the basis for evaluating clinically therapeutic transgene delivery across many disease targets. The results of this study provide the basis for evaluation of strategies for improving gene edited HSPC engraftment and differentiation in this clinically relevant and translational large animal model.

Prophetic Example 2. Validate optimal GSH locus for gene editing in CD34⁺CD45RA⁻ CD90⁺ cells. Healthy donor bone marrow (BM) aspirates are obtained from a commercial provider or under an IRB Protocol. Mobilized peripheral blood products (mAPH) are obtained from healthy donors post granulocyte-colony stimulating factor (G-CSF) administration and leukapheresis collection. For BM, red blood cells are depleted by hetastarch sedimentation, and CD34⁺ cells are immune-selected. For mAPH a standard immunoselection of CD34⁺ cells is performed. Resulting CD34-enriched products from both HSPC sources are stained with antibodies specific to CD90 and CD45RA and the target HSPC population (CD90⁺CD45RA⁻) is collected by fluorescence-activated cell sorting as previously described [Radtke et al., Sci. Transl. Med. 2017; 9 (414).].

CD34+CD45RA−CD90+ cells are targeted with multiple CRISPR/Cpf1 nucleases designed for a candidate GSH identified. Specificity and function is evaluated in vitro and in vivo to identify the optimal GSH locus. Chemically modified mRNA is used to increase intracellular stability, while homology arms and phosphorothioate modification is incorporated into the ssODN to improve HDR efficiency.

Cells are suspended in StemSpan media containing recombinant human growth factors stem cell factor (SCF), thrombopoietin (TPO) and fms-like tyrosine kinase 3 ligand (flt3-L) and incubated at 30° C. for 24 hours. Media is changed the following day and cultures will be prepared for subsequent analysis and infusion. Viability is monitored by flow cytometry and/or trypan blue staining.

At least three methods can be applied to determine the efficiency and specificity of gene addition. First, flow cytometry can be used to assess GFP and/or therapeutic gene expression. This shows the frequency of cells with cassette incorporation and GFP/therapeutic gene expression/function. Second, the Surveyor assay (Ran et al. Cell. 2013; 154(6): 1380-1389) is used to determine the frequency of indels versus transgene insertion at the target locus. Briefly, cells are pelleted and DNA extracted and purified. Resulting DNA is quantified and subjected to PCR using primers designed to the locus targeted by the specific CRISPR/Cpf1 nuclease pair. Appropriate mismatch-containing controls are PCR amplified. Following PCR, products are denatured and re-annealed to form heteroduplexes, which are subsequently treated with Surveyor enzyme and analyzed by gel electrophoresis. Imaging and densitometry are then used to calculate an editing efficiency for each locus. Finally, BLISS is applied to identify off-target DSBs (Yan et al. Nat Commun 2017; 8: 15058). Cells are first transferred to glass slides by cytospinning, followed by permeabilization and fixation. In situ DSBs are polished and ligated to synthetic oligonucleotides containing a unique sample barcode and multiplex identifier, followed by an RA5 Illumina sequencing adaptor and T7 promoter. DNA is then purified and subjected to sonication followed by in vitro transcription and prepared as a library for Illumina-based sequencing. This permits identification of genomic locations of off-target DSBs and quantitation of DSB frequency as the number of reads corresponding to a specific genomic location for a given number of cells immobilized onto the original slide. Following these analyses, the optimal CRISPR/Cpf1 pair and ssODN combination is identified for further analysis in vivo, for example as described below. The optimal combination demonstrates the highest efficiency of barcoded transgene insertion at the desired locus with minimal toxicity in vitro and the lowest frequency of off-target DSB formation across donors and HSPC sources (BM and mAPH).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in gene editing at a targeted GSH site.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Particular embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1-41. (canceled)

42. A gold nanoparticle (AuNP) comprising a gold core associated with at least two active layers,
wherein the AuNP first layer comprises:
a crRNA with a 3′ end and a 5′ end,
wherein the 3′ end is conjugated to a spacer with a thiol modification, and the 5′ end is conjugated to a Cpf1 nuclease to form a crRNA-Cpf1 nuclease ribonucleoprotein (RNP) complex,
wherein the thiol modification is covalently linked to the surface of a gold core of the AuNP, and
wherein the crRNA-Cpf1 nuclease RNP complex binds a target site within human (h) chromosome (chr) 11 or a hchr3 and results in cutting of the DNA at a PAM sequence within the target site; and
wherein the AuNP second layer comprises:
a donor template comprising a therapeutic gene and homology-directed repair templates,
wherein the second AuNP layer is farther from the surface of the gold core than the first AuNP layer.

43. The AuNP of claim 42, wherein:
the target site is hchr11: 67723825-67723849 and has the sequence as set forth in SEQ ID NO: 132;
the target site is hchr11:67691162-67691186 and has the sequence as set forth in SEQ ID NO: 108;
the target site is hchr11:67812349-67812375 and has the sequence as set forth in SEQ ID NO: 203;
the target site is hchr11:67839126-67839150 and has the sequence as set forth in SEQ ID NO: 210; or
the target site is hchr3:46373915-46373939 and has the sequence as set forth in SEQ ID NO: 212.

44. The AuNP of claim 42, wherein the crRNA comprises:
SEQ ID NO: 195 or a sequence with at least 98% identity to SEQ ID NO: 195 that binds within hchr11: 67723825-67723849;
SEQ ID NO: 196 or a sequence with at least 98% identity to SEQ ID NO: 196 that binds within hchr11: 67691162-67691186;
SEQ ID NO: 209 or a sequence with at least 98% identity to SEQ ID NO: 209 that binds within hchr11: 67812349-67812375;
SEQ ID NO: 211 or a sequence with at least 98% identity to SEQ ID NO: 211 that binds within hchr11:67839126-67839150; or
SEQ ID NO: 260 or a sequence with at least 98% identity to SEQ ID NO: 260 that binds within hchr3:46373915-46373939.

45. The AuNP of claim 42, wherein the Cpf1 nuclease has a sequence as set forth in SEQ ID NOs: 229-241 or is encoded by a sequence as set forth in SEQ ID NOs: 216-227.

46. The AuNP of claim 42, wherein the AuNP is coupled to a targeting molecule through a linkage with the Cpf1 nuclease.

47. The AuNP of claim 46, wherein the targeting molecule comprises a CD34 binding domain or a CD90 binding domain.

48. The AuNP of claim 42, wherein the therapeutic gene comprises or encodes skeletal protein 4.1, glycophorin, p55, the Duffy allele, globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C90RF72, α2β1; αvβ3; αvβ5; αvβ63; BOB/GPR15; Bonzo/STRL-33/TYMSTR; CCR2; CCR3; CCRS; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC, laminin receptor, 101F6, 123F2, 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FancA, FancB, FancC, FancD1, FancD2, FancE, FancF, FancG, Fancl, FancJ, FancL, FancM, FancN, FancO, FancP, FancQ, FancR, FancS, FancT, FancU, FancV, and FancW, FCC, FGF, FGR, FHIT, fms, FOX, FUS 1, FUS1, FYN, G-CSF, GDAIF, Gene 21, Gene 26, GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon α, interferon β, interferon γ, IRF-1, JUN, KRAS, LCK, LUCA-1, LUCA-2, LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NTS, OVCA1, p16, p21, p27, p53, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, zac1, iduronidase, IDS, GNS, HGSNAT, SGSH, NAGLU, GUSB, GALNS, GLB1, ARSB, HYAL1, F8, F9, HBB, CYB5R3, 7C, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1.

49. The AuNP of claim 42, wherein the gold core is less than 20 nm in diameter.

50. The AuNP of claim 42, further comprising a positively-charged polymer coating.

51. The AuNP of claim 50, wherein the positively-charged polymer coating surrounds the RNP complex and contacts the surface of the metallic core.

52. The AuNP of claim 50, wherein the positively-charged polymer has a molecular weight of less than 2500 daltons.

53. The AuNP of claim 50, wherein the positively-charged polymer coating comprises polyethyleneimine with a molecular weight of 2000 daltons.

54. A method of providing a therapeutic gene to a patient in need thereof comprising administering a therapeutic formulation to the patient,
wherein the therapeutic formulation comprises a blood cell genetically modified by a gold nanoparticle (AuNP) comprising a gold core associated with a first AuNP layer and a second AuNP layer,
wherein the first NP layer comprises:
a crRNA with a 3′ end and a 5′ end,
wherein the 3′ end is conjugated to a spacer with a thiol modification, and the 5′ end is conjugated to a Cpf1 nuclease to form a crRNA-Cpf1 nuclease ribonucleoprotein (RNP) complex,
wherein the thiol modification is covalently linked to a surface of the gold AuNP core, and
wherein the RNP complex binds a target PAM-site within human (h) chromosome (chr) 11 at positions 67681215-67741765, 67691162-67691186, 67723825-67723849,67805337-67845629, 67812349-67812375, 67812443-67812469, 67839126-67839150, or 67895738-67941098 or hchr3 at positions 46373915-46373939; and
wherein the second AuNP layer comprises:
a donor template comprising a therapeutic gene and homology-directed repair templates; and
wherein the second AuNP layer is farther from the surface of the metallic AuNP core than the first NP layer,
thereby providing the therapeutic gene to the patient.

55. The method of claim 54, wherein the therapeutic formulation further comprises serum components.

56. The method of claim 54, wherein the patient in need thereof has been diagnosed with a monogenetic blood disorder, hemophilia, Grave's Disease, rheumatoid arthritis, pernicious anemia, Multiple Sclerosis (MS), inflammatory bowel disease, systemic lupus erythematosus (SLE), Wiskott-Aldrich syndrome (WAS), chronic granulomatous disease (CGD), Battens disease, adrenoleukodystrophy (ALD) or metachromatic leukodystrophy (MLD), muscular dystrophy, pulmonary aveolar proteinosis (PAP), pyruvate kinase deficiency, Shwachmann-Diamond-Blackfan anemia, dyskeratosis congenita, cystic fibrosis, Parkinson's disease, Alzheimer's disease, amyotrophic lateral sclerosis (Lou Gehrig's disease), acute lymphoblastic leukemia (ALL), acute myelogenous leukemia (AML), agnogenic myeloid metaplasia, amegakaryocytosis/congenital thrombocytopenia, ataxia telangiectasia, 0-thalassemia major, CLL, chronic myelogenous leukemia (CML), chronic myelomonocytic leukemia, common variable immune deficiency (CVID), complement disorders, congenital (Xlinked) agammaglobulinemia, familial erythrophagocytic lymphohistiocytosis, Hodgkin's lymphoma, Hurler's syndrome, hyper IgM, IgG subclass deficiency, juvenile myelomonocytic leukemia, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, selective IgA deficiency, severe aplastic anemia, SCD, specific antibody deficiency, a bacterial infection, or a parasitic infection.

57. The method of claim 54, wherein the therapeutic formulation is administered by injection, infusion, perfusion, or lavage.

58. The method of claim 54, wherein the blood cell is selected from the group consisting of hematopoietic progenitor cells (HPC), hematopoietic stem and progenitor cell (HSPCs), T cells, natural killer (NK) cells, B cells, macrophages, monocytes, mesenchymal stem cells (MSC), white blood cell (WBC), mononuclear cell (MNC), endothelial cells (EC), stromal cells, and bone marrow fibroblasts.


SEQUENCE LISTING (264 sequences)

SEQ ID NO: 1 - DNA, 25 bp, Homo sapiens
  source 1..25: mol_type = genomic DNA
tttaaaaaaagtcttcataataaaa

SEQ ID NO: 2 - DNA, 25 bp, Homo sapiens
  source 1..25: mol_type = genomic DNA
tttacttctgtgcacccatagactc

SEQ ID NO: 3 - DNA, 25 bp, Homo sapiens
  source 1..25: mol_type = genomic DNA
tttaactctgccatgggtgccagga

SEQ ID NO: 4 - DNA, 25 bp, Homo sapiens
  source 1..25: mol_type = genomic DNA
tttagccactgtgagaaacagttta

SEQ ID NO: 5 - DNA, 25 bp, Homo sapiens
  source 1..25: mol_type = genomic DNA
tttataaggctaagtagtattctat

SEQ ID NO: 6 - DNA, 25 bp, Homo sapiens
  source 1..25: mol_type = genomic DNA
tttatccactaattgttgatgggca

SEQ ID NO: 7 - DNA, 25 bp, Homo sapiens
  source 1..25: mol_type = genomic DNA
tttattttctttttggtaagaagga

SEQ ID NO: 8 - DNA, 25 bp, Homo sapiens
  source 1..25: mol_type = genomic DNA
tttaaaataatcgtcattctttttg

SEQ ID NO: 9 - DNA, 25 bp, Homo sapiens
  source 1..25: mol_type = genomic DNA
tttatattaatccttgtatgtctga

SEQ ID NO: 10 - DNA, 25 bp, Homo sapiens
  source 1..25: mol_type = genomic DNA
tttaaatggataagttaggctgggc

SEQ ID NO: 11 - DNA, 25 bp, Homo sapiens
  source 1..25: mol_type = genomic DNA
tttacacaaactgtacttgtaggta

SEQ ID NO: 12 - DNA, 25 bp, Homo sapiens
  source 1..25: mol_type = genomic DNA
tttagtgaggaaaaactacatttat

SEQ ID NO: 13 - DNA, 25 bp, Homo sapiens
  source 1..25: mol_type = genomic DNA
tttaaaaagggaaaattagggagaa

SEQ ID NO: 14 - DNA, 25 bp, Homo sapiens
  source 1..25: mol_type = genomic DNA
tttactaaatgacctcttcgccaaa

SEQ ID NO: 15 - DNA, 25 bp, Homo sapiens
  source 1..25: mol_type = genomic DNA
tttagtgatatgaaaagaaccatgc

SEQ ID NO: 16 - DNA, 25 bp, Homo sapiens
  source 1..25: mol_type = genomic DNA
tttaaattttccagtgtctcagtgt

SEQ ID NO: 17 - DNA, 25 bp, Homo sapiens
  source 1..25: mol_type = genomic DNA
tttaagcctgaaaacctaaaaaatg

SEQ ID NO: 18 - DNA, 25 bp, Homo sapiens
  source 1..25: mol_type = genomic DNA
tttaaaaattagctgcgttggtggt

SEQ ID NO: 19 - DNA, 25 bp, Homo sapiens
  source 1..25: mol_type = genomic DNA
tttagctgctttaaaatgtgaactc

SEQ ID NO: 20 - DNA, 25 bp, Homo sapiens
  source 1..25: mol_type = genomic DNA
tttaatctcaagtcacttctctagg

SEQ ID NO: 21 - DNA, 25 bp, Homo sapiens
  source 1..25: mol_type = genomic DNA
tttaaacgctcgttagatcactgga

SEQ ID NO: 22 - DNA, 25 bp, Homo sapiens
  source 1..25: mol_type = genomic DNA
tttaaggcaggaatcagggtgccat

SEQ ID NO: 23 - DNA, 25 bp, Homo sapiens
  source 1..25: mol_type = genomic DNA
tttaaaaaaatcccaggtgacccta

SEQ ID NO: 24 - DNA, 25 bp, Homo sapiens
  source 1..25: mol_type = genomic DNA
tttaatcttgcagggacaggaagga

SEQ ID NO: 25 - DNA, 25 bp, Homo sapiens
  source 1..25: mol_type = genomic DNA
tttaaaatgtgaactccaaatcagg

SEQ ID NO: 26 - DNA, 25 bp, Homo sapiens
  source 1..25: mol_type = genomic DNA
tttattatttcgttgtttttcctct

SEQ ID NO: 27 - DNA, 25 bp, Homo sapiens
  source 1..25: mol_type = genomic DNA
tttatcttggtgtttgaactcggat

SEQ ID NO: 28 - DNA, 25 bp, Homo sapiens
  source 1..25: mol_type = genomic DNA
tttactgcccatttagctgctttaa

SEQ ID NO: 29 - DNA, 25 bp, Homo sapiens
  source 1..25: mol_type = genomic DNA
tttaacttgattaccttatagtcaa

SEQ ID NO: 30 - DNA, 25 bp, Homo sapiens
  source 1..25: mol_type = genomic DNA
tttatatatttaacttctgtatttt

SEQ ID NO: 31 - DNA, 25 bp, Homo sapiens
  source 1..25: mol_type = genomic DNA
tttatctgctatagctaacaaattt

SEQ ID NO: 32 - DNA, 25 bp, Homo sapiens
  source 1..25: mol_type = genomic DNA
tttaacttctgtattttctaaaact

SEQ ID NO: 33 - DNA, 25 bp, Homo sapiens
  source 1..25: mol_type = genomic DNA
tttataacacaccagcatcagttac

SEQ ID NO: 34 - DNA, 25 bp, Homo sapiens
  source 1..25: mol_type = genomic DNA
tttattatctttcctgttttctaat

SEQ ID NO: 35 - DNA, 25 bp, Homo sapiens
  source 1..25: mol_type = genomic DNA
tttaatattaacatttgctaatttc

SEQ ID NO: 36 - DNA, 25 bp, Homo sapiens
  source 1..25: mol_type = genomic DNA
tttaaaaggccagatgtcaattcag

SEQ ID NO: 37 - DNA, 25 bp, Homo sapiens
  source 1..25: mol_type = genomic DNA
tttatgcagatagatatatattttt

SEQ ID NO: 38 - DNA, 25 bp, Homo sapiens
  source 1..25: mol_type = genomic DNA
tttaatcactggatcatggacctaa

SEQ ID NO: 39 - DNA, 25 bp, Homo sapiens
  source 1..25: mol_type = genomic DNA
tttagtgtttcttagaaacttaaca

SEQ ID NO: 40 - DNA, 25 bp, Homo sapiens
  source 1..25: mol_type = genomic DNA
tttacagcagtgccattcacaatgg

SEQ ID NO: 41 - DNA, 25 bp, Homo sapiens
  source 1..25: mol_type = genomic DNA
tttataggcccaggtctagatctgg

SEQ ID NO: 42 - DNA, 25 bp, Homo sapiens
  source 1..25: mol_type = genomic DNA
tttagccctatcttatccatatgga

SEQ ID NO: 43 - DNA, 25 bp, Homo sapiens
  source 1..25: mol_type = genomic DNA
tttatgtctagtacttagggcagta

SEQ ID NO: 44 - DNA, 25 bp, Homo sapiens
  source 1..25: mol_type = genomic DNA
tttacaattaattgtagttctttga

SEQ ID NO: 45 - DNA, 25 bp, Homo sapiens
  source 1..25: mol_type = genomic DNA
tttattttcttagatttactctgta

SEQ ID NO: 46 - DNA, 25 bp, Homo sapiens
  source 1..25: mol_type = genomic DNA
tttaaaaactgggtttacaaataaa

SEQ ID NO: 47 - DNA, 25 bp, Homo sapiens
  source 1..25: mol_type = genomic DNA
tttaaacattttgactgtagccatt

SEQ ID NO: 48 - DNA, 25 bp, Homo sapiens
  source 1..25: mol_type = genomic DNA
tttatgactgtttcatgtgtgctca

SEQ ID NO: 49 - DNA, 25 bp, Homo sapiens
  source 1..25: mol_type = genomic DNA
tttataacctcacctttggctttta

SEQ ID NO: 50 - DNA, 25 bp, Homo sapiens
  source 1..25: mol_type = genomic DNA
tttagaagtccatataaggggatgc

SEQ ID NO: 51 - DNA, 25 bp, Homo sapiens
  source 1..25: mol_type = genomic DNA
tttactgttaattagtccttgctta

SEQ ID NO: 52 - DNA, 25 bp, Homo sapiens
  source 1..25: mol_type = genomic DNA
tttatctatgaacctcataggtcct

SEQ ID NO: 53 - DNA, 25 bp, Homo sapiens
  source 1..25: mol_type = genomic DNA
tttactatagttaattggaacactt

SEQ ID NO: 54 - DNA, 25 bp, Homo sapiens
  source 1..25: mol_type = genomic DNA
tttaacgttaaatctctttctaaca

SEQ ID NO: 55 - DNA, 25 bp, Homo sapiens
  source 1..25: mol_type = genomic DNA
tttatctcctttaaattcccatgtt

SEQ ID NO: 56 - DNA, 25 bp, Homo sapiens
  source 1..25: mol_type = genomic DNA
tttagtccaaataaagcaacaatac

SEQ ID NO: 57 - DNA, 25 bp, Homo sapiens
  source 1..25: mol_type = genomic DNA
tttatccattcccaaccacaaagaa

SEQ ID NO: 58 - DNA, 25 bp, Homo sapiens
  source 1..25: mol_type = genomic DNA
tttaatttctccacttgattaactt

SEQ ID NO: 59 - DNA, 25 bp, Homo sapiens
  source 1..25: mol_type = genomic DNA
tttagccaaaggacatgcctaaaat

SEQ ID NO: 60 - DNA, 25 bp, Homo sapiens
  source 1..25: mol_type = genomic DNA
tttaggaattaataaaaattgtata

SEQ ID NO: 61 - DNA, 25 bp, Homo sapiens
  source 1..25: mol_type = genomic DNA
tttaatgcatccataaacagaactg

SEQ ID NO: 62 - DNA, 25 bp, Homo sapiens
  source 1..25: mol_type = genomic DNA
tttagggtggttctcctgggatttt

SEQ ID NO: 63 - DNA, 25 bp, Homo sapiens
  source 1..25: mol_type = genomic DNA
tttatcccttacgcatgaggtccct

SEQ ID NO: 64 - DNA, 25 bp, Homo sapiens
  source 1..25: mol_type = genomic DNA
tttacacaagcaacaccagctgcag

SEQ ID NO: 65 - DNA, 25 bp, Homo sapiens
  source 1..25: mol_type = genomic DNA
tttaaatacttgacaaaaaagattg

SEQ ID NO: 66 - DNA, 25 bp, Homo sapiens
  source 1..25: mol_type = genomic DNA
tttaaaaggggtctctactaaatct

SEQ ID NO: 67 - DNA, 25 bp, Homo sapiens
  source 1..25: mol_type = genomic DNA
tttaatctttatctgacctaaattt

SEQ ID NO: 68 - DNA, 25 bp, Homo sapiens
  source 1..25: mol_type = genomic DNA
tttataaagaaattccgcaagaact

SEQ ID NO: 69 - DNA, 25 bp, Homo sapiens
  source 1..25: mol_type = genomic DNA
tttatgatttaaaggggaagctttg

SEQ ID NO: 70 - DNA, 25 bp, Homo sapiens
  source 1..25: mol_type = genomic DNA
tttaatgctttcaacatcgattgct

SEQ ID NO: 71 - DNA, 25 bp, Homo sapiens
  source 1..25: mol_type = genomic DNA
tttacggagaggatacaaagatcct

SEQ ID NO: 72 - DNA, 25 bp, Homo sapiens
  source 1..25: mol_type = genomic DNA
tttaagatcttcctataattatagc

SEQ ID NO: 73 - DNA, 25 bp, Homo sapiens
  source 1..25: mol_type = genomic DNA
tttaatttttgttggagtcttttct

SEQ ID NO: 74 - DNA, 25 bp, Homo sapiens
  source 1..25: mol_type = genomic DNA
tttattttttaaaaccagaacattt

SEQ ID NO: 75 - DNA, 25 bp, Homo sapiens
  source 1..25: mol_type = genomic DNA
tttagttctctttttatactccaaa

SEQ ID NO: 76 - DNA, 25 bp, Homo sapiens
  source 1..25: mol_type = genomic DNA
tttactaagaatagtgtaggggtta

SEQ ID NO: 77 - DNA, 25 bp, Homo sapiens
  source 1..25: mol_type = genomic DNA
tttactatttcccatcttatgtata

SEQ ID NO: 78 - DNA, 25 bp, Homo sapiens
  source 1..25: mol_type = genomic DNA
tttagcaggtgtcttgatccccctt

SEQ ID NO: 79 - DNA, 25 bp, Homo sapiens
  source 1..25: mol_type = genomic DNA
tttattttctagtccccctttgatc

SEQ ID NO: 80 - DNA, 25 bp, Homo sapiens
  source 1..25: mol_type = genomic DNA
tttaaaatcatcttattgtttacaa

SEQ ID NO: 81 - DNA, 25 bp, Homo sapiens
  source 1..25: mol_type = genomic DNA
tttagagagatatattttcctctag

SEQ ID NO: 82 - DNA, 25 bp, Homo sapiens
  source 1..25: mol_type = genomic DNA
tttaaaagtggtcacaagtggggga

SEQ ID NO: 83 - DNA, 25 bp, Homo sapiens
  source 1..25: mol_type = genomic DNA
tttactagaaacctttcccatattg

SEQ ID NO: 84 - DNA, 25 bp, Homo sapiens
  source 1..25: mol_type = genomic DNA
tttattgctctgtaacagattacca

SEQ ID NO: 85 - DNA, 25 bp, Homo sapiens
  source 1..25: mol_type = genomic DNA
tttaccagccatcttagaacaaatt

SEQ ID NO: 86 - DNA, 25 bp, Homo sapiens
  source 1..25: mol_type = genomic DNA
tttacagaattcgctttccctttaa

SEQ ID NO: 87 - DNA, 25 bp, Homo sapiens
  source 1..25: mol_type = genomic DNA
tttattaagctaaacctaggtacaa

SEQ ID NO: 88 - DNA, 25 bp, Homo sapiens
  source 1..25: mol_type = genomic DNA
tttaggatcaccttaacttggtgag

SEQ ID NO: 89 - DNA, 25 bp, Homo sapiens
  source 1..25: mol_type = genomic DNA
tttactatttcccctggagtcttta

SEQ ID NO: 90 - DNA, 25 bp, Homo sapiens
  source 1..25: mol_type = genomic DNA
tttaataagtcttttgattacaggc

SEQ ID NO: 91 - DNA, 25 bp, Homo sapiens
  source 1..25: mol_type = genomic DNA
tttactctgtagctttttaaaatca

SEQ ID NO: 92 - DNA, 25 bp, Homo sapiens
  source 1..25: mol_type = genomic DNA
tttacttcttttaagatcttcctat

SEQ ID NO: 93 - DNA, 25 bp, Homo sapiens
  source 1..25: mol_type = genomic DNA
tttactttgctctgtgaacagagtt

SEQ ID NO: 94 - DNA, 25 bp, Homo sapiens
  source 1..25: mol_type = genomic DNA
tttaattcctgtttcattttcccat

SEQ ID NO: 95 - DNA, 25 bp, Homo sapiens
  source 1..25: mol_type = genomic DNA
tttagggcgtgactgtgaataactc

SEQ ID NO: 96 - DNA, 25 bp, Homo sapiens
  source 1..25: mol_type = genomic DNA
tttattcaatttctcctaagtctgc

SEQ ID NO: 97 - DNA, 25 bp, Homo sapiens
  source 1..25: mol_type = genomic DNA
tttaaaaatatttagcaactgggac

SEQ ID NO: 98 - DNA, 25 bp, Homo sapiens
  source 1..25: mol_type = genomic DNA
tttacgttcccagatcgtatttctt

SEQ ID NO: 99 - DNA, 25 bp, Homo sapiens
  source 1..25: mol_type = genomic DNA
tttagttcatggcaagcaagtcatt

SEQ ID NO: 100 - DNA, 25 bp, Homo sapiens
  source 1..25: mol_type = genomic DNA
tttaggccaccaattgggggcattt

SEQ ID NO: 101 - DNA, 25 bp, Homo sapiens
  source 1..25: mol_type = genomic DNA
tttaccaaccatcactgccatcgtc

SEQ ID NO: 102 - DNA, 25 bp, Homo sapiens
  source 1..25: mol_type = genomic DNA
tttaaccccagaaactgttaattcc

SEQ ID NO: 103 - DNA, 25 bp, Homo sapiens
  source 1..25: mol_type = genomic DNA
tttatagttatttacagaattcgct

SEQ ID NO: 104 - DNA, 25 bp, Homo sapiens
  source 1..25: mol_type = genomic DNA
tttatttgtgcaacaatggggaatt

SEQ ID NO: 105 - DNA, 25 bp, Homo sapiens
  source 1..25: mol_type = genomic DNA
tttaataaagcaataggaagacgtt

SEQ ID NO: 106 - DNA, 25 bp, Homo sapiens
  source 1..25: mol_type = genomic DNA
tttaagtcacaggggtgtagaccct

SEQ ID NO: 107 - DNA, 25 bp, Homo sapiens
  source 1..25: mol_type = genomic DNA
tttagtgaccaccctactctattgt

SEQ ID NO: 108 - DNA, 25 bp, Homo sapiens
  source 1..25: mol_type = genomic DNA
tttaatagaatagcctcatatttta

SEQ ID NO: 109 - DNA, 25 bp, Homo sapiens
  source 1..25: mol_type = genomic DNA
tttaaccactcccactcccaattac

SEQ ID NO: 110 - DNA, 25 bp, Homo sapiens
  source 1..25: mol_type = genomic DNA
tttagatggaactagcattccacaa

SEQ ID NO: 111 - DNA, 25 bp, Homo sapiens
  source 1..25: mol_type = genomic DNA
tttaaaagtagcagcttaagccaga

SEQ ID NO: 112 - DNA, 25 bp, Homo sapiens
  source 1..25: mol_type = genomic DNA
tttagttaatttcttatataagagc

SEQ ID NO: 113 - DNA, 25 bp, Homo sapiens
  source 1..25: mol_type = genomic DNA
tttaaggtagatctgtgcaggggga

SEQ ID NO: 114 - DNA, 25 bp, Homo sapiens
  source 1..25: mol_type = genomic DNA
tttactcctcccgaagaggatggat

SEQ ID NO: 115 - DNA, 25 bp, Homo sapiens
  source 1..25: mol_type = genomic DNA
tttaacatagatattgaagtcagag

SEQ ID NO: 116 - DNA, 25 bp, Homo sapiens
  source 1..25: mol_type = genomic DNA
tttatcatattactattttgccagt

SEQ ID NO: 117 - DNA, 25 bp, Homo sapiens
  source 1..25: mol_type = genomic DNA
tttagttacatgatttttaagagtt

SEQ ID NO: 118 - DNA, 25 bp, Homo sapiens
  source 1..25: mol_type = genomic DNA
tttacctggttctgtaaatatttta

SEQ ID NO: 119 - DNA, 25 bp, Homo sapiens
  source 1..25: mol_type = genomic DNA
tttattatgtgagtgataaatttga

SEQ ID NO: 120 - DNA, 25 bp, Homo sapiens
  source 1..25: mol_type = genomic DNA
tttacaccccccacccccgaggcct

SEQ ID NO: 121 - DNA, 25 bp, Homo sapiens
  source 1..25: mol_type = genomic DNA
tttaactgaacatgtgttggaggaa

SEQ ID NO: 122 - DNA, 25 bp, Homo sapiens
  source 1..25: mol_type = genomic DNA
tttaatttatctatgaacctcatag

SEQ ID NO: 123 - DNA, 25 bp, Homo sapiens
  source 1..25: mol_type = genomic DNA
tttatttttttattttttaaaacca

SEQ ID NO: 124 - DNA, 25 bp, Homo sapiens
  source 1..25: mol_type = genomic DNA
tttatcctttttactcctcccgaag

SEQ ID NO: 125 - DNA, 25 bp, Homo sapiens
  source 1..25: mol_type = genomic DNA
tttaaacttttttaaataggtaaag

SEQ ID NO: 126 - DNA, 25 bp, Homo sapiens
  source 1..25: mol_type = genomic DNA
tttaatttttaataaagcaatagga

SEQ ID NO: 127 - DNA, 25 bp, Homo sapiens
  source 1..25: mol_type = genomic DNA
tttacatatagtttttgagcctttt

SEQ ID NO: 128 - DNA, 25 bp, Homo sapiens
  source 1..25: mol_type = genomic DNA
tttaaaagttttaccagccatctta

SEQ ID NO: 129 - DNA, 25 bp, Homo sapiens
  source 1..25: mol_type = genomic DNA
tttatttgggctatttgccaaacag

SEQ ID NO: 130 - DNA, 25 bp, Homo sapiens
  source 1..25: mol_type = genomic DNA
tttaactatggttcctttaaatcag

SEQ ID NO: 131 - DNA, 25 bp, Homo sapiens
  source 1..25: mol_type = genomic DNA
tttataaggggacaatccaacatct

SEQ ID NO: 132 - DNA, 25 bp, Homo sapiens
  source 1..25: mol_type = genomic DNA
tttattcggacccgtgctacaactt

SEQ ID NO: 133 - DNA, 25 bp, Homo sapiens
  source 1..25: mol_type = genomic DNA
tttattatccattttaacttgatta

SEQ ID NO: 134 - DNA, 25 bp, Homo sapiens
  source 1..25: mol_type = genomic DNA
tttatcagttgtccaatttgtggtg

SEQ ID NO: 135 - DNA, 25 bp, Homo sapiens
  source 1..25: mol_type = genomic DNA
tttaaattcccatgttgcaacccta

SEQ ID NO: 136 - DNA, 25 bp, Homo sapiens
  source 1..25: mol_type = genomic DNA
tttaataatttttctacttatactt

SEQ ID NO: 137 - DNA, 25 bp, Homo sapiens
  source 1..25: mol_type = genomic DNA
tttacccagtgggtaaaatgatcta

SEQ ID NO: 138 - DNA, 25 bp, Homo sapiens
  source 1..25: mol_type = genomic DNA
tttaggtgtatgatacttttagtgc

SEQ ID NO: 139 - DNA, 25 bp, Homo sapiens
  source 1..25: mol_type = genomic DNA
tttaggttctacatattgaagcttt

SEQ ID NO: 140 - DNA, 25 bp, Homo sapiens
  source 1..25: mol_type = genomic DNA
tttaagttcttgtttggttcggggc

SEQ ID NO: 141 - DNA, 25 bp, Homo sapiens
  source 1..25: mol_type = genomic DNA
tttattcatcacaacaggtaagtcc

SEQ ID NO: 142 - DNA, 25 bp, Homo sapiens
  source 1..25: mol_type = genomic DNA
tttaaaggataaagaataatatagg

SEQ ID NO: 143 - DNA, 25 bp, Homo sapiens
  source 1..25: mol_type = genomic DNA
tttattttcacatccacagctccta

SEQ ID NO: 144 - DNA, 25 bp, Homo sapiens
  source 1..25: mol_type = genomic DNA
tttagtccccgcatcgtgtgggggg

SEQ ID NO: 145 - DNA, 25 bp, Homo sapiens
  source 1..25: mol_type = genomic DNA
tttatttagatggaactagcattcc

SEQ ID NO: 146 - DNA, 25 bp, Homo sapiens
  source 1..25: mol_type = genomic DNA
tttatctgcacttattaaatggcct

SEQ ID NO: 147 - DNA, 25 bp, Homo sapiens
  source 1..25: mol_type = genomic DNA
tttatccttggacataattaaagaa

SEQ ID NO: 148 - DNA, 25 bp, Homo sapiens
  source 1..25: mol_type = genomic DNA
tttaacagtggctttagttctcttt

SEQ ID NO: 149 - DNA, 25 bp, Homo sapiens
  source 1..25: mol_type = genomic DNA
tttagtacagcagcctgaactgact

SEQ ID NO: 150 - DNA, 25 bp, Homo sapiens
  source 1..25: mol_type = genomic DNA
tttaaaacgacctggtctcccgcat

SEQ ID NO: 151 - DNA, 25 bp, Homo sapiens
  source 1..25: mol_type = genomic DNA
tttactctgtgacaatatattctat

SEQ ID NO: 152 - DNA, 25 bp, Homo sapiens
  source 1..25: mol_type = genomic DNA
tttatactccaaacttcagacccag

SEQ ID NO: 153 - DNA, 25 bp, Homo sapiens
  source 1..25: mol_type = genomic DNA
tttaaatttagtttttttattatct

SEQ ID NO: 154 - DNA, 25 bp, Homo sapiens
  source 1..25: mol_type = genomic DNA
tttaaataggtaaaggcagggagga

SEQ ID NO: 155 - DNA, 25 bp, Homo sapiens
  source 1..25: mol_type = genomic DNA
tttaactcctctttttctttctgga

SEQ ID NO: 156 - DNA, 25 bp, Homo sapiens
  source 1..25: mol_type = genomic DNA
tttaatttgggaatattgggttaat

SEQ ID NO: 157 - DNA, 25 bp, Homo sapiens
  source 1..25: mol_type = genomic DNA
tttagagtcagtatagatggttttt

SEQ ID NO: 158 - DNA, 25 bp, Homo sapiens
  source 1..25: mol_type = genomic DNA
tttatgctgggaaccggagggctgg

SEQ ID NO: 159 - DNA, 25 bp, Homo sapiens
  source 1..25: mol_type = genomic DNA
tttaaaagaaaagttaggttggtgt

SEQ ID NO: 160 - DNA, 25 bp, Homo sapiens
  source 1..25: mol_type = genomic DNA
tttatgttgtacatgccacaaaaaa

SEQ ID NO: 161 - DNA, 25 bp, Homo sapiens
  source 1..25: mol_type = genomic DNA
tttagtaatgtctggccaactgtga

SEQ ID NO: 162 - DNA, 25 bp, Homo sapiens
  source 1..25: mol_type = genomic DNA
tttatttcttgttgggaggatgagg

SEQ ID NO: 163 - DNA, 25 bp, Homo sapiens
  source 1..25: mol_type = genomic DNA
tttaattaaggctttgactgcatta

SEQ ID NO: 164 - DNA, 25 bp, Homo sapiens
  source 1..25: mol_type = genomic DNA
tttacactcttcactcgctttgtcc

SEQ ID NO: 165 - DNA, 25 bp, Homo sapiens
  source 1..25: mol_type = genomic DNA
tttaagagtttgatttatccttttt

SEQ ID NO: 166 - DNA, 25 bp, Homo sapiens
  source 1..25: mol_type = genomic DNA
tttagctatttgttatggcagcaac

SEQ ID NO: 167 - DNA, 25 bp, Homo sapiens
  source 1..25: mol_type = genomic DNA
tttattatctttccaatactttaac

SEQ ID NO: 168 - DNA, 25 bp, Homo sapiens
  source 1..25: mol_type = genomic DNA
tttaaggcttgtttatttgtgtttt

SEQ ID NO: 169 - DNA, 25 bp, Homo sapiens
  source 1..25: mol_type = genomic DNA
tttatctggtccccgaggcagtgca

SEQ ID NO: 170 - DNA, 25 bp, Homo sapiens
  source 1..25: mol_type = genomic DNA
tttaaagaaggatatttagaatttt

SEQ ID NO: 171 - DNA, 25 bp, Homo sapiens
  source 1..25: mol_type = genomic DNA
tttaaaggtaggcctcaaaaagaac

SEQ ID NO: 172 - DNA, 25 bp, Homo sapiens
  source 1..25: mol_type = genomic DNA
tttatttgttcttttaatttatcta

SEQ ID NO: 173 - DNA, 25 bp, Homo sapiens
  source 1..25: mol_type = genomic DNA
tttatctgacctaaattttgaccaa

SEQ ID NO: 174 - DNA, 25 bp, Homo sapiens
  source 1..25: mol_type = genomic DNA
tttaggctcctgggattcacaagaa

SEQ ID NO: 175 - DNA, 25 bp, Homo sapiens
  source 1..25: mol_type = genomic DNA
tttactggcaaactgggaggagaga

SEQ ID NO: 176 - DNA, 25 bp, Homo sapiens
  source 1..25: mol_type = genomic DNA
tttaaccttaacgtgcttgaggttt

SEQ ID NO: 177 - DNA, 25 bp, Homo sapiens
  source 1..25: mol_type = genomic DNA
tttatttctatattttgaggacatg

SEQ ID NO: 178 - DNA, 25 bp, Homo sapiens
  source 1..25: mol_type = genomic DNA
tttagaatttttaggctcctgggat

SEQ ID NO: 179 - DNA, 25 bp, Homo sapiens
  source 1..25: mol_type = genomic DNA
tttatgatttgctgccagaacattt

SEQ ID NO: 180 - DNA, 25 bp, Homo sapiens
  source 1..25: mol_type = genomic DNA
tttattgattttttaaattttctaa

SEQ ID NO: 181 - DNA, 25 bp, Homo sapiens
  source 1..25: mol_type = genomic DNA
tttatcccactgcgggtcctgagca

SEQ ID NO: 182 - DNA, 25 bp, Homo sapiens
  source 1..25: mol_type = genomic DNA
tttataatttccatgctttttcagt

SEQ ID NO: 183 - DNA, 25 bp, Homo sapiens
  source 1..25: mol_type = genomic DNA
tttatctgtaattctgcagaccctc

SEQ ID NO: 184 - DNA, 25 bp, Homo sapiens
  source 1..25: mol_type = genomic DNA
tttaaaggggaagctttgaagagga

SEQ ID NO: 185 - DNA, 25 bp, Homo sapiens
  source 1..25: mol_type = genomic DNA
tttacctgccggtagtccttggtcc

SEQ ID NO: 186 - DNA, 25 bp, Homo sapiens
  source 1..25: mol_type = genomic DNA
tttaccaatgtgttctaagttttca

SEQ ID NO: 187 - DNA, 25 bp, Homo sapiens
  source 1..25: mol_type = genomic DNA
tttaaaaaaataaatactgaccttg

SEQ ID NO: 188 - DNA, 25 bp, Homo sapiens
  source 1..25: mol_type = genomic DNA
tttatcttgtaggtggttaagaact

SEQ ID NO: 189 - DNA, 25 bp, Homo sapiens
  source 1..25: mol_type = genomic DNA
tttatttcttttcacgaattgctgg

SEQ ID NO: 190 - DNA, 25 bp, Homo sapiens
  source 1..25: mol_type = genomic DNA
tttatgtggtgttcagagccccagg

SEQ ID NO: 191 - DNA, 25 bp, Homo sapiens
  source 1..25: mol_type = genomic DNA
tttatcggtgttattgatgatcatt

SEQ ID NO: 192 - DNA, 25 bp, Homo sapiens
  source 1..25: mol_type = genomic DNA
tttattttatttcttgttgggagga

SEQ ID NO: 193 - DNA, 25 bp, Homo sapiens
  source 1..25: mol_type = genomic DNA
tttaaattggctataaatctttgac

SEQ ID NO: 194 - DNA, 25 bp, Homo sapiens
  source 1..25: mol_type = genomic DNA
tttacctttactgttaattagtcct

SEQ ID NO: 195 - RNA, 41 nt, synthetic construct
  misc_feature 1..41: note = crRNA for SEQ ID NO: 132
  source 1..41: mol_type = other RNA
taatttctactcttgtagatttcggacccgtgctacaactt

SEQ ID NO: 196 - RNA, 41 nt, synthetic construct
  misc_feature 1..41: note = crRNA for Cpf1 target site of SEQ ID NO: 108
  source 1..41: mol_type = other RNA
taatttctactcttgtagatatagaatagcctcatatttta

SEQ ID NO: 197 - DNA, 27 bp, Homo sapiens
  source 1..27: mol_type = genomic DNA
tttgtgtccccgttttggttggtaaac

SEQ ID NO: 198 - DNA, 27 bp, Homo sapiens
  source 1..27: mol_type = genomic DNA
tttaaaaatcaataccgataataatga

SEQ ID NO: 199 - DNA, 27 bp, Homo sapiens
  source 1..27: mol_type = genomic DNA
tttcttaatatgaatattaatatcggt

SEQ ID NO: 200 - DNA, 27 bp, Homo sapiens
  source 1..27: mol_type = genomic DNA
tttccgtatctggaaggggcatcttgg

SEQ ID NO: 201 - DNA, 27 bp, Homo sapiens
  source 1..27: mol_type = genomic DNA
tttccttaggaccggaaggattacagc

SEQ ID NO: 202 - DNA, 27 bp, Homo sapiens
  source 1..27: mol_type = genomic DNA
tttgcctaaaaggcactatgtcaaatg

SEQ ID NO: 203 - DNA, 27 bp, Homo sapiens
  source 1..27: mol_type = genomic DNA
tttggagctgttggcatcatgttcctg

SEQ ID NO: 204 - DNA, 27 bp, Homo sapiens
  source 1..27: mol_type = genomic DNA
tttgattcttttctatctcaggacaga

SEQ ID NO: 205 - DNA, 27 bp, Homo sapiens
  source 1..27: mol_type = genomic DNA
tttatagacatcccacactgtagttct

SEQ ID NO: 206 - DNA, 27 bp, Homo sapiens
  source 1..27: mol_type = genomic DNA
tttattaatttgagaaccaacataagg

SEQ ID NO: 207 - DNA, 27 bp, Homo sapiens
  source 1..27: mol_type = genomic DNA
tttattttctttttggtaagaaggaac

SEQ ID NO: 208 - DNA, 27 bp, Homo sapiens
  source 1..27: mol_type = genomic DNA
tttcacacacacacacacacacacaca

SEQ ID NO: 209 - RNA, 43 nt, synthetic construct
  misc_feature 1..43: note = crRNA for Cpf1 target site of SEQ ID NO: 203
  source 1..43: mol_type = other RNA
taatttctactcttgtagatgagctgttggcatcatgttcctg

SEQ ID NO: 210 - DNA, 25 bp, Homo sapiens
  source 1..25: mol_type = genomic DNA
tttatccaaacctcctaaatgatac

SEQ ID NO: 211 - RNA, 41 nt, synthetic construct
  misc_feature 1..41: note = crRNA for Cpf1 target site of SEQ ID NO: 210
  source 1..41: mol_type = other RNA
taatttctactcttgtagattccaaacctcctaaatgatac

SEQ ID NO: 212 - DNA, 25 bp, Homo sapiens
  source 1..25: mol_type = genomic DNA
tttacacccgatccactggggagca

SEQ ID NO: 213 - DNA, 27 bp, Homo sapiens
  source 1..27: mol_type = genomic DNA
tttttgattcttttctatctcaggaca

SEQ ID NO: 214 - DNA, 511 bp, Homo sapiens
  source 1..511: mol_type = genomic DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 215 - DNA, 4107 bp, Streptococcus pyogenes
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 216 - DNA, 3903 bp, Francisella tularensis
  source 1..3903: mol_type = genomic DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctgaatatataaaggactcagagaaatttaagaatttgtttaatcaaaaccttatcgatgctaaaaaagggcaagagtcagatttaattctatggctaaagcaatctaaggataatggtatagaactatttaaagccaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaaatctattaaagaaacactatctttattatttgatgatttaaaagctcaaaaacttgatttgagtaaaatttattttaaaaatgataaatctcttactgatctatcacaacaagtttttgatgattatagtgttattggtacagcggtactagaatatataactcaacaaatagcacctaaaaatcttgataaccctagtaagaaagagcaagaattaatagccaaaaaaactgaaaaagcaaaatacttatctctagaaactataaagcttgccttagaagaatttaataagcatagagatatagataaacagtgtaggtttgaagaaatacttgcaaactttgcggctattccgatgatatttgatgaaatagctcaaaacaaagacaatttggcacagatatctatcaaatatcaaaatcaaggtaaaaaagacctacttcaagctagtgcggaagatgatgttaaagctatcaaggatcttttagatcaaactaataatctcttacataaactaaaaatatttcatattagtcagtcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggctaatggttgggataaaaataaagagcctgacaatacggcaattttatttatcaaagatgataaatattatctgggtgtgatgaataagaaaaataacaaaatatttgatgataaagctatcaaagaaaataaaggcgagggttataaaaaaattgtttataaacttttacctggcgcaaataaaatgttacctaaggttttcttttctgctaaatctataaaattttataatcctagtgaagatatacttagaataagaaatcattccacacatacaaaaaatggtagtcctcaaaaaggatatgaaaaatttgagtttaatattgaagattgccgaaaatttatagatttttataaacagtctataagtaagcatccggagtggaaagattttggatttagattttctgatactcaaagatataattctatagatgaattttatagagaagttgaaaatcaaggctacaaactaacttttgaaaatatatcagagagctatattgatagcgtagttaatcagggtaaattgtacctattccaaatctataataaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagctaaagaggcaatagctaataaaaacaaagataatcctaaaaaagagagtgtttttgaatatgatttaatcaaagataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggcaatatcatcaaacaagatactttcaacatcattggtaatgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactaggtaggatcaaaaataatcaagagggcaaaaaactcaatttggttatcaaaaatgaagagtattttgagttcgtgcagaataggaataactaa

SEQ ID NO: 217 - DNA, 3921 bp, Acidaminococcus sp.
  source 1..3921: mol_type = genomic DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 218 - DNA, 3699 bp, Lachnospiracea bacterium
  source 1..3699: mol_type = genomic DNA
atggattacggcaacggccagtttgagcggagagcccccctgaccaagacaatcaccctgcgcctgaagcctatcggcgagacacgggagacaatccgcgagcagaagctgctggagcaggacgccgccttcagaaagctggtggagacagtgacccctatcgtggacgattgtatcaggaagatcgccgataacgccctgtgccactttggcaccgagtatgacttcagctgtctgggcaacgccatctctaagaatgacagcaaggccatcaagaaggagacagagaaggtggagaagctgctggccaaggtgctgaccgagaatctgccagatggcctgcgcaaggtgaacgacatcaattccgccgcctttatccaggatacactgacctctttcgtgcaggacgatgccgacaagcgggtgctgatccaggagctgaagggcaagaccgtgctgatgcagcggttcctgaccacacggatcacagccctgaccgtgtggctgcccgacagagtgttcgagaactttaatatcttcatcgagaacgccgagaagatgagaatcctgctggactcccctctgaatgagaagatcatgaagtttgacccagatgccgagcagtacgcctctctggagttctatggccagtgcctgtctcagaaggacatcgatagctacaacctgatcatctccggcatctatgccgacgatgaggtgaagaaccctggcatcaatgagatcgtgaaggagtacaatcagcagatccggggcgacaaggatgagtccccactgcccaagctgaagaagctgcacaagcagatcctgatgccagtggagaaggccttctttgtgcgcgtgctgtctaacgacagcgatgcccggagcatcctggagaagatcctgaaggacacagagatgctgccctccaagatcatcgaggccatgaaggaggcagatgcaggcgacatcgccgtgtacggcagccggctgcacgagctgagccacgtgatctacggcgatcacggcaagctgtcccagatcatctatgacaaggagtccaagaggatctctgagctgatggagacactgtctccaaaggagcgcaaggagagcaagaagcggctggagggcctggaggagcacatcagaaagtctacatacaccttcgacgagctgaacaggtatgccgagaagaatgtgatggcagcatacatcgcagcagtggaggagtcttgtgccgagatcatgagaaaggagaaggatctgaggaccctgctgagcaaggaggacgtgaagatccggggcaacagacacaatacactgatcgtgaagaactactttaatgcctggaccgtgttccggaacctgatcagaatcctgaggcgcaagtccgaggccgagatcgactctgacttctacgatgtgctggacgattccgtggaggtgctgtctctgacatacaagggcgagaatctgtgccgcagctatatcaccaagaagatcggctccgacctgaagcccgagatcgccacatacggcagcgccctgaggcctaacagccgctggtggtccccaggagagaagtttaatgtgaagttccacaccatcgtgcggagagatggccggctgtactatttcatcctgcccaagggcgccaagcctgtggagctggaggacatggatggcgacatcgagtgtctgcagatgagaaagatccctaacccaacaatctttctgcccaagctggtgttcaaggaccctgaggccttctttagggataatccagaggccgacgagttcgtgtttctgagcggcatgaaggcccccgtgacaatcaccagagagacatacgaggcctacaggtataagctgtataccgtgggcaagctgcgcgatggcgaggtgtccgaagaggagtacaagcgggccctgctgcaggtgctgaccgcctacaaggagtttctggagaacagaatgatctatgccgacctgaatttcggctttaaggatctggaggagtataaggacagctccgagtttatcaagcaggtggagacacacaacaccttcatgtgctgggccaaggtgtctagctcccagctggacgatctggtgaagtctggcaacggcctgctgttcgagatctggagcgagcgcctggagtcctactataagtacggcaatgagaaggtgctgcggggctatgagggcgtgctgctgagcatcctgaaggatgagaacctggtgtccatgcggaccctgctgaacagccggcccatgctggtgtaccggccaaaggagtctagcaagcctatggtggtgcaccgggatggcagcagagtggtggacaggtttgataaggacggcaagtacatcccccctgaggtgcacgacgagctgtatcgcttctttaacaatctgctgatcaaggagaagctgggcgagaaggcccggaagatcctggacaacaagaaggtgaaggtgaaggtgctggagagcgagagagtgaagtggtccaagttctacgatgagcagtttgccgtgaccttcagcgtgaagaagaacgccgattgtctggacaccacaaaggacctgaatgccgaagtgatggagcagtatagcgagtccaacagactgatcctgatcaggaataccacagatatcctgtactatctggtgctggacaagaatggcaaggtgctgaagcagagatccctgaacatcatcaatgacggcgccagggatgtggactggaaggagaggttccgccaggtgacaaaggatagaaacgagggctacaatgagtgggattattccaggacctctaacgacctgaaggaggtgtacctgaattatgccctgaaggagatcgccgaggccgtgatcgagtacaacgccatcctgatcatcgagaagatgtctaatgcctttaaggacaagtatagcttcctggacgacgtgaccttcaagggcttcgagacaaagctgctggccaagctgagcgatctgcactttaggggcatcaaggacggcgagccatgttccttcacaaaccccctgcagctgtgccagaacgattctaataagatcctgcaggacggcgtgatctttatggtgccaaattctatgacacggagcctggaccccgacaccggcttcatctttgccatcaacgaccacaatatcaggaccaagaaggccaagctgaactttctgagcaagttcgatcagctgaaggtgtcctctgagggctgcctgatcatgaagtacagcggcgattccctgcctacacacaacaccgacaatcgcgtgtggaactgctgttgcaatcacccaatcacaaactatgaccgggagacaaagaaggtggagttcatcgaggagcccgtggaggagctgtcccgcgtgctggaggagaatggcatcgagacagacaccgagctgaacaagctgaatgagcgggagaacgtgcctggcaaggtggtggatgccatctactctctggtgctgaattatctgcgcggcacagtgagcggagtggcaggacagagggccgtgtactatagccctgtgaccggcaagaagtacgatatctcctttatccaggccatgaacctgaataggaagtgtgactactataggatcggctccaaggagaggggagagtggaccgatttcgtggcccagctgatcaac

SEQ ID NO: 219 - DNA, 3684 bp, Lachnospiracea bacterium
  source 1..3684: mol_type = genomic DNA
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgataggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcac

SEQ ID NO: 220 - DNA, 3900 bp, Francisella tularensis
  source 1..3900: mol_type = genomic DNA
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 221 - DNA, 4431 bp, Peregrinibacteria bacterium
  source 1..4431: mol_type = genomic DNA
atgtccaacttctttaagaatttcaccaacctgtatgagctgtccaagacactgaggtttgagctgaagcccgtgggcgacaccctgacaaacatgaaggaccacctggagtacgatgagaagctgcagaccttcctgaaggatcagaatatcgacgatgcctatcaggccctgaagcctcagttcgacgagatccacgaggagtttatcacagattctctggagagcaagaaggccaaggagatcgacttctccgagtacctggatctgtttcaggagaagaaggagctgaacgactctgagaagaagctgcgcaacaagatcggcgagacattcaacaaggccggcgagaagtggaagaaggagaagtaccctcagtatgagtggaagaagggctccaagatcgccaatggcgccgacatcctgtcttgccaggatatgctgcagtttatcaagtataagaacccagaggatgagaagatcaagaattacatcgacgatacactgaagggcttctttacctatttcggcggctttaatcagaacagggccaactactatgagacaaagaaggaggcctccaccgcagtggcaacaaggatcgtgcacgagaacctgccaaagttctgtgacaatgtgatccagtttaagcacatcatcaagcggaagaaggatggcaccgtggagaaaaccgagagaaagaccgagtacctgaacgcctaccagtatctgaagaacaataacaagatcacacagatcaaggacgccgagacagagaagatgatcgagtctacacccatcgccgagaagatcttcgacgtgtactacttcagcagctgcctgagccagaagcagatcgaggagtacaaccggatcatcggccactataatctgctgatcaacctgtataaccaggccaagagatctgagggcaagcacctgagcgccaacgagaagaagtataaggacctgcctaagttcaagaccctgtataagcagatcggctgcggcaagaagaaggacctgttttacacaatcaagtgtgataccgaggaggaggccaataagtcccggaacgagggcaaggagtcccactctgtggaggagatcatcaacaaggcccaggaggccatcaataagtacttcaagtctaataacgactgtgagaatatcaacaccgtgcccgacttcatcaactatatcctgacaaaggagaattacgagggcgtgtattggagcaaggccgccatgaacaccatctccgacaagtacttcgccaattatcacgacctgcaggatagactgaaggaggccaaggtgtttcagaaggccgataagaagtccgaggacgatatcaagatcccagaggccatcgagctgtctggcctgttcggcgtgctggacagcctggccgattggcagaccacactgtttaagtctagcatcctgagcaacgaggacaagctgaagatcatcacagattcccagaccccctctgaggccctgctgaagatgatcttcaatgacatcgagaagaacatggagtcctttctgaaggagacaaacgatatcatcaccctgaagaagtataagggcaataaggagggcaccgagaagatcaagcagtggttcgactatacactggccatcaaccggatgctgaagtactttctggtgaaggagaataagatcaagggcaactccctggataccaatatctctgaggccctgaaaaccctgatctacagcgacgatgccgagtggttcaagtggtacgacgccctgagaaactatctgacccagaagcctcaggatgaggccaaggagaataagctgaagctgaatttcgacaacccatctctggccggcggctgggatgtgaacaaggagtgcagcaatttttgcgtgatcctgaaggacaagaacgagaagaagtacctggccatcatgaagaagggcgagaataccctgttccagaaggagtggacagagggccggggcaagaacctgacaaagaagtctaatccactgttcgagatcaataactgcgagatcctgagcaagatggagtatgacttttgggccgacgtgagcaagatgatccccaagtgtagcacccagctgaaggccgtggtgaaccacttcaagcagtccgacaatgagttcatctttcctatcggctacaaggtgacaagcggcgagaagtttagggaggagtgcaagatctccaagcaggacttcgagctgaataacaaggtgtttaataagaacgagctgagcgtgaccgccatgcgctacgatctgtcctctacacaggagaagcagtatatcaaggccttccagaaggagtactgggagctgctgtttaagcaggagaagcgggacaccaagctgacaaataacgagatcttcaacgagtggatcaatttttgcaacaagaagtatagcgagctgctgtcctgggagagaaagtacaaggatgccctgaccaattggatcaacttctgtaagtactttctgagcaagtatcccaagaccacactgttcaactactcttttaaggagagcgagaattataactccctggacgagttctaccgggacgtggatatctgttcttacaagctgaatatcaacaccacaatcaataagagcatcctggatagactggtggaggagggcaagctgtacctgtttgagatcaagaatcaggacagcaacgatggcaagtccatcggccacaagaataacctgcacaccatctactggaacgccatcttcgagaattttgacaacaggcctaagctgaatggcgaggccgagatcttctatcgcaaggccatctccaaggataagctgggcatcgtgaagggcaagaaaaccaagaacggcaccgagatcatcaagaattacagattcagcaaggagaagtttatcctgcacgtgccaatcaccctgaacttctgctccaataacgagtatgtgaatgacatcgtgaacacaaagttctacaatttttccaacctgcactttctgggcatcgataggggcgagaagcacctggcctactattctctggtgaataagaacggcgagatcgtggaccagggcacactgaacctgcctttcaccgacaaggatggcaatcagcgcagcatcaagaaggagaagtacttttataacaagcaggaggacaagtgggaggccaaggaggtggattgttggaattataacgacctgctggatgccatggcctctaaccgggacatggccagaaagaattggcagaggatcggcaccatcaaggaggccaagaacggctacgtgagcctggtcatcaggaagatcgccgatctggccgtgaataacgagcgccccgccttcatcgtgctggaggacctgaatacaggctttaagcggtccagacagaagatcgataagagcgtgtaccagaagttcgagctggccctggccaagaagctgaactttctggtggacaagaatgccaagcgcgatgagatcggctcccctacaaaggccctgcagctgaccccccctgtgaataactacggcgacattgagaacaagaagcaggccggcatcatgctgtatacccgggccaattatacctctcagacagatccagccacaggctggagaaagaccatctatctgaaggccggccccgaggagacaacatacaagaaggacggcaagatcaagaacaagagcgtgaaggaccagatcatcgagacattcaccgatatcggctttgacggcaaggattactatttcgagtacgacaagggcgagtttgtggatgagaaaaccggcgagatcaagcccaagaagtggcggctgtactccggcgagaatggcaagtccctggacaggttccgcggagagagggagaaggataagtatgagtggaagatcgacaagatcgatatcgtgaagatcctggacgatctgttcgtgaattttgacaagaacatcagcctgctgaagcagctgaaggagggcgtggagctgacccggaataacgagcacggcacaggcgagtccctgagattcgccatcaacctgatccagcagatccggaataccggcaataacgagagagacaacgatttcatcctgtccccagtgagggacgagaatggcaagcactttgactctcgcgagtactgggataaggagacaaagggcgagaagatcagcatgcccagctccggcgatgccaatggcgccttcaacatcgcccggaagggcatcatcatgaacgcccacatcctggccaatagcgactccaaggatctgtccctgttcgtgtctgacgaggagtgggatctgcacctgaataacaagaccgagtggaagaagcagctgaacatcttttctagcaggaaggccatggccaagcgcaagaag

SEQ ID NO: 222 - DNA, 4056 bp, Parcubacteria bacterium
  source 1..4056: mol_type = genomic DNA
atggagaacatcttcgaccagtttatcggcaagtacagcctgtccaagaccctgagattcgagctgaagcccgtgggcaagacagaggacttcctgaagatcaacaaggtgtttgagaaggatcagaccatcgacgatagctacaatcaggccaagttctattttgattccctgcaccagaagtttatcgacgccgccctggcctccgataagacatccgagctgtctttccagaactttgccgacgtgctggagaagcagaataagatcatcctggataagaagagagagatgggcgccctgaggaagcgcgacaagaacgccgtgggcatcgataggctgcagaaggagatcaatgacgccgaggatatcatccagaaggagaaggagaagatctacaaggacgtgcgcaccctgttcgataacgaggccgagtcttggaaaacctactatcaggagcgggaggtggacggcaagaagatcaccttcagcaaggccgacctgaagcagaagggcgccgattttctgacagccgccggcatcctgaaggtgctgaagtatgagttccccgaggagaaggagaaggagtttcaggccaagaaccagccctccctgttcgtggaggagaaggagaatcctggccagaagaggtacatcttcgactcttttgataagttcgccggctatctgaccaagtttcagcagacaaagaagaatctgtacgcagcagacggcaccagcacagcagtggccacccgcatcgccgataactttatcatcttccaccagaataccaaggtgttccgggacaagtacaagaacaatcacacagacctgggcttcgatgaggagaacatctttgagatcgagaggtataagaattgcctgctgcagcgcgagatcgagcacatcaagaatgagaatagctacaacaagatcatcggccggatcaataagaagatcaaggagtatcgggaccagaaggccaaggataccaagctgacaaagtccgacttccctttctttaagaacctggataagcagatcctgggcgaggtggagaaggagaagcagctgatcgagaaaacccgggagaaaaccgaggaggacgtgctgatcgagcggttcaaggagttcatcgagaacaatgaggagaggttcaccgccgccaagaagctgatgaatgccttctgtaacggcgagtttgagtccgagtacgagggcatctatctgaagaataaggccatcaacacaatctcccggagatggttcgtgtctgacagagattttgagctgaagctgcctcagcagaagtccaagaacaagtctgagaagaatgagccaaaggtgaagaagttcatctccatcgccgagatcaagaacgccgtggaggagctggacggcgatatctttaaggccgtgttctacgacaagaagatcatcgcccagggcggctctaagctggagcagttcctggtcatctggaagtacgagtttgagtatctgttccgggacatcgagagagagaacggcgagaagctgctgggctatgatagctgcctgaagatcgccaagcagctgggcatcttcccacaggagaaggaggcccgcgagaaggcaaccgccgtgatcaagaattacgccgacgccggcctgggcatcttccagatgatgaagtatttttctctggacgataaggatcggaagaacacccccggccagctgagcacaaatttctacgccgagtatgacggctactacaaggatttcgagtttatcaagtactacaacgagtttaggaacttcatcaccaagaagcctttcgacgaggataagatcaagctgaactttgagaatggcgccctgctgaagggctgggacgagaacaaggagtacgatttcatgggcgtgatcctgaagaaggagggccgcctgtatctgggcatcatgcacaagaaccaccggaagctgtttcagtccatgggcaatgccaagggcgacaacgccaatagataccagaagatgatctataagcagatcgccgacgcctctaaggatgtgcccaggctgctgctgaccagcaagaaggccatggagaagttcaagccttcccaggagatcctgagaatcaagaaggagaaaaccttcaagcgggagagcaagaacttttccctgagagatctgcacgccctgatcgagtactataggaactgcatccctcagtacagcaattggtccttttatgacttccagtttcaggataccggcaagtaccagaatatcaaggagttcacagacgatgtgcagaagtacggctataagatctcctttcgcgacatcgacgatgagtatatcaatcaggccctgaacgagggcaagatgtacctgttcgaggtggtgaacaaggatatctataacaccaagaatggctccaagaatctgcacacactgtactttgagcacatcctgtctgccgagaacctgaatgacccagtgttcaagctgtctggcatggccgagatctttcagcggcagcccagcgtgaacgaaagagagaagatcaccacacagaagaatcagtgtatcctggacaagggcgatagagcctacaagtataggcgctacaccgagaagaagatcatgttccacatgagcctggtgctgaacacaggcaagggcgagatcaagcaggtgcagtttaataagatcatcaaccagaggatcagctcctctgacaacgagatgagggtgaatgtgatcggcatcgatcgcggcgagaagaacctgctgtactatagcgtggtgaagcagaatggcgagatcatcgagcaggcctccctgaacgagatcaatggcgtgaactaccgggacaagctgatcgagagggagaaggagcgcctgaagaaccggcagagctggaagcctgtggtgaagatcaaggatctgaagaagggctacatctcccacgtgatccacaagatctgccagctgatcgagaagtattctgccatcgtggtgctggaggacctgaatatgagattcaagcagatcaggggaggaatcgagcggagcgtgtaccagcagttcgagaaggccctgatcgataagctgggctatctggtgtttaaggacaacagggatctgagggcaccaggaggcgtgctgaatggctaccagctgtctgccccctttgtgagcttcgagaagatgcgcaagcagaccggcatcctgttctacacacaggccgagtataccagcaagacagacccaatcaccggctttcggaagaacgtgtatatctctaatagcgcctccctggataagatcaaggaggccgtgaagaagttcgacgccatcggctgggatggcaaggagcagtcttacttctttaagtacaacccttacaacctggccgacgagaagtataagaactctaccgtgagcaaggagtgggccatctttgccagcgccccaagaatccggagacagaagggcgaggacggctactggaagtatgatagggtgaaagtgaatgaggagttcgagaagctgctgaaggtctggaattttgtgaacccaaaggccacagatatcaagcaggagatcatcaagaaggagaaggcaggcgacctgcagggagagaaggagctggatggccggctgagaaacttttggcactctttcatctacctgtttaacctggtgctggagctgcgcaattctttcagcctgcagatcaagatcaaggcaggagaagtgatcgcagtggacgagggcgtggacttcatcgccagcccagtgaagcccttctttaccacacccaacccttacatcccctccaacctgtgctggctggccgtggagaatgcagacgcaaacggagcctataatatcgccaggaagggcgtgatgatcctgaagaagatccgcgagcacgccaagaaggaccccgagttcaagaagctgccaaacctgtttatcagcaatgcagagtgggacgaggcagcccgggattggggcaagtacgcaggcaccacagccctgaacctggaccac

SEQ ID NO: 223 - DNA, 3618 bp, Lachnospiraceae bacterium
  source 1..3618: mol_type = genomic DNA
atgtactatgagtccctgaccaagcagtaccccgtgtctaagacaatccggaatgagctgatccctatcggcaagacactggataacatccgccagaacaatatcctggagagcgacgtgaagcggaagcagaactacgagcacgtgaagggcatcctggatgagtatcacaagcagctgatcaacgaggccctggacaattgcaccctgccatccctgaagatcgccgccgagatctacctgaagaatcagaaggaggtgtctgacagagaggatttcaacaagacacaggacctgctgaggaaggaggtggtggagaagctgaaggcccacgagaactttaccaagatcggcaagaaggacatcctggatctgctggagaagctgccttccatctctgaggacgattacaatgccctggagagcttccgcaacttttacacctatttcacatcctacaacaaggtgcgggagaatctgtattctgataaggagaagagctccacagtggcctacagactgatcaacgagaatttcccaaagtttctggacaatgtgaagagctataggtttgtgaaaaccgcaggcatcctggcagatggcctgggagaggaggagcaggactccctgttcatcgtggagacattcaacaagaccctgacacaggacggcatcgatacctacaattctcaagtgggcaagatcaactctagcatcaatctgtataaccagaagaatcagaaggccaatggcttcagaaagatccccaagatgaagatgctgtataagcagatcctgtccgatagggaggagtctttcatcgacgagtttcagagcgatgaggtgctgatcgacaacgtggagtcttatggcagcgtgctgatcgagtctctgaagtcctctaaggtgagcgccttctttgatgccctgagagagtctaagggcaagaacgtgtacgtgaagaatgacctggccaagacagccatgagcaacatcgtgttcgagaattggaggacctttgacgatctgctgaaccaggagtacgacctggccaacgagaacaagaagaaggacgataagtatttcgagaagcgccagaaggagctgaagaagaataagagctactccctggagcacctgtgcaacctgtccgaggattcttgtaacctgatcgagaattatatccaccagatctccgacgatatcgagaatatcatcatcaacaatgagacattcctgcgcatcgtgatcaatgagcacgacaggtcccgcaagctggccaagaaccggaaggccgtgaaggccatcaaggactttctggattctatcaaggtgctggagcgggagctgaagctgatcaacagctccggccaggagctggagaaggatctgatcgtgtactctgcccacgaggagctgctggtggagctgaagcaggtggacagcctgtataacatgaccagaaattatctgacaaagaagcctttctctaccgagaaggtgaagctgaactttaatcgcagcacactgctgaacggctgggatcggaataaggagacagacaacctgggcgtgctgctgctgaaggacggcaagtactatctgggcatcatgaacacaagcgccaataaggccttcgtgaatccccctgtggccaagaccgagaaggtgtttaagaaggtggattacaagctgctgccagtgcccaaccagatgctgccaaaggtgttctttgccaagagcaatatcgacttctataacccctctagcgagatctactccaattataagaagggcacccacaagaagggcaatatgttttccctggaggattgtcacaacctgatcgacttctttaaggagtctatcagcaagcacgaggactggagcaagttcggctttaagttcagcgatacagcctcctacaacgacatctccgagttctatcgcgaggtggagaagcagggctacaagctgacctatacagacatcgatgagacatacatcaatgatctgatcgagcggaacgagctgtacctgttccagatctataataaggactttagcatgtactccaagggcaagctgaacctgcacacactgtatttcatgatgctgtttgatcagcgcaatatcgacgacgtggtgtataagctgaacggagaggcagaggtgttctataggccagcctccatctctgaggacgagctgatcatccacaaggccggcgaggagatcaagaacaagaatcctaaccgggccagaaccaaggagacaagcaccttcagctacgacatcgtgaaggataagcggtatagcaaggataagtttaccctgcacatccccatcacaatgaacttcggcgtggatgaggtgaagcggttcaacgacgccgtgaacagcgccatccggatcgatgagaatgtgaacgtgatcggcatcgaccggggcgagagaaatctgctgtacgtggtggtcatcgactctaagggcaacatcctggagcagatctccctgaactctatcatcaataaggagtacgacatcgagacagattatcacgcactgctggatgagagggagggcggcagagataaggcccggaaggactggaacaccgtggagaatatcagggacctgaaggccggctacctgagccaggtggtgaacgtggtggccaagctggtgctgaagtataatgccatcatctgcctggaggacctgaactttggcttcaagaggggccgccagaaggtggagaagcaggtgtaccagaagttcgagaagatgctgatcgataagctgaattacctggtcatcgacaagagccgcgagcagacatcccctaaggagctgggaggcgccctgaacgcactgcagctgacctctaagttcaagagctttaaggagctgggcaagcagtccggcgtgatctactatgtgcctgcctacctgacctctaagatcgatccaaccacaggcttcgccaatctgttttatatgaagtgtgagaacgtggagaagtccaagagattctttgacggctttgatttcatcaggttcaacgccctggagaacgtgttcgagttcggctttgactaccggagcttcacccagagggcctgcggcatcaattccaagtggaccgtgtgcaccaacggcgagcgcatcatcaagtatcggaatccagataagaacaatatgttcgacgagaaggtggtggtggtgaccgatgagatgaagaacctgtttgagcagtacaagatcccctatgaggatggcagaaatgtgaaggacatgatcatcagcaacgaggaggccgagttctaccggagactgtataggctgctgcagcagaccctgcagatgagaaacagcacctccgacggcacaagggattacatcatctcccctgtgaagaataagagagaggcctacttcaacagcgagctgtccgacggctctgtgccaaaggacgccgatgccaacggcgcctacaatatcgccagaaagggcctgtgggtgctggagcagatcaggcagaagagcgagggcgagaagatcaatctggccatgaccaacgccgagtggctggagtatgcccagacacacctgctg

SEQ ID NO: 224 - DNA, 3714 bp, Candidatus Methanoplasma termitum
  source 1..3714: mol_type = genomic DNA
atgaacaattacgacgagttcaccaagctgtatcctatccagaaaaccatccggtttgagctgaagccacagggcagaaccatggagcacctggagacattcaacttctttgaggaggaccgggatagagccgagaagtataagatcctgaaggaggccatcgacgagtaccacaagaagtttatcgatgagcacctgaccaatatgtccctggattggaactctctgaagcagatcagcgagaagtactataagagcagggaggagaaggacaagaaggtgttcctgtccgagcagaagaggatgcgccaggagatcgtgtctgagtttaagaaggacgatcgcttcaaggacctgttttccaagaagctgttctctgagctgctgaaggaggagatctacaagaagggcaaccaccaggagatcgacgccctgaagagcttcgataagttttccggctatttcatcggcctgcacgagaataggaagaacatgtactccgacggcgatgagatcaccgccatctccaatcgcatcgtgaatgagaacttccccaagtttctggataacctgcagaagtaccaggaggccaggaagaagtatcctgagtggatcatcaaggccgagagcgccctggtggcccacaatatcaagatggacgaggtgttctccctggagtactttaataaggtgctgaaccaggagggcatccagcggtacaacctggccctgggcggctatgtgaccaagagcggcgagaagatgatgggcctgaatgatgccctgaacctggcccaccagtccgagaagagctccaagggcagaatccacatgacccccctgttcaagcagatcctgtccgagaaggagtccttctcttacatccccgacgtgtttacagaggattctcagctgctgcctagcatcggcggcttctttgcccagatcgagaatgacaaggatggcaacatcttcgaccgggccctggagctgatctctagctacgccgagtatgataccgagcggatctatatcagacaggccgacatcaatagagtgtccaacgtgatctttggagagtggggcaccctgggaggcctgatgagggagtacaaggccgactctatcaatgatatcaacctggagcgcacatgcaagaaggtggacaagtggctggattctaaggagtttgccctgagcgatgtgctggaggccatcaagaggaccggcaacaatgacgccttcaacgagtatatctccaagatgcggacagccagagagaagatcgatgccgcccgcaaggagatgaagttcatcagcgagaagatctccggcgatgaggagtctatccacatcatcaagaccctgctggacagcgtgcagcagttcctgcacttctttaatctgtttaaggcaaggcaggacatcccactggatggagccttctacgccgagtttgacgaggtgcacagcaagctgtttgccatcgtgcccctgtataacaaggtgcggaactatctgaccaagaacaatctgaacacaaagaagatcaagctgaatttcaagaaccctacactggccaatggctgggaccagaacaaggtgtacgattatgcctccctgatctttctgcgggacggcaattactatctgggcatcatcaatcctaagagaaagaagaacatcaagttcgagcagggctctggcaacggccccttctaccggaagatggtgtataagcagatccccggccctaataagaacctgccaagagtgttcctgacctccacaaagggcaagaaggagtataagccctctaaggagatcatcgagggctacgaggccgacaagcacatcaggggcgataagttcgacctggatttttgtcacaagctgatcgatttctttaaggagtccatcgagaagcacaaggactggtctaagttcaacttctacttcagcccaaccgagagctatggcgacatctctgagttctacctggatgtggagaagcagggctatcgcatgcactttgagaatatcagcgccgagacaatcgacgagtatgtggagaagggcgatctgtttctgttccagatctacaacaaggattttgtgaaggccgccaccggcaagaaggacatgcacacaatctactggaatgccgccttcagccccgagaacctgcaggacgtggtggtgaagctgaacggcgaggccgagctgttttatagggacaagtccgatatcaaggagatcgtgcaccgcgagggcgagatcctggtgaataggacctacaacggccgcacaccagtgcccgacaagatccacaagaagctgaccgattatcacaatggccggacaaaggacctgggcgaggccaaggagtacctggataaggtgagatacttcaaggcccactatgacatcaccaaggatcggagatacctgaacgacaagatctatttccacgtgcctctgaccctgaacttcaaggccaacggcaagaagaatctgaacaagatggtcatcgagaagttcctgtccgatgagaaggcccacatcatcggcatcgacaggggcgagcgcaatctgctgtactattccatcatcgacaggtctggcaagatcatcgatcagcagagcctgaatgtgatcgacggctttgattatcgggagaagctgaaccagagagagatcgagatgaaggatgcccgccagtcttggaacgccatcggcaagatcaaggacctgaaggagggctacctgagcaaggccgtgcacgagatcaccaagatggccatccagtataatgccatcgtggtcatggaggagctgaactacggcttcaagcggggccggttcaaggtggagaagcagatctatcagaagttcgagaatatgctgatcgataagatgaactacctggtgtttaaggacgcacctgatgagtccccaggaggcgtgctgaatgcctaccagctgacaaacccactggagtctttcgccaagctgggcaagcagaccggcatcctgttttacgtgccagccgcctatacatccaagatcgaccccaccacaggcttcgtgaatctgtttaacacctcctctaagacaaacgcccaggagcggaaggagttcctgcagaagtttgagagcatctcctattctgccaaggatggcggcatctttgccttcgcctttgactacagaaagttcggcaccagcaagacagatcacaagaacgtgtggaccgcctatacaaacggcgagaggatgcgctacatcaaggagaagaagcggaatgagctgtttgacccttctaaggagatcaaggaggccctgaccagctccggcatcaagtacgatggcggccagaacatcctgccagacatcctgaggagcaacaataacggcctgatctacacaatgtattctagcttcatcgccgccatccagatgcgcgtgtacgacggcaaggaggattatatcatcagccccatcaagaactccaagggcgagttctttaggaccgaccccaagaggcgcgagctgcctatcgacgccgatgccaatggcgcctacaacatcgccctgaggggagagctgacaatgagggcaatcgcagagaagttcgaccctgatagcgagaagatggccaagctggagctgaagcacaaggattggttcgagtttatgcagaccagaggcgac

SEQ ID NO: 225 - DNA, 3846 bp, Eubacterium eligens
  source 1..3846: mol_type = genomic DNA
atgaacggcaataggtccatcgtgtaccgcgagttcgtgggcgtgatccccgtggccaagaccctgaggaatgagctgcgccctgtgggccacacacaggagcacatcatccagaacggcctgatccaggaggacgagctgcggcaggagaagagcaccgagctgaagaacatcatggacgattactatagagagtacatcgataagtctctgagcggcgtgaccgacctggacttcaccctgctgttcgagctgatgaacctggtgcagagctccccctccaaggacaataagaaggccctggagaaggagcagtctaagatgagggagcagatctgcacccacctgcagtccgactctaactacaagaatatctttaacgccaagctgctgaaggagatcctgcctgatttcatcaagaactacaatcagtatgacgtgaaggataaggccggcaagctggagacactggccctgtttaatggcttcagcacatactttaccgacttctttgagaagaggaagaacgtgttcaccaaggaggccgtgagcacatccatcgcctaccgcatcgtgcacgagaactccctgatcttcctggccaatatgacctcttataagaagatcagcgagaaggccctggatgagatcgaagtgatcgagaagaacaatcaggacaagatgggcgattgggagctgaatcagatctttaaccctgacttctacaatatggtgctgatccagtccggcatcgacttctacaacgagatctgcggcgtggtgaatgcccacatgaacctgtactgtcagcagaccaagaacaattataacctgttcaagatgcggaagctgcacaagcagatcctggcctacaccagcaccagcttcgaggtgcccaagatgttcgaggacgatatgagcgtgtataacgccgtgaacgccttcatcgacgagacagagaagggcaacatcatcggcaagctgaaggatatcgtgaataagtacgacgagctggatgagaagagaatctatatcagcaaggacttttacgagacactgagctgcttcatgtccggcaactggaatctgatcacaggctgcgtggagaacttctacgatgagaacatccacgccaagggcaagtccaaggaggagaaggtgaagaaggccgtgaaggaggacaagtacaagtctatcaatgacgtgaacgatctggtggagaagtatatcgatgagaaggagaggaatgagttcaagaacagcaatgccaagcagtacatccgcgagatctccaacatcatcaccgacacagagacagcccacctggagtatgacgatcacatctctctgatcgagagcgaggagaaggccgacgagatgaagaagcggctggatatgtatatgaacatgtaccactgggccaaggcctttatcgtggacgaggtgctggacagagatgagatgttctacagcgatatcgacgatatctataatatcctggagaacatcgtgccactgtataatcgggtgagaaactacgtgacccagaagccctacaactctaagaagatcaagctgaatttccagagccctacactggccaatggctggtcccagtctaaggagttcgacaacaatgccatcatcctgatcagagataacaagtactatctggccatcttcaatgccaagaacaagccagacaagaagatcatccagggcaactccgataagaagaacgacaacgattacaagaagatggtgtataacctgctgccaggcgccaacaagatgctgcccaaggtgtttctgtctaagaagggcatcgagacattcaagccctccgactatatcatctctggctacaacgcccacaagcacatcaagacaagcgagaattttgatatctccttctgtcgggacctgatcgattacttcaagaacagcatcgagaagcacgccgagtggagaaagtatgagttcaagttttccgccaccgacagctactccgatatctctgagttctatcgggaggtggagatgcagggctacagaatcgactggacatatatcagcgaggccgacatcaacaagctggatgaggagggcaagatctatctgtttcagatctacaataaggatttcgccgagaacagcaccggcaaggagaatctgcacacaatgtactttaagaacatcttctccgaggagaatctgaaggacatcatcatcaagctgaacggccaggccgagctgttttatcggagagcctctgtgaagaatcccgtgaagcacaagaaggatagcgtgctggtgaacaagacctacaagaatcagctggacaacggcgacgtggtgagaatccccatccctgacgatatctataacgagatctacaagatgtataatggctacatcaaggagtccgacctgtctgaggccgccaaggagtacctggataaggtggaggtgaggaccgcccagaaggacatcgtgaaggattaccgctatacagtggacaagtacttcatccacacacctatcaccatcaactataaggtgaccgcccgcaacaatgtgaatgatatggtggtgaagtacatcgcccagaacgacgatatccacgtgatcggcatcgaccggggcgagagaaacctgatctacatctccgtgatcgattctcacggcaacatcgtgaagcagaaatcctacaacatcctgaacaactacgactacaagaagaagctggtggagaaggagaaaacccgggagtacgccagaaagaactggaagagcatcggcaatatcaaggagctgaaggagggctatatctccggcgtggtgcacgagatcgccatgctgatcgtggagtacaacgccatcatcgccatggaggacctgaattatggctttaagaggggccgcttcaaggtggagcggcaggtgtaccagaagtttgagagcatgctgatcaataagctgaactatttcgccagcaaggagaagtccgtggacgagccaggaggcctgctgaagggctatcagctgacctacgtgcccgataatatcaagaacctgggcaagcagtgcggcgtgatcttttacgtgcctgccgccttcaccagcaagatcgacccatccacaggctttatctctgccttcaactttaagtctatcagcacaaatgcctctcggaagcagttctttatgcagtttgacgagatcagatactgtgccgagaaggatatgttcagctttggcttcgactacaacaacttcgatacctacaacatcacaatgggcaagacacagtggaccgtgtatacaaacggcgagagactgcagtctgagttcaacaatgccaggcgcaccggcaagacaaagagcatcaatctgacagagacaatcaagctgctgctggaggacaatgagatcaactacgccgacggccacgatatcaggatcgatatggagaagatggacgaggataagaagagcgagttctttgcccagctgctgagcctgtataagctgaccgtgcagatgcgcaattcctatacagaggccgaggagcaggagaacggcatctcttacgacaagatcatcagccctgtgatcaatgatgagggcgagttctttgactccgataactataaggagtctgacgataaggagtgcaagatgccaaaggacgccgatgccaacggcgcctactgtatcgccctgaagggcctgtatgaggtgctgaagatcaagagcgagtggaccgaggacggctttgataggaattgcctgaagctgccacacgcagagtggctggacttcatccagaacaagcggtacgag

SEQ ID NO: 226 - DNA, 4119 bp, Moraxella bovoculi
  source 1..4119: mol_type = genomic DNA
atgctgttccaggactttacccacctgtatccactgtccaagacagtgagatttgagctgaagcccatcgataggaccctggagcacatccacgccaagaacttcctgtctcaggacgagacaatggccgatatgcaccagaaggtgaaagtgatcctggacgattaccaccgcgacttcatcgccgatatgatgggcgaggtgaagctgaccaagctggccgagttctatgacgtgtacctgaagtttcggaagaacccaaaggacgatgagctgcagaagcagctgaaggatctgcaggccgtgctgagaaaggagatcgtgaagcccatcggcaatggcggcaagtataaggccggctacgacaggctgttcggcgccaagctgtttaaggacggcaaggagctgggcgatctggccaagttcgtgatcgcacaggagggagagagctccccaaagctggcccacctggcccacttcgagaagttttccacctatttcacaggctttcacgataaccggaagaatatgtattctgacgaggataagcacaccgccatcgcctaccgcctgatccacgagaacctgccccggtttatcgacaatctgcagatcctgaccacaatcaagcagaagcactctgccctgtacgatcagatcatcaacgagctgaccgccagcggcctggacgtgtctctggccagccacctggatggctatcacaagctgctgacacaggagggcatcaccgcctacaatacactgctgggaggaatctccggagaggcaggctctcctaagatccagggcatcaacgagctgatcaattctcaccacaaccagcactgccacaagagcgagagaatcgccaagctgaggccactgcacaagcagatcctgtccgacggcatgagcgtgtccttcctgccctctaagtttgccgacgatagcgagatgtgccaggccgtgaacgagttctatcgccactacgccgacgtgttcgccaaggtgcagagcctgttcgacggctttgacgatcaccagaaggatggcatctacgtggagcacaagaacctgaatgagctgtccaagcaggccttcggcgactttgcactgctgggacgcgtgctggacggatactatgtggatgtggtgaatccagagttcaacgagcggtttgccaaggccaagaccgacaatgccaaggccaagctgacaaaggagaaggataagttcatcaagggcgtgcactccctggcctctctggagcaggccatcgagcactataccgcaaggcacgacgatgagagcgtgcaggcaggcaagctgggacagtacttcaagcacggcctggccggagtggacaaccccatccagaagatccacaacaatcacagcaccatcaagggctttctggagagggagcgccctgcaggagagagagccctgccaaagatcaagtccggcaagaatcctgagatgacacagctgaggcagctgaaggagctgctggataacgccctgaatgtggcccacttcgccaagctgctgaccacaaagaccacactggacaatcaggatggcaacttctatggcgagtttggcgtgctgtacgacgagctggccaagatccccaccctgtataacaaggtgagagattacctgagccagaagcctttctccaccgagaagtacaagctgaactttggcaatccaacactgctgaatggctgggacctgaacaaggagaaggataatttcggcgtgatcctgcagaaggacggctgctactatctggccctgctggacaaggcccacaagaaggtgtttgataacgcccctaatacaggcaagagcatctatcagaagatgatctataagtacctggaggtgaggaagcagttccccaaggtgttcttttccaaggaggccatcgccatcaactaccacccttctaaggagctggtggagatcaaggacaagggccggcagagatccgacgatgagcgcctgaagctgtatcggtttatcctggagtgtctgaagatccaccctaagtacgataagaagttcgagggcgccatcggcgacatccagctgtttaagaaggataagaagggcagagaggtgccaatcagcgagaaggacctgttcgataagatcaacggcatcttttctagcaagcctaagctggagatggaggacttctttatcggcgagttcaagaggtataacccaagccaggacctggtggatcagtataatatctacaagaagatcgactccaacgataatcgcaagaaggagaatttctacaacaatcaccccaagtttaagaaggatctggtgcggtactattacgagtctatgtgcaagcacgaggagtgggaggagagcttcgagttttccaagaagctgcaggacatcggctgttacgtggatgtgaacgagctgtttaccgagatcgagacacggagactgaattataagatctccttctgcaacatcaatgccgactacatcgatgagctggtggagcagggccagctgtatctgttccagatctacaacaaggacttttccccaaaggcccacggcaagcccaatctgcacaccctgtacttcaaggccctgttttctgaggacaacctggccgatcctatctataagctgaatggcgaggcccagatcttctacagaaaggcctccctggacatgaacgagacaacaatccacagggccggcgaggtgctggagaacaagaatcccgataatcctaagaagagacagttcgtgtacgacatcatcaaggataagaggtacacacaggacaagttcatgctgcacgtgccaatcaccatgaactttggcgtgcagggcatgacaatcaaggagttcaataagaaggtgaaccagtctatccagcagtatgacgaggtgaacgtgatcggcatcgatcggggcgagagacacctgctgtacctgaccgtgatcaatagcaagggcgagatcctggagcagtgttccctgaacgacatcaccacagcctctgccaatggcacacagatgaccacaccttaccacaagatcctggataagagggagatcgagcgcctgaacgcccgggtgggatggggcgagatcgagacaatcaaggagctgaagtctggctatctgagccacgtggtgcaccagatcagccagctgatgctgaagtacaacgccatcgtggtgctggaggacctgaatttcggctttaagaggggccgctttaaggtggagaagcagatctatcagaacttcgagaatgccctgatcaagaagctgaaccacctggtgctgaaggacaaggccgacgatgagatcggctcttacaagaatgccctgcagctgaccaacaatttcacagatctgaagagcatcggcaagcagaccggcttcctgttttatgtgcccgcctggaacacctctaagatcgaccctgagacaggctttgtggatctgctgaagccaagatacgagaacatcgcccagagccaggccttctttggcaagttcgacaagatctgctataatgccgacaaggattacttcgagtttcacatcgactacgccaagtttaccgataaggccaagaatagccgccagatctggacaatctgttcccacggcgacaagcggtacgtgtacgataagacagccaaccagaataagggcgccgccaagggcatcaacgtgaatgatgagctgaagtccctgttcgcccgccaccacatcaacgagaagcagcccaacctggtcatggacatctgccagaacaatgataaggagtttcacaagtctctgatgtacctgctgaaaaccctgctggccctgcggtacagcaacgcctcctctgacgaggatttcatcctgtcccccgtggcaaacgacgagggcgtgttctttaatagcgccctggccgacgatacacagcctcagaatgccgatgccaacggcgcctaccacatcgccctgaagggcctgtggctgctgaatgagctgaagaactccgacgatctgaacaaggtgaagctggccatcgacaatcagacctggctgaatttcgcccagaacagg

SEQ ID NO: 227 - DNA, 3969 bp, Prevotella disiens
  source 1..3969: mol_type = genomic DNA
atggagaactatcaggagttcaccaacctgtttcagctgaataagacactgagattcgagctgaagcccatcggcaagacctgcgagctgctggaggagggcaagatcttcgccagcggctcctttctggagaaggacaaggtgagggccgataacgtgagctacgtgaagaaggagatcgacaagaagcacaagatctttatcgaggagacactgagctccttctctatcagcaacgatctgctgaagcagtactttgactgctataatgagctgaaggccttcaagaaggactgtaagagcgatgaggaggaggtgaagaaaaccgccctgcgcaacaagtgtacctccatccagagggccatgcgcgaggccatctctcaggcctttctgaagagcccccagaagaagctgctggccatcaagaacctgatcgagaacgtgttcaaggccgacgagaatgtgcagcacttctccgagtttaccagctatttctccggctttgagacaaacagagagaatttctactctgacgaggagaagtccacatctatcgcctataggctggtgcacgataacctgcctatcttcatcaagaacatctacatcttcgagaagctgaaggagcagttcgacgccaagaccctgagcgagatcttcgagaactacaagctgtatgtggccggctctagcctggatgaggtgttctccctggagtactttaacaataccctgacacagaagggcatcgacaactataatgccgtgatcggcaagatcgtgaaggaggataagcaggagatccagggcctgaacgagcacatcaacctgtataatcagaagcacaaggaccggagactgcccttctttatctccctgaagaagcagatcctgtccgatcgggaggccctgtcttggctgcctgacatgttcaagaatgattctgaagtgatcaaggccctgaagggcttctacatcgaggacggctttgagaacaatgtgctgacacctctggccaccctgctgtcctctctggataagtacaacctgaatggcatctttatccgcaacaatgaggccctgagctccctgtcccagaacgtgtatcggaatttttctatcgacgaggccatcgatgccaacgccgagctgcagaccttcaacaattacgagctgatcgccaatgccctgcgcgccaagatcaagaaggagacaaagcagggccggaagtctttcgagaagtacgaggagtatatcgataagaaggtgaaggccatcgacagcctgtccatccaggagatcaacgagctggtggagaattacgtgagcgagtttaactctaatagcggcaacatgccaagaaaggtggaggactacttcagcctgatgaggaagggcgacttcggctccaacgatctgatcgaaaatatcaagaccaagctgagcgccgcagagaagctgctgggcacaaagtaccaggagacagccaaggacatcttcaagaaggatgagaactccaagctgatcaaggagctgctggacgccaccaagcagttccagcactttatcaagccactgctgggcacaggcgaggaggcagatcgggacctggtgttctacggcgattttctgcccctgtatgagaagtttgaggagctgaccctgctgtataacaaggtgcggaatagactgacacagaagccctattccaaggacaagatccgcctgtgcttcaacaagcctaagctgatgacaggctgggtggattccaagaccgagaagtctgacaacggcacacagtacggcggctatctgtttcggaagaagaatgagatcggcgagtacgattattttctgggcatctctagcaaggcccagctgttcagaaagaacgaggccgtgatcggcgactacgagaggctggattactatcagccaaaggccaataccatctacggctctgcctatgagggcgagaacagctacaaggaggacaagaagcggctgaacaaagtgatcatcgcctatatcgagcagatcaagcagacaaacatcaagaagtctatcatcgagtccatctctaagtatcctaatatcagcgacgatgacaaggtgaccccatcctctctgctggagaagatcaagaaggtgtctatcgacagctacaacggcatcctgtccttcaagtcttttcagagcgtgaacaaggaagtgatcgataacctgctgaaaaccatcagccccctgaagaacaaggccgagtttctggacctgatcaataaggattatcagatcttcaccgaggtgcaggccgtgatcgacgagatctgcaagcagaaaaccttcatctactttccaatctccaacgtggagctggagaaggagatgggcgataaggacaagcccctgtgcctgttccagatcagcaataaggatctgtccttcgccaagacctttagcgccaacctgcggaagaagagaggcgccgagaatctgcacacaatgctgtttaaggccctgatggagggcaaccaggataatctggacctgggctctggcgccatcttctacagagccaagagcctggacggcaacaagcccacacaccctgccaatgaggccatcaagtgtaggaacgtggccaataaggataaggtgtccctgttcacctacgacatctataagaacaggcgctacatggagaataagttcctgtttcacctgagcatcgtgcagaactataaggccgccaatgactccgcccagctgaacagctccgccaccgagtatatcagaaaggccgatgacctgcacatcatcggcatcgataggggcgagcgcaatctgctgtactattccgtgatcgatatgaagggcaacatcgtggagcaggactctctgaatatcatcaggaacaatgacctggagacagattaccacgacctgctggataagagggagaaggagcgcaaggccaaccggcagaattgggaggccgtggagggcatcaaggacctgaagaagggctacctgagccaggccgtgcaccagatcgcccagctgatgctgaagtataacgccatcatcgccctggaggatctgggccagatgtttgtgacccgcggccagaagatcgagaaggccgtgtaccagcagttcgagaagagcctggtggataagctgtcctacctggtggacaagaagcggccttataatgagctgggcggcatcctgaaggcctaccagctggcctctagcatcaccaagaacaattctgacaagcagaacggcttcctgttttatgtgccagcctggaatacaagcaagatcgatcccgtgaccggctttacagacctgctgcggcccaaggccatgaccatcaaggaggcccaggacttctttggcgccttcgataacatctcttacaatgacaagggctatttcgagtttgagacaaactacgacaagtttaagatcagaatgaagagcgcccagaccaggtggacaatctgcaccttcggcaatcggatcaagagaaagaaggataagaactactggaattatgaggaggtggagctgaccgaggagttcaagaagctgtttaaggacagcaacatcgattacgagaactgtaatctgaaggaggagatccagaacaaggacaatcgcaagttctttgatgacctgatcaagctgctgcagctgacactgcagatgcggaactccgatgacaagggcaatgattatatcatctctcctgtggccaacgccgagggccagttctttgactcccgcaatggcgataagaagctgccactggatgcagacgcaaacggagcctacaatatcgcccgcaagggcctgtggaacatccggcagatcaagcagaccaagaacgacaagaagctgaatctgagcatctcctctacagagtggctggatttcgtgcgggagaagccttacctgaag

SEQ ID NO: 228 - protein, 1368 aa, Streptococcus pyogenes [sequence omitted]

SEQ ID NO: 229 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 230 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 231 - protein, 1233 aa, Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 232 - protein, 1246 aa, Lachnospiraceae bacterium
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 233 - protein, 1228 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 234 - protein, 1300 aa, Francisella tularensis; identical to SEQ ID NO: 229

SEQ ID NO: 235 - protein, 1477 aa, Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 236 - protein, 1352 aa, Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 237 - protein, 1206 aa, Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 238 - protein, 1238 aa, Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 239 - protein, 1282 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 240 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 241 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 242 - DNA, 39 bp, Homo sapiens
  source 1..39: mol_type = genomic DNA
tatctgtttggagctgttggcatcatgttcctggggctc

SEQ ID NO: 243 - DNA, 39 bp, Homo sapiens
  source 1..39: mol_type = genomic DNA
gagccccaggaacatgatgccaacagctccaaacagata

SEQ ID NO: 244 - RNA, 43 nt, synthetic construct; identical to SEQ ID NO: 209
  misc_feature 1..43: note = crRNA sequence for genomic safe harbor locus shown in FIG. 1. The crRNA can be modified at the 3' end with an 18 atom oligoethylene glycol spacer and a 3' terminal thiol.
  source 1..43: mol_type = other RNA

SEQ ID NO: 245 - DNA, 92 bp, Homo sapiens
  source 1..92: mol_type = genomic DNA
gagggattccctacctcccgtgctatctgtttggagctgttggcatcatgttcctggggctccgaagctgctgggaaacaggagtgttctgc

SEQ ID NO: 246 - DNA, 92 bp, Homo sapiens
  source 1..92: mol_type = genomic DNA
gcagaacactcctgtttcccagcagcttcggagccccaggaacatgatgccaacagctccaaacagatagcacgggaggtagggaatccctc

SEQ ID NO: 247 - DNA, 100 bp, synthetic construct
  misc_feature 1..100: note = Synthetic homology template for target sequence of GSH locus shown in FIG. 1 with PAM mutation
  source 1..100: mol_type = other DNA
gagggattccctacctcccgtgctatctgttcggagctgttggcatcatggcggccgcttcctggggctccgaagctgctgggaaacaggagtgttctgc

SEQ ID NO: 248 - DNA, 100 bp, synthetic construct
  misc_feature 1..100: note = Synthetic homology template for non-target sequence of GSH locus shown in FIG. 1 with PAM mutation. The synthetic homology template can be modified at the 3' end with an 18 atom oligoethylene glycol spacer and a 3' terminal thiol.
  source 1..100: mol_type = other DNA
gcagaacactcctgtttcccagcagcttcggagccccaggaagcggccgccatgatgccaacagctccgaacagatagcacgggaggtagggaatccctc

SEQ ID NO: 249 - DNA, 27 bp, Homo sapiens
  source 1..27: mol_type = genomic DNA
tttggagctgttggcatcatgttcctg

SEQ ID NO: 250 - DNA, 27 bp, Homo sapiens
  source 1..27: mol_type = genomic DNA
tttggagctgttggcatcttgttcctg

SEQ ID NO: 251 - DNA, 37 bp, Homo sapiens
  source 1..37: mol_type = genomic DNA
ttgttgtttatccaaacctcctaaatgatacgtgttt

SEQ ID NO: 252 - DNA, 37 bp, Homo sapiens
  source 1..37: mol_type = genomic DNA
aaacacgtatcatttaggaggtttggataaacaacaa

SEQ ID NO: 253 - RNA, 41 nt, synthetic construct; identical to SEQ ID NO: 211
  misc_feature 1..41: note = crRNA sequence for GSH locus shown in FIG. 3. The crRNA can be modified at the 3' end with an 18 atom oligoethylene glycol spacer and a 3' terminal thiol.
  source 1..41: mol_type = other RNA

SEQ ID NO: 254 - DNA, 92 bp, Homo sapiens
  source 1..92: mol_type = genomic DNA
gcttagtgcttatttattttaggttgttgtttatccaaacctcctaaatgatacgtgtttggaacaagtaacagcattgttccttgatgttg

SEQ ID NO: 255 - DNA, 92 bp, Homo sapiens
  source 1..92: mol_type = genomic DNA
caacatcaaggaacaatgctgttacttgttccaaacacgtatcatttaggaggtttggataaacaacaacctaaaataaataagcactaagc

SEQ ID NO: 256 - DNA, 100 bp, synthetic construct
  misc_feature 1..100: note = Synthetic homology template for target sequence of GSH locus shown in FIG. 3 with PAM mutation
  source 1..100: mol_type = other DNA
gcttagtgcttatttattttaggttgttgttcatccaaacctcctaaatggcggccgcatacgtgtttggaacaagtaacagcattgttccttgatgttg

SEQ ID NO: 257 - DNA, 100 bp, synthetic construct
  misc_feature 1..100: note = Synthetic homology template for non-target sequence of GSH locus shown in FIG. 3 with PAM mutation. The synthetic homology template can be modified at the 3' end with an 18 atom oligoethylene glycol spacer and a 3' terminal thiol.
  source 1..100: mol_type = other DNA
caacatcaaggaacaatgctgttacttgttccaaacacgtatgcggccgccatttaggaggtttggatgaacaacaacctaaaataaataagcactaagc

SEQ ID NO: 258 - DNA, 53 bp, Homo sapiens
  source 1..53: mol_type = genomic DNA
gcgagcaagctcagtttacacccgatccactggggagcaggaaatatctgtgg

SEQ ID NO: 259 - DNA, 53 bp, Homo sapiens
  source 1..53: mol_type = genomic DNA
ccacagatatttcctgctccccagtggatcgggtgtaaactgagcttgctcgc

SEQ ID NO: 260 - RNA, 41 nt, synthetic construct
  misc_feature 1..41: note = crRNA sequence for GSH locus shown in FIG. 4. The crRNA can be modified at the 3' end with an 18 atom oligoethylene glycol spacer and a 3' terminal thiol.
  source 1..41: mol_type = other RNA
taatttctactcttgtagatcacccgatccactggggagca

SEQ ID NO: 261 - DNA, 88 bp, synthetic construct
  misc_feature 1..88: note = Synthetic homology template for non-target sequence of GSH locus shown in FIG. 4
  source 1..88: mol_type = other DNA
ccacttgagtccgtgtcacaagcccacagatatttcctgcgcggccgctccccagtggatcgggtgtaaactgagcttgctcgctcgg

SEQ ID NO: 262 - DNA, 88 bp, synthetic construct
  misc_feature 1..88: note = Synthetic homology template for target sequence of GSH locus shown in FIG. 4
  source 1..88: mol_type = other DNA
cgagcaagctcagttcacacccgatccactggggagcagggcggccgcaaatatctgtgggcttgtgacacggactcaagtgggctgg

SEQ ID NO: 263 - DNA, 43 bp, Homo sapiens
  source 1..43: mol_type = genomic DNA
aagctcagtttacacccgatccactggggagcaggaaatatct

SEQ ID NO: 264 - RNA, 36 nt, synthetic construct
  misc_feature 1..36: note = crRNA to use with Cas9 described in Example 1. The crRNA can be modified at the 5' end with an 18 atom oligoethylene glycol spacer and a 5' terminal thiol.
  source 1..36: mol_type = other RNA
cacccgatccactggggagcgttttagagctatgct